(19) United States
(12) Patent Application Publication
(10) Pub. No.: US 2003/0129654 A1
(43) Pub. Date: Jul. 10, 2003

(54) Coded particles for multiplexed analysis of biological samples

(76) Inventors: Ilya Ravkin, Palo Alto, CA (US); Simon Goldbard, San Jose, CA (US); Michael A. Zarowitz, San Carlos, CA (US); William C. Hyun, San Francisco, CA (US)

Correspondence Address: KOLISCH HARTWELL, P.C., 520 S.W. YAMHILL STREET, SUITE 200, PORTLAND, OR, 97204, US

(21) Appl. No.: 10/273,605

(22) Filed: Oct. 18, 2002

Related U.S. Application Data
(63) Continuation-in-part of application No. 09/549,970, filed on Apr. 14, 2000.
(63) Continuation-in-part of application No. 09/694,077, filed on Oct. 19, 2000.
(63) Continuation-in-part of application No. 10/120,900, filed on Apr. 10, 2002.
(60) Provisional application No. 60/343,682, filed on Oct. 26, 2001.
(60) Provisional application No. 60/343,685, filed on Oct. 26, 2001.
(60) Provisional application No. 60/344,482, filed on Oct. 26, 2001.
(60) Provisional application No. 60/413,675, filed on Sep. 24, 2002.
(60) Provisional application No. 60/359,207, filed on Feb. 21, 2002.
(60) Provisional application No. 60/345,606, filed on Oct. 26, 2001.
(60) Provisional application No. 60/344,483, filed on Oct. 26, 2001.
(60) Provisional application No. 60/170,947, filed on Dec. 15, 1999.
(60) Provisional application No. 60/129,664, filed on Apr. 15, 1999.
(60) Provisional application No. 60/348,025, filed on Oct. 26, 2001.
(60) Provisional application No. 60/348,027, filed on Oct. 26, 2001.
(60) Provisional application No. 60/362,001, filed on Mar. 5, 2002.
(60) Provisional application No. 60/362,055, filed on Mar. 5, 2002.
(60) Provisional application No. 60/362,238, filed on Mar. 5, 2002.
(60) Provisional application No. 60/370,313, filed on Apr. 4, 2002.
(60) Provisional application No. 60/383,091, filed on May 23, 2002.
(60) Provisional application No. 60/383,092, filed on May 23, 2002.
(60) Provisional application No. 60/413,407, filed on Sep. 24, 2002.

(30) Foreign Application Priority Data
Oct. 18, 2001 (WO) PCT/US01/51413

(51) Int. Cl. G01N 33/53; C12M 1/34; G01N 33/543

(52) U.S. Cl. 435/7.1; 435/287.1; 436/518

(57) ABSTRACT

Systems including apparatus, methods, compositions, and kits for multiplexed analysis of biological samples or reagents using coded particles. The coded particles may be used to form positionally flexible arrays of samples and/or reagents in which the samples and/or reagents are identified by codes on the particles.

### CROSS-REFERENCES TO PRIORITY APPLICATIONS

This application is a continuation-in-part of the following U.S. patent applications: Ser. No. 09/549,970, filed Apr. 14, 2000; Ser. No. 09/694,077, filed Oct. 19, 2000; and Ser. No. 10/120,900, filed Apr. 10, 2002. This application also claims the benefit under 35 U.S.C. § 119(e) of the following U.S. provisional patent applications: Serial No. 60/343,682, filed Oct. 26, 2001; Serial No. 60/343,685, filed Oct. 26, 2001; Serial No. 60/344,482, filed Oct. 26, 2001; Serial No. 60/344,483, filed Oct. 26, 2001; Serial No. 60/345,606, filed Oct. 26, 2001; Serial No. 60/359,207, filed Feb. 21, 2002; and Serial No. 60/413,675, filed Sep. 24, 2002.

U.S. patent application Ser. No. 09/549,970, in turn, claims the benefit under 35 U.S.C. § 119(e) of the following U.S. provisional patent applications: Serial No. 60/129,664, filed Apr. 15, 1999; and Serial No. 60/170,947, filed Dec. 15, 1999.

U.S. patent application Ser. No. 09/694,077, in turn, is a continuation-in-part of U.S. patent application Ser. No. 09/549,070, which claims priority as described above.

U.S. patent application Ser. No. 10/120,900, in turn, claims the benefit under 35 U.S.C. § 120 of PCT Application Serial No. PCT/US01/51413, filed Oct. 18, 2001, and published as Publication No. WO 02/37944 on May 16, 2002, which in turn claims priority from the following U.S. provisional patent applications: Serial No. 60/241,714, filed Oct. 18, 2000; Serial No. 60/259,416, filed Dec. 28, 2000; Serial No. 60/293,863, filed May 24, 2001; Serial No. 60/299,267, filed Jun. 18, 2001; Serial No. 60/299,810, filed Jun. 20, 2001; Serial No. 60/307,649, filed Jul. 24, 2001; Serial No. 60/307,650, filed Jul. 24, 2001; Serial No. 60/310,540, filed Aug. 6, 2001; Serial No. 60/317,409, filed Sep. 4, 2001; Serial No. 60/318,156, filed Sep. 7, 2001; and Serial No. 60/328,614, filed Oct. 10, 2001.

The above-identified U.S., PCT, and provisional priority patent applications are all incorporated herein by reference in their entirety for all purposes.

### CROSS-REFERENCES TO RELATED APPLICATIONS

This application incorporates by reference in its entirety for all purposes the following U.S. patent application: Ser. No. 10/238,914, filed Sep. 9, 2002.

This application also incorporates by reference in their entirety for all purposes the following U.S. provisional patent applications: Serial No. 60/348,025, filed Oct. 26, 2001; Serial No. 60/348,027, filed Oct. 26, 2001; Serial No. 60/362,001, filed Mar. 5, 2002; Serial No. 60/362,055, filed Mar. 5, 2002; Serial No. 60/362,238, filed Mar. 5, 2002; Serial No. 60/370,313, filed Apr. 4, 2002; Serial No. 60/383,091, filed May 23, 2002; Serial No. 60/383,092, filed May 23, 2002; and Serial No. 60/413,407, filed Sep. 24, 2002.

This application also incorporates by reference in its entirety for all purposes the following PCT patent application: Serial No. PCT/US00/10181, filed Apr. 14, 2000, and published as Publication No. WO 00/63419 on Oct. 26, 2000.

### Additional Materials

This application also incorporates by reference in their entirety for all purposes the following U.S. Pat. Nos. 3,772,099, issued Nov. 13, 1973; No. 3,897,284, issued Jul. 29, 1975; No. 3,964,294, issued Jun. 22, 1976; No. 3,966,599, issued Jun. 29, 1976; No. 3,980,561, issued Sep. 14, 1976; No. 4,053,433, issued Oct. 11, 1977; No. 4,087,327, issued May 2, 1978; No. 4,131,064, issued Dec. 26, 1978; No. 4,197,104, issued Apr. 8, 1980; No. 4,329,393, issued May 11, 1982; No. 4,343,904, issued Aug. 10, 1982; No. 4,363,965, issued Dec. 14, 1982; No. 4,390,452, issued Jun. 28, 1983; No. 4,469,623, issued Sep. 4, 1984; No. 4,634,675, issued Jan. 6, 1987; No. 4,640,035, issued Feb. 3, 1987; No. 4,649,114, issued Mar. 10, 1987; No. 4,652,395, issued Mar. 24, 1987; No. 4,727,040, issued Feb. 23, 1988; No. 4,833,083, issued May 23, 1989; No. 4,888,294, issued Dec. 19, 1989; No. 4,906,577, issued Mar. 6, 1990; No. 4,921,792, issued May 1, 1990; No. 4,963,490, issued Oct. 16, 1990; No. 4,982,739, issued Jan. 8, 1991; No. 5,019,512, issued May 28, 1991; No. 5,079,161, issued Jan. 7, 1992; No. 5,081,036, issued Jan. 14, 1992; No. 5,096,814, issued Mar. 17, 1992; No. 5,100,783, issued Mar. 31, 1992; No. 5,100,799, issued Mar. 31, 1992; No. 5,114,853, issued May 19, 1992; No. 5,126,269, issued Jun. 30, 1992; No. 5,233,369, issued Aug. 3, 1993; No. 5,409,839, issued Apr. 25, 1995; No. 5,451,505, issued Sep. 19, 1995; No. 5,486,855, issued Jan. 23, 1996; No. 5,571,410, issued Nov. 5, 1996; No. 5,708,153, issued Jan. 13, 1998; No. 5,741,462, issued Apr. 21, 1998; No. 5,760, 394, issued Jun. 2, 1998; No. 5,770,455, filed Jun. 23, 1998; No. 5,780,258, issued Jul. 14, 1998; issued Jun. 23, 1998; No. 5,817,751, issued Oct. 6, 1998; No. 5,840,485, issued Nov. 24, 1998; No. 5,961,923, issued Oct. 5, 1999; No. 5,981,180, issued Nov. 9, 1999; No. 5,989,835, issued Nov. 23, 1999; No. 5,990,479, issued Nov. 23, 1999; No. 6,025,200, issued Feb. 15, 2000; No. 6,100,026, issued Aug. 8, 2000; and No. 6,103,479, issued Aug. 15, 2000.

This application also incorporates by reference in their entirety for all purposes the following PCT Patent Applications: Serial No. PCT/IL97/00105, filed Mar. 20, 1997; Serial No. PCT/US98/21562, filed Oct. 14, 1998; Serial No. PCT/US98/22785, filed Oct. 27, 1998; Serial No. PCT/US99/00918, filed Jan. 15, 1999; Serial No. PCT/US99/01315, filed Jan. 22, 1999; Serial No. PCT/GB99/00457, filed Feb. 15, 1999; Serial No. PCT/US99/14387, filed Jun. 24, 1999; Serial No. PCT/GB99/02108, filed Jul. 2, 1999; Serial No. PCT/SE99/01836, filed Oct. 12, 1999; Serial No. PCT/US99/31022, filed Dec. 28, 1999; Serial No. PCT/US00/25457, filed Sep. 18, 2000; Serial No. PCT/US00/27121, filed Oct. 2, 2000; and Serial No. PCT/US00/41049, filed Oct. 2, 2000.

### FIELD OF THE INVENTION

The invention relates to systems using coded particles. More particularly, the invention relates to systems using coded particles for multiplexed analysis of biological samples or reagents.

### BACKGROUND

Analysis of biological systems often relies on repetitive testing performed in series and/or in parallel. For example, in drug screens a primary target is repetitively tested for interaction with a library of candidate drugs. When interacting candidates are identified, such candidates may be further tested against a set of potential secondary targets to determine specificity of the candidates for the primary target. Similarly, in clinical diagnostics, a patient sample, or more typically a set of samples from different patients, may be repetitively analyzed to measure different aspects of each sample. Furthermore, genome analysis, synthesis of high complexity libraries of chemicals, and engineering of new cell lines, among others, have provided additional impetus for repetitive testing.

The large number of tests that need to be performed for discovery and clinical diagnosis has spurred development of more efficient testing methods. Substantial effort has been directed toward the speed with which tests are performed and results measured. For example, microtiter plates (or microplates) of very high density have been developed to positionally identify and compartmentalize a large number of samples in an array format. Instrumentation to process and analyze samples at high speed in this format has also been developed. However, with each test occupying a separate well of a microplate, a prohibitively large number of microplates and manipulations may be required to meet goals of discovery. For example, the analysis of 100,000 samples a day in separate sample wells, common in high-throughput screening, requires a stack of standard-sized 96-well microplates over 40 feet high each day and over 3 miles high each year, as well as the associated reagents.

Position-dependent microarrays, such as “gene chips,” provide another array format in which repetitive testing may be conducted. Similar to microplates, samples and/or reagents are disposed at distinct, defined positions within a fixed array. However, in contrast to microplates, fluid barriers between the reagents or samples are not included. Instead, the samples or reagents are immobilized at defined positions through attachment to the microarray substrate. Accordingly, the samples or reagents may be positioned at very high density on the microarray substrate and processed in parallel in a shared fluid volume for multiplexed analysis.

Despite their conceptual attractiveness, position-dependent microarrays may not be suitable for many types of testing. For example, the equipment to form and analyze such microarrays is expensive. Accordingly, microarrays are often produced as a standardized array of test reagents. This array cannot be modified readily to meet the needs of an individual user. In addition, microarrays are not suitable for tests of some types of samples or reagents. For example, different type of cells may prefer different substrates for attachment and/or growth. As a result, a microarray substrate may be limited in the types of cells that can be analyzed on the substrate. Therefore, new array formats are needed for multiplexed analysis of samples and/or reagents.

### SUMMARY OF THE INVENTION

Systems including apparatus, methods, compositions, and kits are provided for multiplexed analysis of biological samples or reagents using coded particles. The coded particles may be used to form positionally flexible arrays of samples and/or reagents in which the samples and/or reagents are identified by codes on the particles.

### BRIEF DESCRIPTION OF THE DRAWINGS

FIG. 1 is a perspective view of a coded particle associated with a sample, in accordance with aspects of the invention.

FIG. 2 is a schematic View of a system for multiplexed cell analysis using coded particles, in accordance with aspects of the invention.

FIG. 3 is a perspective view of an alternative embodiment of the coded particle of FIG. 1 in which the noncoding portion includes a recess that forms an interior compartment, in accordance with aspects of the invention.

FIG. 4 is a perspective view of another embodiment of the coded particle of FIG. 1 in which the particle includes plural interior compartments and a magnetic portion, in accordance with aspects of the invention.

FIG. 5 is a perspective view of yet another embodiment of the coded particle of FIG. 1 in which the noncoding portion includes ridges and grooves, in accordance with aspects of the invention.

FIG. 6 is a side elevation view of the coded particle of FIG. 5.

FIG. 7 is a side elevation view of an alternative embodiment of the coded particle of FIG. 5, in accordance with aspects of the invention.

FIG. 8 is a side elevation view of another embodiment of the coded particle of FIG. 5, in accordance with aspects of the invention.

FIG. 9 is a side elevation view of an embodiment of a coded-particle intermediate having a plurality of differentially sensitive fibers attached to its noncoding portion, in accordance with aspects of the invention.

FIG. 10 is a side elevation view of a coded particle produced from the intermediate of FIG. 9 after selective removal of a subset of the fibers to form ridges and grooves, in accordance with aspects of the invention.

FIG. 11 is a side elevation view of another embodiment of the coded-particle intermediate of FIG. 9 with a different ratio of the differentially sensitive fibers attached to its noncoding portion, in accordance with aspects of the invention.

FIG. 12 is a schematic view of a system for purifying and analyzing cell components using coded particles that include a magnetic portion, in accordance with aspects of the invention.

FIG. 13 is a perspective view of a coded particle with a linear code, in accordance with aspects of the invention.

FIG. 14 is a perspective view of a planar particle having a two-dimensional code, in accordance with aspects of the invention.

FIG. 15 is a perspective view of another planar particle having a two-dimensional code, in accordance with aspects of the invention.

FIG. 16 is a perspective view of a cylindrical particle having a linear code with code elements arrayed parallel to the cylinder axis, in accordance with aspects of the invention.

FIG. 17 is a perspective view of a coded cylindrical particle with code elements arrayed in two-dimensions and perpendicular to the cylinder axis, in accordance with aspects of the invention.

FIG. 18 is a perspective view of a disc embodiment of the coded cylindrical particle of FIG. 17, in accordance with aspects of the invention.

FIG. 19 is a perspective view of another disc embodiment of a coded cylindrical particle in which code elements are defined by concentric rings in the particle, in accordance with aspects of the invention.

FIG. 20 is a partially sectional perspective view of a bead embodiment of a coded particle in which code elements are defined by concentric spherical layers, in accordance with aspects of the invention.

FIG. 21 is a perspective view of a sheet of fused fibers having a linear code defined by code elements arrayed perpendicular to the fibers, in accordance with aspects of the invention.

FIG. 22 is a perspective view of a stack of sheets aligned for cutting into individual coded particles along cutting planes, with the stack including several sheets corresponding to the sheet of FIG. 21, in accordance with aspects of the invention.

FIG. 23 is a perspective view of a coded particle formed by cutting along one of the cutting planes indicated in FIG. 22.

FIG. 24 is a perspective view of cylindrical coded particles being cut from a coded sheet and being combined with cylindrical particles having distinct codes for detection in a capillary tube, in accordance with aspects of the invention.

FIG. 25 is a top plan view of a coded particle having a binary code with 16 bits of information, in accordance with aspects of the invention.

FIGS. 26A-F are fragmentary sectional views of exemplary fabrication intermediates (A-E) and the coded particle (F) of FIG. 25 viewed generally along line 26-26 of FIG. 25, in accordance with aspects of the invention.

FIGS. 27A-E are sectional views of exemplary intermediates (A-D) and a finished coded particle (E) formed by a method using soft lithography, in accordance with aspects of the invention.

FIG. 28 is a schematic view of a method for making coded particles from film using a film/sample sandwich, in accordance with aspects of the invention.

FIG. 29 is a sectional side view of the film/sample sandwich of FIG. 28, taken generally along line 29-29 in FIG. 28.

FIG. 30 is a schematic view of a method for producing plural particles having the same code, in accordance with aspects of the invention.

FIG. 31 is a schematic view of a method for producing plural particles having different codes, in accordance with aspects of the invention.

FIG. 32 is a top plan view of a particle having a color code, in accordance with aspects of the invention.

FIG. 33 is a schematic view illustrating a diffraction pattern formed at defined angles by monochromatic light transmitted through an interference filter, in accordance with aspects of the invention.

FIG. 34 is a schematic isometric view of a coded particle that includes a code formed by plural linear diffraction gratings, in accordance with aspects of the invention.

FIG. 35 is a sectional side elevation view of the coded particle of FIG. 34, taken generally along line 35-35 in FIG. 34.

FIG. 36 is a schematic isometric view of another coded particle that includes a code formed by plural linear diffraction gratings, in accordance with aspects of the invention.

FIG. 37 is a schematic top plan view of a coded particle that includes a code formed by circular diffraction gratings, in accordance with aspects of the invention.

FIG. 38 is a schematic isometric view of a cylindrical coded particle with a code formed by linear diffraction gratings, in accordance with aspects of the invention.

FIG. 39 is a schematic isometric view of a mold that may be used to form the coded particle of FIG. 34, in accordance with aspects of the invention.

FIG. 40 is a sectional side elevation view of the mold of FIG. 39, taken generally along line 40-40 in FIG. 39.

FIG. 41 is a schematic top plan view of a molding matrix, in accordance with aspects of the invention.

FIG. 42 is a schematic, fragmentary sectional view of an embodiment of the molding matrix of FIG. 41, taken generally along line 42-42 in FIG. 41, in accordance with aspects of the invention.

FIG. 43 is a schematic, fragmentary sectional view of an alternative embodiment of the molding matrix of FIG. 41, taken generally along line 43-43 in FIG. 41, in accordance with aspects of the invention.

FIG. 44 is a schematic top plan view of an embodiment of a grid-field mold module with a first grid spacing, in accordance with aspects of the invention.

FIG. 45 is a side elevation view of the grid-field mold module of FIG. 44.

FIG. 46 is a schematic top plan view of the grid-field mold module of FIG. 44 abutting, and aligned with, a second grid-field mold module that includes a second grid spacing, in accordance with aspects of the invention.

FIG. 47 is a schematic, fragmentary top plan view of a molding matrix formed with grid-field mold modules, in accordance with aspects of the invention.

FIG. 48 is a schematic side elevation view of an apparatus for producing cylindrical coded particles that include diffraction gratings, in accordance with aspects of the invention.

FIG. 49 is a schematic top plan view of a molding wheel used in the apparatus of FIG. 48, viewed generally along line 49-49 in FIG. 48.

FIG. 50 is a fragmentary sectional view of the junction between the molding wheels used in the apparatus of FIG. 48 as the molding wheels form a particle, viewed generally along line 50-50 in FIG. 48.

FIG. 51 is a schematic view of monochromatic light paths diffracted through an interference code on a particle, in accordance with aspects of the invention.

FIG. 52 is a top plan view of a die used for forming plural particles with imprinted codes, in accordance with aspects of the invention.

FIG. 53 is a fragmentary perspective view of the die of FIG. 52, showing a region of the die (“53” in FIG. 52) used to form a particle having a group of pyramidal features, in accordance with aspects of the invention.

FIG. 54 is a fragmentary perspective view of another die for forming particles with imprinted codes, showing a region of the die used to form a particle code having a group of conical features, in accordance with aspects of the invention.

FIG. 55 is a schematic side view of a system for producing imprinted particles, in accordance with aspects of the invention.

FIG. 56 is a schematic side view of a system for reading topographic codes using illumination originating from a side facing away from a surface relief feature, in accordance with aspects of the invention.

FIG. 57 is a schematic side view of a system for reading topographic codes using illumination originating from a side facing toward a surface relief feature, in accordance with aspects of the invention.

FIG. 58 is an isometric, fragmentary sectional view of a die with a pyramidal die feature formed anisotropically in monocrystalline silicon that has a (100) crystal orientation and a longitudinal ridge direction of <110>, in accordance with aspects of the invention.

FIG. 59 is a fragmentary sectional side view of a die with an arcuate die feature formed isotropically in monocrystalline silicon, in accordance with aspects of the invention.

FIG. 60 is a schematic view of two methods for forming coded particles that include MIPs, with each code element being formed by distinct or equivalent print molecules, in accordance with aspects of the invention.

FIG. 61 is a schematic view of detection methods for measuring binding to MIPs on coded particles using either a labeled secondary antibody or a competitive binding assay, in accordance with aspects of the invention.

### DETAILED DESCRIPTION

The invention provides systems including apparatus, methods, compositions, and kits for multiplexed analysis of samples using coded particles, particularly in positionally flexible arrays. These systems may provide a variety of benefits, for example, allowing multiple samples and/or reagents to be analyzed together as a mixture. Coupled with ongoing improvements in microplates, microfluidics, and robotics, these systems may increase throughput by expanding the number of samples, sample aspects, and/or reagents that are tested or screened. Thus, the invention may be used to identify valuable therapeutic agents and to increase human understanding of biological systems, with concomitant benefits for treating human disease and improving human health. More particularly, the invention may be used with cells to identify cell types, ligands, or cell type-ligand combinations that suppress or enhance metabolic or physiological reactions of interest.

The invention may have a number of advantages over prior systems, potentially including (1) increased throughput due to multiplexing, (2) flexibility in the composition of arrays, (3) simplified handling, because there may be fewer sample containers since each container may contain many types of samples and/or reagents, (4) compatibility with existing assays and equipment, including fluid dispensers, sample handlers, and sample readers, (5) reduced consumption of expensive reagents, e.g., FISH in a tube, not on a slide, (6) increased information content due to higher density of samples and/or reagents, and (7) simultaneous testing of specificity and potency in a well.

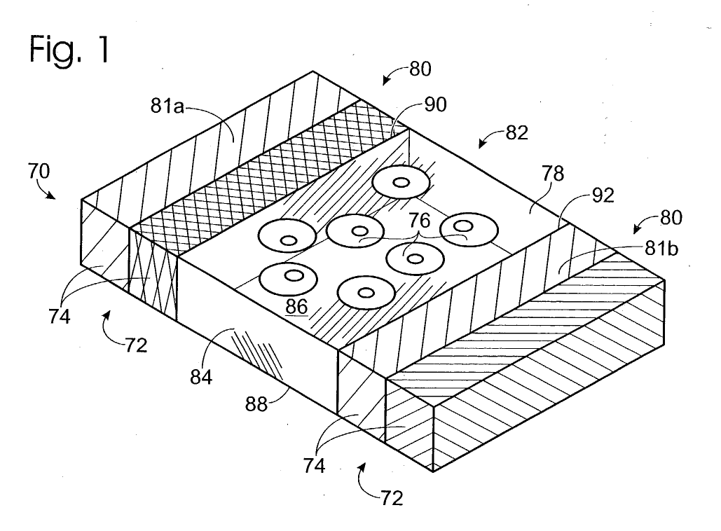

FIG. 1 shows a perspective view of an embodiment of a coded particle 70 for multiplexed analysis of biological samples. Particle 70 includes a code 72 defined by one or a plurality of code elements 74. Each code element 74 may define a portion of code 72 based on optically detectable properties of the code element. Such detectable properties may include an optical property defined by the element's interaction with light, for example, absorbance, fluorescence, reflection, scattering, and/or the like. Interaction with light also may reveal a spatial property of the code element, such as position within the particle, shape, size, and/or number, among others. Here, code 72 is a positional or spatial color code in which each code element 74 has a detectable color and a position relative to other code elements. Thus, in this embodiment the colors and relative positions of the code elements define the code. Further aspects of particle codes, including suitable codes, code elements, optical properties, and spatial properties, are described below in Section I.

Particle 70 may have any suitable size and shape. The size and shape may be selected, for example, with respect to the type of analysis performed, the complexity of the analysis, containers used to hold the particles, methods used to move the particles, methods used for reading codes and measuring experimental results, and/or so on. In preferred embodiments, particle 70 is small enough so that two or more particles can be analyzed at once in a microscopic field of view. The shape of particle 70 may be generally flat or planar, for example, the flattened or generally planar parallelepiped shown in FIG. 1. In other embodiments, particle 70 may be cylindrical, spherical, ovalloid, and/or the like. Particle 70 may be formed of any suitable material or materials, based, for example, on optical properties, biocompatibility, suitability for manufacturing, and/or so on. In some embodiments, particle 70 may be formed at least partially or at least substantially of glass or plastic. Further aspects of particle sizes, shapes, and materials are included below in Sections II-IV and X.

Particle 70 is associated with a sample(s) and/or reagent(s) to link code 72 to the sample/reagent. Accordingly, code 72 identifies the associated sample/reagent, thereby allowing the sample/reagent to be tracked during processing and analysis. Due to the identifying code, the particle-associated sample/reagent may be mixed with other samples/reagents that are associated with other particles having distinct codes, for multiplexed processing and analysis of the samples/reagents in a mixture. Here, particle 70 is associated with a sample, cells 76, through attachment of cells 76 to surface 78 of the particle. Further aspects of samples and reagents and their association with particles are included below in Section VIII.

The particle surface may have any suitable properties. For example, surface 78 may at least partially form the code, may facilitate sample/reagent association and/or retention, and/or may channel fluid to particle-associated samples/reagents, among others. In addition, the sample/reagent may be associated with, and/or analyzed on, only a portion of surface 78, as shown in FIG. 1, all of surface 78, and/or below the surface and thus internal to the particle.

Particle 70 may be formed by one or more structural components. For example, particle 70 may include a plurality of joined structural components. The structural components may form a coding portion 80 having adjacent or spaced code elements 74 to define one coding region, or, as shown here, plural spaced coding regions 81a,b. The structural components also may form a discrete noncoding portion 82, for example using noncoding element 84. The coding portion and noncoding portion may be formed of similar or different materials and may have similar or distinct physical, chemical, optical, and/or surface properties, among others. For example, in particle 70, cells may attach preferentially to noncoding element 84. Alternatively, or in addition, noncoding element 84 may be colorless so that it doesn't interfere with reading a color-based code and/or measurement of a cell characteristic. Further aspects of particle manufacture and particle structure are included below in Sections VII and X, including particles formed at least partially from a composite of fused and stretched fibers, film, or molecular imprinted materials, and particles produced by stamping, molding, etching, soft lithography, and/or photolithography, among others.

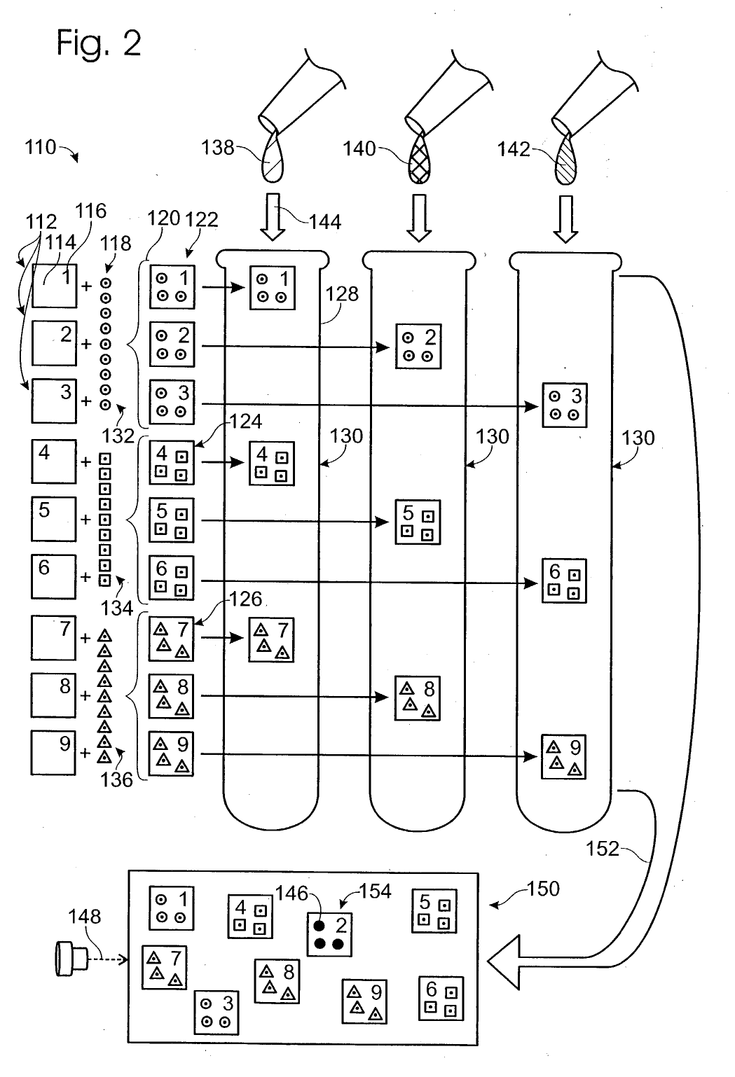

FIG. 2 shows a system 110 for multiplexed analysis using coded particles 112, in accordance with aspects of the invention. Coded particle 112 is a particle 114 that includes a detectable code 116. The particle may provide a support structure with which a sample 118, in this case a cellular sample, and/or test reagents, such as cell-analysis materials, may be associated, shown at 120, to form a particle assembly 122. The association maintains a linkage between the code and the sample/test reagent during some or all of the analysis. Thus, the code may identify the sample, the reagent, and/or other aspects of the analysis, such as assay steps or conditions. Further aspects of associating samples and/or reagents with coded particles are described below in Section VIII.

Particle assemblies with distinct codes, such as assemblies 122, 124, and 126, may be combined at an assay site, generally in a container 128, to form a coded array 130. The coded array may be positionally flexible, also termed nonpositional, meaning that the particle assemblies within the array may have an arbitrary or random distribution relative to one another. A nonpositional array may allow more than one distinct sample, e.g., cell populations 132, 134, 136, to be treated, analyzed, and/or screened together. Thus, a library of samples and/or test reagents may be formed as a nonpositional array.

Samples may be analyzed by contacting the samples (in this case, the cells) with test reagents, such as modulators 138, 140, 142. Contacting, shown at 144, may test interaction between the sample and test reagents, for example, binding of a ligand to a receptor. With cellular samples, the test reagents may be cell-analysis materials, such as (1) modulators, (2) ligands/receptors, (3) transfection materials, (4) cell selectors, (5) local capturing agents, (6) biological entities (such as cells, viruses, tissues, etc., and components thereof) and/or (7) labels. Modulators (1) and/or ligands/receptors (2) may alter the cells themselves, may physically interact with the cells, and/or may modulate or define interaction of the cells with other cell-analysis materials. Transfection materials (3) may introduce a foreign test material into the cells to affect and/or report one or more properties of the cells. Cell selectors (4) may purify, limit analysis to, and/or identify certain cells in a larger cell population. Local capturing agents (5) may allow analysis of components attached to, and/or released from, cells. Biological entities (6) also may function as cell-analysis material, for example, to carry and/or express members of a library of cell-analysis materials and/or to allow analysis of cell-cell interactions. Labels (7) may facilitate detection of cells, cell structures, cell components, and/or cell-analysis materials. Further aspects of cell-analysis materials are included in the patent applications identified above under Cross-References and incorporated herein by reference, particularly U.S. patent application Ser. No. 10/120,900, filed Apr. 10, 2002.

The analysis with coded particles may be determined by the choice of samples, reagents, and the timing and duration of exposure of the reagents to the samples and/or particles. Reagents may contact the sample before, during, and/or after associating the sample with the particles. For example, exposure of reagents to particles before association of the particles with samples may link the reagents to the particles in a sample-independent manner, termed pre-association. Thus, the code on each particle may relate information about reagents linked to the particles, and a coded, nonpositional array of test reagents may be formed prior to sample association. The array may be considered a coded library of reagents.

Exposing samples to reagents may produce or alter a detectable sample characteristic 146, such as interaction (for example, binding) with the test reagent. With a cellular sample, the characteristic may be the presence, absence, level, distribution, appearance, behavior, and/or other property of cell components, cell structures, or cells, among others.

Reading codes and measuring cell characteristics for the particle assemblies, shown at 148, are performed as part of the analysis. These reading and measuring steps may be performed on each individual nonpositional coded array 130, or with appropriate code complexity, as shown here, on a nonpositional mixture 150 produced by combining nonpositional coded arrays, shown at 152. Reading a code and measuring a cell characteristic for a particle assembly allows information related by the code to be linked to the cell characteristic. For example, as shown in FIG. 1, each code identifies the cell population (and cell type) 132, 134, or 136 associated with a particular particle. Furthermore, each code identifies a modulator, 138, 140, or 142, to which the cells were exposed in each coded array. Thus, in this example, the altered cell characteristic 146 and code (“2”) shared by particle assembly 154 link cell type 132 and modulator 140 to the altered characteristic. Therefore, this exemplary analysis indicates that modulator 140 specifically modulates cell population 132 relative to cell populations 134 and 136. Exemplary methods for performing sample assays, reading codes, and measuring sample characteristics, particularly cell characteristics, are described in more detail below in Section IX and in the patent applications identified above under Cross-References and incorporated herein by reference, particularly U.S. patent application Ser. No. 09/694,077, filed Oct. 19, 2000; and U.S. patent application Ser. No. 10/120,900, filed Apr. 10, 2002.

The following sections describe further aspects of the invention: (I) codes, (II) particle size, (III) particle shape, (IV) particle materials, (V) particle surfaces, (VI) particle manipulation, (VII) particle manufacture, (VIII) associating samples/reagents with particles, (IX) sample analysis using coded particles, and (X) examples.

I. Codes

The particles each may include at least one detectable code. The code generally comprises any mechanism capable of distinguishing different particles. The code may be based on the size, shape, composition, appearance, and/or behavior of the particle, or portions thereof. The code may be an optically detectable code or an optically detectable positional color code, among others. The code may appear (i.e., be repeated) at more than one position on the particle, and two or more different codes, usable for two or more different purposes, may appear on the same particle. Alternatively, or in addition, the code may be at least partially determined by other physical, chemical, electrical, and/or magnetic properties.

The code may be nonpositional. A nonpositional code relates to overall features and/or subfeatures of a particle that are not defined by position within the particle. These features and subfeatures may include an optical property, particle size, shape, composition, and/or other detectable property. Exemplary nonpositional codes may include using at least two different materials, where the materials differ in absorption, fluorescence, intrinsic polarization, diffraction, reflectivity, and/or any other measurably distinct property or characteristic (or indicium). These nonpositional codes may be read by determining the presence and/or other properties of signals from the different materials, for example, by measuring intensity as a function of wavelength for the particles.

Alternatively, or in addition, the code may be positional (also termed spatial). A positional code is based on the presence, identities, amounts, shapes, sizes, and/or other properties of materials (or a single material) at different positions in the particle. These positions, which define code elements, may be random and/or predefined, and may be dependent upon the physical positioning of the code elements on the particle and/or the positions of individual code elements relative to each other. Exemplary positional codes may include positioning different amounts and/or types of materials (for example, dyes) at different positions in or on a particle, for example, at regions, spots, lines, bands, concentric circles, symbols, shapes, and the like.

Each position may provide a measurable property or indicium, such as an optical property, with the positions and optical properties together defining an optical code formed of plural code elements. For example, the optical code may include code elements with distinct (or distinguishable) wavelength-dependent properties, including distinct absorptivity, transmissivity, reflectivity, refractivity, emissivity, diffractivity, and/or excitation and emission spectra, among others. A color code may include one or more colored code elements. These code elements (and the associated coding regions) are considered colored when they selectively absorb, selectively emit, selectively are excited by, and/or selectively otherwise interact with a subset of visible or near-visible (i.e., ultraviolet and/or infrared) light wavelengths. Color may be characterized and/or distinguished using any suitable parameter(s), including, among others, (1) hue, lightness, and saturation (e.g., for passive coding regions), (2) hue, brightness, and saturation (e.g., for active coding regions), and/or (3) dominant wavelength, luminance, and purity. Color, colored, and colorless typically are defined and/or determined in the context of a specific assay, and more particularly in the context of the specific wavelength-dependent properties and the specific detection method(s) used to detect and/or read the code in the specific assay.

The code may be a positional code where information is arrayed in ordered or unordered, spatially distinct compartments. Other positional codes may detectably alter the property of a single material at different positions, such as through changes in surface structure of the material. These changes may produce distinct optical properties of the material at these positions, for example, creating an interference filter, among others (see Examples 4 and 5).

Positional codes may be read by determining the identities, amounts, and/or other properties of the code materials at each code position, for example, by measuring intensity as a function of position. The amounts, positions, and/or values may be relative or absolute. Moreover, different types of codes may be combined to form yet other types of codes. Exemplary codes, particularly positional and/or color codes are described below in Section X.

Positional coding systems permit a code to be relatively small. Accordingly, large numbers of identifying codes on particles may be displayed efficiently in a small area. This may facilitate the use of smaller particles and smaller sample sizes. Size limitations may be particularly important for microarray experiments using costly reagents or for high-throughput applications.

The code may be positioned at any suitable location on the particle, including the entire particle or a portion or portions thereof. A code positioned only at a portion of the particle may divide the particle into at least one coding portion (or region) and at least one noncoding or assay portion (or region). Such a code may be contiguous or may include noncontiguous coding regions. The noncontiguous coding regions may include code elements that are separated by one or more noncoding portions, which may be configured to carry sample and/or reagent. The noncoding portion may be optically distinct from the coding portion and may be formed of distinct components. For example, the noncoding portion may be at least substantially colorless under the conditions of reading the code, whereas the coding portion may be colored. In some embodiments, the noncoding portion may be flanked by coding regions so that the noncoding portion is disposed at least substantially centrally, that is, in a central region of the particle. Accordingly, spaced coding regions may defined colored stripes or bands that flank the noncoding portion of a particle, so that the particle appears striped when viewed from a direction orthogonal to a line along which the code elements or code regions are arrayed.

A particle may also include orientation or alignment marks that may be used independent of the code to orient or align the particle before reading and/or interpreting the code. Suitable orientation marks include spots, crosses, and/or other shapes or patterns of shapes disposed at defined positions on the particle relative to the coding and/or noncoding portions. Such orientation marks may be used to provide a reading direction and/or starting point for reading the code. Alternatively, or in addition, particles, code elements, and/or codes may be configured so that a suitable reading direction is determinable readily. For example, particles or code elements may be shaped asymmetrically. In some embodiments, distinct codes read by starting at opposite ends of a code may be considered as equivalent.

The code also may be positioned at any suitable location relative to the samples/reagents used in the assay. Thus, the code and sample/reagent may be positioned at nonoverlapping locations on the particle (including opposite sides), at overlapping locations on the particle, or at coextensive locations on the particle.

In some embodiments, the code on a particle may be more permanent because the code may be defined at least substantially internally, between opposing surfaces of the particle. Therefore, the code may be protected within the particle from changes as a result of chemical synthesis, processing, handling, or mechanical stress, and in many cases, thermal stress and degradation by exposure to electromagnetic radiation, such as ultraviolet light. Exemplary internal codes are included below in Examples 1-3 and 8, among others.

Codes and coded particles may be described in other ways. In some embodiments, each of the particles may be formed of N separate layers, each layer having one of M different color indicia. Alternatively, or in addition, each particle may have a surface that is partitioned into N surface regions, with each region containing one of at least two different surface indicia. In some embodiments, each particle may be formed of N separate layers or bundled fibers, each layer or bundled fiber having one of M different color indicia, the layers or bundled fibers forming spatial code compartments.

Optical codes may benefit from the availability of optically distinct coding materials. Coding materials are produced in a wide array of colors, optical characteristics, and combinations of colors and optical characteristics. Consequently, greater information content may be achieved with fewer coding positions using these coding materials as compared to traditional binary bar code formats. In some embodiments, each code indicium has a different optical or spectral signature. Accordingly, detection resolution may be increased since a detector relies at least partially on spectral character rather that just intensity, as in standard uniform product code bar code reader systems, or optical encoding, as in compact disk storage systems.

In some embodiments, other coding indicia may be used alternatively, or in addition to, optical coding indicia. Such other coding indicia may include electrical properties, magnetic properties, hydrodynamic properties, functional properties, and/or temporal properties, among others. Exemplary magnetic properties include paramagnetic character, magnetic field strength, field orientation, and/or the like. Exemplary electrical properties include resistance or capacitance, among others. Exemplary hydrodynamic properties include sedimentation rate, buoyant density, sedimentation orientation, etc. Exemplary functional properties include chemical reactivity, molecular recognition, isoelectric characteristics, agglutination, surface labeling, etc. Exemplary temporal properties include time-dependent properties, such as those produced by short-pulse excitation of fluorophores having different hysteresis so that individual fluorophores emit light at different times for different durations.

Further aspects of particle codes are described elsewhere in this Detailed Description and in the patent applications identified above under Cross-References and incorporated herein by reference, particularly U.S. patent application Ser. No. 09/694,017, filed Oct. 19, 2000; and U.S. patent application Ser. No. 10/120,900, filed Apr. 10, 2002.

II. Particle Size

Particles generally comprise any structure capable of associating a sample and/or reagent with a code for a nonpositional and/or positional assay. The particles may have any suitable size consistent with an ability to perform their intended function. In some embodiments, relative particle size or the relative size of a code element within the particle may at least partially define the code.

Particle size may be selected based on competing considerations related to sample/reagent properties, the manipulability of the particles, and the nature of the assay, among others. Larger particles generally have a greater capacity for sample/reagent, and thus may be more effective for analyzing larger samples, such as cells that rely on a community effect from nearby cells for particle association or normal phenotypic behavior. Moreover, particles typically must be at least as large as the molecules, cells, or other components that they support. However, smaller particles may be more efficient in some aspects related to particle handling and distribution in liquid. Specifically, smaller particles may be resuspended more readily from a resting position in a container and may settle more slowly when suspended. Furthermore, smaller particles may be transferred more easily as a suspension in liquid, for example, using a pipette. However, in some cases, such as analyses that include repeated washing steps, rapid particle settling and less efficient resuspension may be desirable properties of larger particles. Moreover, in other cases, such as optical analyses, the particles preferably are larger than the wavelength of light but smaller than the field of view. Therefore, particle size may be adjusted to an effective balance between these competing considerations based on the specific application.

In some embodiments, particles for multiplexed experiments are small, referred to as microparticles or microcarriers, typically in the range of about 10 microns to about 4 millimeters in length or diameter. One particularly preferred particle dimension is about 360 microns by 500 microns. Numerous applications of the invention may be carried out in microplates that have a density of 96, 384, or 1536 wells per microplate. When carrying out a multiplexed experiment in a microplate well, the microparticles may be small enough so that at least two or more microparticles may be viewed side-by-side in the well simultaneously. Therefore, the maximum size dimension for microparticles may sometimes be dictated by the well dimension in a specific microplate configuration or density, with the microparticle having a diameter that is less than half the diameter of a microplate well diameter. On the lower end of the range, microcarriers for use with cells should be large enough to support at least one cell. Therefore, microparticles for multiplexed cellular experiments usually have an area of at least about 100 microns.

In some embodiments, particles may be smaller than 10 microns, for example, when the particles are associated with individual cells, for example, by attachment to the cell surface or by cell internalization, to mark the cells. These particles, termed nanoparticles, may be about 100 nanometers to 10 microns in diameter or length.

Further aspects of sizes of coded particles are described elsewhere in this Detailed Description and in the patent applications listed above under Cross-References and incorporated herein by reference, particularly U.S. patent application Ser. No. 09/694,077, filed Oct. 19, 2000.

III. Particle Shape

Coded particles may have any suitable shape that allows the particles to fulfill their intended function. Suitable shapes may be based at least partially on the type of sample, type of assay, method of particle manipulation, and/or method of reading codes and measuring sample characteristics. Preferred shapes include generally planar, for example, in the form of a flattened parallelepiped, as in FIG. 1, and at least substantially cylindrical or spherical. Here, generally planar relates to the overall shape of a particle, without considering local surface variations such as projections or recesses. The shape of a particle, or an aspect of the shape, such as the aspect ratio, may form some or all of a particle code. Alternatively, or in addition, the shape of a portion of a particle, such as a code element or a surface, may form at least a part of the code. Further aspects of particle surfaces are described below in Sections V and X, among others.

Particles with flat or planar surfaces, such as a parallelepiped-shaped particles, may be more suitable for static detection on a flat substrate or reading surface, for example, with the particles arranged on a microscope slide or on the horizontal surface of a microplate well, among others. Flat surfaces of the particle may act to self-orient the particle into contact with the assay or reading surface. In some embodiments, such particles may be thin, in the form of sheets, with an aspect ratio in which length and width of the particle are at least two-fold greater than the particle thickness. Such particles are defined as generally planar. Because they are thin, these generally planar particles may be preferentially oriented with opposing larger surfaces facing toward and away from the flat substrate or reading surface.

Cylindrical particles may be more suitable for flow-based or static detection, based on the aspect ratio of the cylinders. Elongate cylinders, having a length measured along the cylinder axis that is greater than the cylinder diameter, may be oriented, for example, by flow within a capillary tube. By contrast, shortened cylinders, in the form of discs, may be more suitably analyzed on a flat surface, as described above for planar particles.

Spherical particles may be analyzed either statically or in a flow-based system.

Other particle shapes may be suitable including cubical, pyramidal, polyhedral, ovalloid, and/or the like. The particles (and/or code elements) may have a cross-sectional shape selected from the group consisting of circles, polygons, ovals, ellipses, symbols, etc.

In some embodiments, at least one particle may be embedded in a larger spherical structure, where the code is readable from an external surface of the spherical structure. The spherical structure may be adapted to hold compounds, cells, reagents, and/or other biological materials.

Further aspects of shapes of coded particles are described elsewhere in this Detailed Description and in the patent applications identified above under Cross-References and incorporated herein by reference, particularly U.S. patent application Ser. No. 09/694,077, filed Oct. 19, 2000.

IV. Particle Materials

Particle composition may be determined by an interplay of competing considerations, such as the considerations described above for particle codes, sizes, and shapes. Preferred materials may include glass, sol-gels, ceramics, composites, plastic, film, metal, biological materials, molecular imprinted polymers, and/or combinations of these and/or other materials, including solids and/or gels, as described below.

Particles may be made from glass, as described, for example, in Example 1. Glass particles may be suitable for binding many types of samples/reagents directly, without modification, because glass is hydrophilic and thus readily wetted. In addition, many types of glass show little absorbance or autofluorescence at visible and ultraviolet wavelengths that are typically used in optical assays. Exemplary glasses include soda lime and borosilicate glass, among others.

In some embodiments, particles may be made from plastic. Suitable plastics may include any plastic that is experimentally compatible with the samples/reagents used for an assay. Exemplary plastics include, but are not limited to styrenes, polycarbonates, and acrylates, particularly methacrylates such as polymethylmethacrylates (such as PMMA), and polyethylmethacrylates (such as PEMA). Some plastic particles may be less suitable than glass for binding cells or extracellular matrix material because some plastics are hydrophobic. However, such plastic particles may be rendered suitable for binding by an appropriate treatment. For example, plastics such as polystyrene can be derivatized by irradiation, chemical modification, or other methods to provide a more hydrophilic attachment surface. When cells are analyzed by fluorescence, fluorescence emission of some plastics may interfere with sample analysis. However, low-fluorescence plastic may be suitable for such an analysis. Exemplary materials include PERMANOX (Nalge Nunc International) or methacrylates, among others, for both cell association and fluorescence measurements. The components of a plastic that would affect fluorescence measurements are known generally by those skilled in the art.

Particles may be made from other suitable materials. For example, particles may be produced from film, such as standard photographic film, as described below in Example 3. Alternatively, or in addition, particles may be produced from, or may at least partially include, molecular imprinted materials, as described below in Example 6.

Particles, or portions thereof, such as an outer layer or an internal region, may be made from a gel. For example, in some embodiments, a gel coating may provide a suitable adhesion layer for cells, and an inner gel portion may carry sample and/or reagent, or may provide for better storage or handling characteristics. Exemplary materials include gelatin, agarose, polyacrylamide, and/or any other suitable gel-forming material.

Further aspects of materials for coded particles are described elsewhere in this Detailed Description and in the patent applications identified above under Cross-References and incorporated herein by reference, particularly U.S. patent application Ser. No. 09/694,077, filed Oct. 19, 2000; and U.S. patent application Ser. No. 10/120,900, filed Apr. 10, 2002.

V. Particle Surfaces

Particles may include any suitable surface and/or surface structure as is appropriate for an assay. The surface and/or surface structure may form at least a portion of the code, may facilitate sample/reagent association and/or retention, may facilitate particle manipulation during an assay, and/or the like. The surface may include any distinct contour or surface relief feature, chemical difference (localized or nonlocalized), texture, and/or the like.

The particle surface may be modified to include surface relief. Features that define surface relief include any local deviation from a flat or convexly contoured surface, generally on an exterior surface of a particle. Exemplary surface relief features includes recesses or projections in the form of grooves, ridges, holes, bumps, depressions, dimples, and/or the like. The surface relief features may be molded, stamped, etched, cut, added by fusion, and/or the like. Such surface relief features may form a surface-contour code by modifying a property of incident light, as described in more detail in Examples 4 and 5 below. Alternatively, or in addition, the surface relief features may facilitate sample attachment, retention, and/or accessibility of reagent/sample to the particle surface, among others, as described in more detail below in Example 1.

The surface (or a region thereof) may be chemically distinct relative to interior portions of the particle. Such a chemically distinct surface may be formed by chemical reaction with the surface and/or attachment of a distinct material. The distinct material may be a film, an applied material or mixture (such as a cell-derived mixture), and/or other coating.

Glass may be modified readily at its surface to promote sample/reagent attachment, optical analysis, code formation, and/or the like. Exemplary adhesion promoters include aminosilane; polylysine; gelatin; atelocollagen; polyethylenimine; a dendrimer, such as a cationic or amphipathic dendrimer (for example, an activated-dendrimet available from QIAGEN); an extracellular matrix component, mixture, or extract; serum albumin; a nucleic acid binding protein or other macromolecule, including sequence-specific or —nonspecific DNA and/or RNA binding proteins; compounds that bind nucleic acids, such as intercalating agents (ethidium monoazide, ethidium bromide, etc.) or agents that bind to the major or minor groove of a nucleic acid duplex; nucleic acids, such as single- or double-stranded DNA or RNA that form hydrogen bonds with the transfection material; and/or the like. Exemplary materials that impart an optical property to the surface may include dyes, reflective metals, light-polarizing oriented polymers, and/or other optically active materials.

Plastic particles may have a surface(s) or a surface region(s) with modified chemistry. For example, PEMA particles may be modified chemically with allyl amine, ammonia, or carbon dioxide, among others. Alternatively, or in addition, plastic particles or particles formed of any other material may be modified with any of the materials described above for modifying the surface of glass particles or described elsewhere in this Detailed Description.

Surface modifications that facilitate sample/reagent association or described in more detail below in Section VIII. Further aspects of particle surfaces are described elsewhere in this Detailed Description and in the patent applications identified above under Cross-References and incorporated herein by reference, particularly U.S. patent application Ser. No. 09/694,077, filed Oct. 19, 2000; and U.S. patent application Ser. No. 10/120,900, filed Apr. 10, 2002.

VI. Particle Manipulation

Particles may be manipulated based on structural features and/or materials included in the particles. Manipulation may include selected rotational and/or translational movement of individual particles or groups of particles.

Structural features that may facilitate particle manipulation include size and shape. As described in Sections II and III, size and shape may determine hydrodynamic properties of particles, so that particles self-orient as they settle out of fluid onto a surface. Alternatively, or in addition, particles may include microscopic “handles” by which individual particles may be manipulated by contact between a micromanipulator and the particles.

In some embodiments, particles may include materials or shapes that respond to an applied force. The particle may be attracted or repelled by gravity, electrostatic forces, electrophoretic forces, dielectric forces, light, and/or magnetism, among others. The materials may be localized within and/or on the particles. Suitable materials may include magnetic materials (paramagnetic and/or ferromagnetic materials), electrically conductive materials, and/or materials of different densities. Force-responding materials may be used to orient the particles and/or to move the particles translationally, for example, to separate or sort the particles.

In some embodiments, particles may include a shape with a flat “viewing surface” that would self-orient such that at least substantially all particles in an array settle with the viewing surfaces aligned parallel Accordingly, disks and/or planar particles may be preferred because these particles may self-orient with a flat surface facing upwards. This may be helpful when the code is viewable from the flat surface, for example, with strands of colored fibers in a bundle that is later sliced to form disks, rods, or, sheets. Alternatively, or in addition, weight distribution within the particle may facilitate orientation. For example, hemispheres may settle in a fluid with their flat surface upwards if the hemisphere is weighted on the apex of the spherical side.

Particle orientation may be important when particle codes are read. With codes elements arrayed along a line or plane, coding regions should be exposed so that they can be viewed from a direction generally orthogonal to the line or plane. As discussed above, orientation may be specified by physical properties of the particles. Orientation may be specified by particle shape, but it may also be specified by density and/or weight distribution. Particles may be oriented further by the application of an external force aside from gravity. For example, particles may have a paramagnetic quality such that when they are in the presence of a sufficiently strong magnetic field, they will align themselves accordingly. In some embodiments, particles may demonstrate dielectric potential such that particles may be daisy-chained by the application of a dielectrophoretic alternating current.

Further aspects of particle manipulation and manipulation features are described elsewhere in this Detailed Description, such as below in Example 1 of Section X, and in the patent applications identified above under Cross-References and incorporated herein by reference, particularly U.S. patent application Ser. No. 09/694,077, filed Oct. 19, 2000; and U.S. patent application Ser. No. 10/120,900, filed Apr. 10, 2002.

VII. Particle Manufacture

Particles may be manufactured by any suitable process based on the materials, type of code, number of code elements or component pieces, etc. Particles may be formed in one step or in a sequence of steps. Particles may be manufactured unitarily from one component, for example, as described, for example, in Examples 4 and 5. Alternatively, particles may be manufactured or as a composite of plural structural components, as described, for example, in Examples 1 and 2.

The code may be formed before, during, and/or after particle manufacture. In some embodiments, structural components of the particle define code elements (see Examples 1 and 2). Some or all of the structural components that form a particle may have one of plural optical properties or indicia. The optical properties may be formed by introducing an optically responsive material, such as a dye (for example, a fluorophore, chromophore, etc.), into the particles or into particle components during their manufacture. Alternatively, or in addition, the optically responsive properties may be added later by application of an optically responsive material to the particle surface, by chemical reaction in situ, and/or the like. In some embodiments, the code may be formed integrally with the particle, for example, being defined by some or all of the structural components of the particle (see, for example, Examples 1, 4, and 5 below).

In some embodiments, nanocrystals may be used as optically responsive materials that form codes. Nanocrystals may extend the number of distinctly detectable optical properties by producing narrower emission spectra. Accordingly, nanocrystals may be used as indicia to provide narrow bandwidth emission for low optical spill-over detection between coding regions of a spatial code. For example, the nanocrystals may be embedded in the matrix of a fiber, filament, or layer, among others, to form an optically responsive structural component of a particle, such as a fiber that is bundled with other fibers and then sectioned into plural coded particles. Some embodiments of coded particles may incorporate 3 nm CdSe nanocrystals and/or 4.3 nm InP nanocrystals at coding positions within a particle. Using UV light excitation, or any wavelength below the emission peak of the highest energy emitting crystal in use, the fluorescence of these two different classes of crystal may be detected and their relative positions recorded. In another manifestation using time-gated detection, the fluorescence lifetime may be recorded, which may help with eliminating autofluorescence and background.

Particles may be produced from a progenitor structure, such as a bundle or assembly of discrete structural components. The progenitor structure may be modified, for example, to attach the components to one another and/or to change the dimensions of the structure, and then the structure may be cut into two or more particles. The progenitor structure may be configured with code elements (and thus the code) arrayed generally along a line or plane, so that the progenitor structure can be cut normal to the line or plane without destroying coding information. For example, particles may combine or bundle together several different strand materials. Strands may differ by optical property (such as color, refractive index, shade), response to chemical treatment, physical property including magnetism, and/or by composition, among others. Bundled strands then may be pulled and stretched to reduce the diameter of the bundle. Heat and/or pressure may be applied to promote attachment of the strands to one another and to enable the stretching process. In some embodiments, the aspect ratio of the bundle is not changed as the bundle is stretched. Once a desired diameter is attained, the bundles may then be cut mechanically, optically, and/or chemically, that is sliced, sheared, or abraded, among others, to produce particles shaped as sheets, wafers, rods, disks, elongate cylinders, or the like. Longer segments may be cut to produce rods that may be read by rotating the rod while observing the circumference of the rod-cylinder. Particularly preferred methods are described in U.S. Pat. Nos. 4,640,035; 4,390,452; 4,329,393; 4,053,433; and 3,897,284, each of which is incorporated herein by reference. Producing particles by stretching may offer advantages over standard lithographic technologies because miniaturization may be achieved by stretching the length of fibers to reduce the cross-sectional diameter of fiber bundles containing the fibers. In some embodiments, a surface of the progenitor structure may be shaped so that a surface of the structure includes surface relief. This shaping process may be performed one or more distinct components of the progenitor structure, either before, during, and/or after assembling the components of the progenitor structure. The surface relief may be disposed so that it is divided among progeny particles when the progenitor structure is cut into particles. Further aspects of forming particles by stretching and then cutting a progenitor structure are included below, for example, in Examples 1 and 2.

Films may be used for particles. In particular, U.S. Pat. No. 4,390,452, which is incorporated herein by reference, relates to the use of microfilm or microfiche disks or fragments, photographically imprinted with a code to create taggants. Films may be layered further upon an orienting layer to aid in orienting the image for visualization. Films also may be patterned by inkjet, photolithographic, electrostatic, or xerographic methods. Use of films to form coded particles is described in more detail below, for example, in Example 3.

In some embodiments, a thin-film layer may be formed on a surface of the particle. The thin-film layer may be restricted to a portion of the surface, for example, a patterned film, and/or may have a uniform optical property or optical properties that vary across the film layer.

In some embodiments, a cuboidal particle may be made from a first set of layers sandwiched together to form a cross-sectional code, and then flanked on opposing sides with yet another sandwich code so that every face of a sectioned portion of the particle displays the sandwich sequence or code.

Milifiori or milli fiori glass manipulating techniques may be employed to impart a distinguishing shape upon the cross section of each coding region. For example, a star cross-section fiber may be fused with a cladding to form a particle having a calorimetric identifier or other optical identifier, and a spatial code of a star shape. Such coding may impart a third level of coding to the particle, that is, a shape within a shape. Other shapes may be used, preferably in combination with other shaped fibers.

Patterns may also be formed in microchips by photolithography, or onto films such as with microfilm technology. For example, shapes may be combined with colors to improve diversity. Available indicia from one class may be combined with indicia from other classes to further broaden the coding vocabulary. Layers may be formed, for example, as sandwiches, ribbons, twines, ropes, concentric spheres, cables, strands, cylinders, cubes, disks, pyramids, or combinations thereof. Further aspects of forming particles using photolithography are described below in Example 2.

Particles may also be composed of plastics and shaped by processes such as injection molding or extrusion, or from other substrates by micro-engineering processes (e.g.—MEMS —micro-electric mechanical systems) familiar to those skilled in the art. Micro-injection molding of small detailed parts such as micro-gears is commercially available. Using this method, the identifying indicia incorporated into particles may include the formation of various shapes or lines, and/or the location of processes about the periphery of the particle, such as the specific relative placement of “gear teeth” about a gear. Similarly, the use of plastics such as polymethylacrylates in plastic optical fibers makes possible methods of producing particles analogous to that used to prepare particles from glass fibers.

Further aspects of fabricating coded particles are described elsewhere in this Detailed Description, such as Examples 1-8 of Section X, and in the patent applications identified above under Cross-References and incorporated herein by reference, particularly U.S. patent application Ser. No. 09/694,077, filed Oct. 19, 2000; and U.S. patent application Ser. No. 10/120,900, filed Apr. 10, 2002.

VIII. Associating Samples/Reagents with Particles

Each particle may be associated with at least one sample or reagent, thus linking a code on the particle to the sample/reagent. The association between sample/reagent and a particle generally comprises any relationship between the particle and the sample/reagent that maintains physical proximity of the sample/reagent and particle (and thus code) during an analysis. Association may include attaching a sample and/or reagent to a particle so that the particle is a carrier that supports or holds the sample and/or reagent. In this case, the code of each particle identifies the attached sample/reagent. Alternatively, or in addition, the particle may be physically proximate to the sample(s) and/or reagent(s) during an analysis without sample/reagent attachment. For example, the particle may be internalized by a sample, such as uptake of the particle by a cell, attached to the surface of a sample, or the particle may be proximate to or contained in a compartment that holds or carries sample and/or reagent. If this proximity is maintained during the analysis, the particle code may be used to identify the sample/ reagent. Exemplary compartments that may be identified by a coded particle include microplate wells, test tubes, regions of a substrate, and/or the like.

Samples and reagents may be used to perform multiplexed analysis with coded particles. As used herein, the term “sample” refers to any material of interest that is analyzed in a multiplexed assay. Samples may include cells, viruses, proteins, nucleic acids, carbohydrates, extracts, lysates, secretions, clinical samples, tissue biopsies, environmental samples, receptors, ligands, plasmids, synthetic compounds, natural compounds, and/or so on. As used herein, “reagent” refers to any material that contacts a sample to produce or facilitate production of a measurable experimental result. Exemplary reagents include ligands, receptors, small molecules, hormones, proteins, nucleic acids, viruses, cells, enzymes, dyes, lipids, carbohydrates, reaction mixtures, and/or the like. Additional samples and reagents, particularly cell-based samples and cell-analysis materials that act as reagents, are described in more detail in the patent applications identified above under Cross-References and incorporated herein by reference, particularly U.S. patent application Ser. No. 09/694,077, filed Oct. 19, 2000; and U.S. patent application Ser. No. 10/120,900, filed Apr. 10, 2002.

A group of one or more samples and/or one or more reagents may be associated with a particle to produce a particle assembly. This association may be direct or indirect, and may include linkage, attachment, or adhesion. Binding may be mediated by any suitable mechanism, including electrostatic interactions, covalent bonding, ionic bonding, hydrogen bonding, van der Waals interactions, and/or hydrophobic-hydrophilic interactions, among others. In general, binding may be facilitated by the appropriate selection, treatment, and/or modification of the particle, sample, reagent, or a suitable combination thereof.

Binding may be facilitated by appropriate selection of the particle material, geometry, and association region, for example, as described above in Sections III-V and below in Section X, particularly Example 1. Samples/reagents may associate with external or internal regions of particles. Thus, particles may include a relatively flat or gently contoured external binding surface, or such a surface modified to include surface relief structure, so that cellular samples may bind. Moreover, particles may include a modifiable binding surface, so that the surface may be treated or composed (for example, using a sol-gel) as desired to promote binding of samples/reagents. Alternatively, the particle may be a porous material, such as a gel or porous polymer, which allows samples/reagents to migrate inside or to be directly included in the interior of the particle.

Binding may be facilitated by appropriate treatment of the particles, either before or after combination with sample/reagent. Suitable treatments may include chemical reaction, charge modification, temperature changes, light exposure, radiation exposure, and/or desiccation, among others. Thus, in some applications, the particle surface may be pretreated or otherwise modified so that electrostatic or, in given cases, van der Waals or covalent binding of sample/reagent is promoted. For example, the binding surface may be coated with an adhesion promoter, such as poly-L-lysine, poly-D-lysine, gelatin, collagen, laminin, fibronectin, proteoglycans, polyethylenimine, albumen, BIOMATRIX EHS (Nunc Nalge International), BIOBOND (Electron Microscopy Services, Inc.), CELL-TAK, and/or MATRIGEL (both from Becton-Dickinson), or an extract from a cell, tissue, or embryo, among others. Alternatively, cellular samples may adhere indirectly through an associated layer of cells, for example, fibroblasts used to culture embryonic stem cells. Alternatively, or in addition, the binding surface may be modified in a way that promotes molecule- or cell-specific binding, such as with avidin and/or biotin, or by modification with immobilized lectins.

In some cases, association of samples/reagents with particles may be facilitated by interactions between specific binding pairs (SBPs), where one member of the pair is associated with the sample/reagent and the other member of the pair is associated with the particle. The interactions between members of a specific binding pair typically are noncovalent, and the interactions may be readily reversible or essentially irreversible. An exemplary list of suitable specific binding pairs is shown below in Table 1.

TABLE 1
Representative Specific Binding Pairs

| First SBP Member | Second SBP Member |
|---|---|
| antigen | antibody |
| biotin | avidin or streptavidin |
| carbohydrate | lectin or carbohydrate receptor |
| DNA | antisense DNA; protein |
| enzyme substrate | enzyme; protein |
| histidine | NTA (nitrilotriacetic acid) |
| IgG | protein A or protein G |
| RNA | antisense or other RNA; protein |

Association also may be facilitated by appropriate selection and/or treatment of the medium in which the sample/reagent and particles are combined. For example, the medium may include binding mediators that participate in or otherwise promote interactions between sample/reagent and particles, for example, by forming cross-bridges between samples and particles and/or by counteracting the effects of binding inhibitors associated with the sample/reagent and/or particles. The binding mediators may act specifically, for example, by binding to specific groups or molecules on samples/reagents and/or particles. Thus, biotin might act as a specific binding mediator by binding to and cross-linking avidin or streptavidin on samples/reagents and particles. The binding mediators also may act less specifically, or nonspecifically, for example, by binding to classes or categories of groups or molecules on the samples/reagents and particles. Thus, Ca²⁺ ions might act as a relatively nonspecific binding mediator by binding to and cross-linking negative charges on samples/reagents and particles.

Association of sample/reagent may occur indirectly with the particle (or treated particle). Thus, association may occur via interaction with other sample/reagent also associated with the particles. For example, indirect association of a sample with a particle may be mediated by an attached reagent, for example, by binding of a cellular sample to a cellular ligand (or candidate ligand) that has been pre-associated with the particle Sample association may facilitate subsequent analysis of the sample. Alternatively, the presence, absence, or level of association or binding of sample (or reagent) to a particle through a reagent (or sample) may provide an experimental result.

Association of cellular samples and particles, or subsequent analysis of the cells, may be promoted or facilitated in some embodiments by fixing the cells. This procedure typically kills cells and may lock macromolecules into stable configurations, in some cases by creating covalent bridges between macromolecules or by denaturing them. Any suitable fixative may be used, including (1) aldehydes, such as paraformaldehyde or glutaraldehyde, (2) alcohols or other organic solvents, such as methanol, ethanol, isopropanol, or acetone, (3) oxidative agents, (4) mercurials, and/or (5) picrates. Cells may be fixed before, during, and/or after being associated with particles, or they may remain unfixed.

Samples/reagents may be distributed on or placed in association with particles by any suitable method. In some embodiments, samples/reagents may be mixed with particles, allowing the samples/reagents to associate with all available portions of the particles. In other embodiments, association may be at least substantially restricted to one or several surfaces of the particles or regions within a surface(s). The samples/reagents may be combined with the particles so that the samples/reagents selectively encounter and thus associate with a portion of the particle. For example, particles may be distributed randomly, but substantially in a monolayer, on a horizontal surface, such as the bottom of a tissue culture container. Cells in suspension may be added to the container and allowed to settle onto an upwardly facing surface of the particles.

Association of samples/reagents with particles also may occur with the particles provided in a positional array, for example, by arranging or forming the particles on a substrate. Individual samples/reagents may be disposed on particles within the array, or a single sample or reagent may be combined with and allowed to associate with the array, for example, on an accessible face of the array. After association between samples/reagents and particles in the array, particle distribution may be randomized to produce nonpositional arrays by removing the particles from the positional array. Association of sample with particles distributed in an array may allow a more economical use of samples that are available in limited quantity, for example, from a patient sample.

Further aspects of associating samples/reagents with particles are described elsewhere in this Detailed Description and in the patent applications identified above under Cross-References and incorporated herein by reference, particularly U.S. patent application Ser. No. 09/694,077, filed Oct. 19, 2000; U.S. patent application Ser. No. 10/120,900, filed Apr. 10, 2002; and U.S. Provisional Patent Application Serial No. 60/413,675.

IX. Sample Analysis using Coded Particles

Sample may be analyzed for any suitable sample characteristics, based on the type of sample(s) and reagent(s) and the experimental procedure carried out. Exemplary sample characteristics include presence/absence/level of an analyte, of an interaction between the sample and a reagent, of a cellular material, and/or of a cellular phenotype, among others. Sample assays that may be conducted in a multiplexed format with coded particles, particularly assays with cell-based samples, are described in more detail in the patent applications identified above under Cross-References and incorporated herein by reference, particularly U.S. patent application Ser. No. 09/694,077, filed Oct. 19, 2000; and U.S. patent application Ser. No. 10/120,900, filed Apr. 10, 2002.

A characteristic of a sample may be measured, and the code of the associated particle may be read, before, during, and/or after an assay procedure on the sample. The steps of reading and measuring generally may be performed in any order, and each step may be performed selectively on specific particles. For example, in some cases the code may be read only on particles that exhibit a specific sample characteristic, such as showing a positive signal. Alternatively, the sample characteristic may be measured only for particles that have a specific code(s) among particles in an array. Moreover, these steps may be performed using any suitable substrate, such as a slide, a microplate, or a capillary tube, among others, and any suitable detection device, such as a microscope, a film scanner, or a plate reader, among others.

Codes, sample characteristics, and other measured quantities may be determined using any suitable measurement method. The measured quantities generally comprise any measurable, countable, and/or comparable property or aspect of interest. The detection methods may include spectroscopic, hydrodynamic, and imaging methods, among others, especially those adaptable to high-throughput analysis of multiple samples. The detection methods also may include visual analysis. Measured quantities may be reported quantitatively and/or qualitatively, as appropriate. Measured quantities may include presence or absence, or relative and/or absolute amounts, among others.

Spectroscopic methods generally involve interaction of electromagnetic radiation (light or wavelike particles) with matter, and may involve monitoring some property of the electromagnetic radiation that is changed due to the interaction. Exemplary spectroscopic methods include absorption, luminescence (including photoluminescence, chemiluminescence, and electrochemiluminescence), magnetic resonance (including nuclear and electron spin resonance), scattering (including light scattering, electron scattering, and neutron scattering), circular dichroism, diffraction, and optical rotation, among others. Exemplary photoluminescence methods include fluorescence intensity (FLINT), fluorescence polarization (FP), fluorescence resonance energy transfer (FRET), fluorescence lifetime (FLT), total internal reflection fluorescence (TIRF), fluorescence correlation spectroscopy (FCS), and fluorescence recovery after photobleaching (FRAP), their phosphorescence analogs, and bioluminescence resonance energy transfer (BRET), among others.

The same and/or different spectroscopic methods may be used to read the code and measure cell characteristics. For example, both the code and a sample characteristic may be detected through absorption of electromagnetic radiation, such as visible light. Particles with distinct coding and noncoding portions, such as those described in Section X, particularly Example 1, may be suitable for analysis with a single spectroscopic method. Alternatively, or in addition, the code and cell characteristics may be measured with different spectroscopic methods and/or detection methods.

Hydrodynamic methods generally involve interaction of a molecule or other material with its neighbors, its solvent, and/or a matrix, and may be used to characterize molecular size and/or shape, or to separate a sample into its components. Exemplary hydrodynamic methods may include chromatography, sedimentation, viscometry, and electrophoresis, among others.

Imaging methods generally involve visualizing a sample or its components. Exemplary imaging methods include optical microscopy and electron microscopy, among others. Exemplary imaging data include analog and digital images, among others.

Exemplary methods for reading codes and measuring sample characteristics are described in more detail elsewhere in this Detailed Description and in the patent applications identified above under Cross-References and incorporated herein by reference, particularly U.S. patent application Ser. No. 09/694,077, filed Oct. 19, 2000; U.S. patent application Ser. No. 10/120,900, filed Apr. 10, 2002; and U.S. Provisional Patent Application Serial No. 60/348,025, filed Oct. 26, 2001.

### X. EXAMPLES

The following examples describe selected aspects and embodiments of the invention, including methods for making and using coded particles. These examples are included for illustration and are not intended to limit or define the entire scope of the invention. The examples include (1) coded particles with coding and noncoding portions, surface relief features, and/or magnetic portions, (2) coded particle embodiments, (3) film-based coded particles, (4) particles with surface-contour codes, (5) particles with topographic codes, (6) particles utilizing molecular imprinted materials, (7) multiplexed analysis using chromic materials, and (8) coded particles with metal features.

### Example 1

### Coded Particles with Coding and Noncoding Portions, Surface Relief Features, and/or Magnetic Portions

This example describes coded particles having distinct coding and noncoding portions, surface relief features, and/or magnetic portions for use in nonpositional and/or positional arrays; see FIGS. 1 and 3-12.

Particles, such as microparticles, have numerous uses as fillers, tracers, carriers, or tags. For example, particles may be useful as identifying labels to track a material and/or to mark the material for future identification. The general usefulness of particles stems in part from their small size, which may render individual particles unobtrusive or completely invisible to the unaided eye. In addition, small particles may be readily manipulable, for example, in a fluid environment.

Despite the many uses of particles, their small size may limit effective use in biological assays as coded microparticles to track biological samples. In particular, the small size of particles tends to reduce the available surface area for attaching cells and/or reagents, thus limiting the amount of sample that can be analyzed on one particle. Thus, the sample may be disposed over the code, so that the code interferes optically with sample analysis. Alternatively, the sample may be spaced from the code. However, a spaced sample may create difficulties in assigning linkage relationships between the sample and the code. For example, in higher density distributions of particles, codes from two or more particles may be disposed near the sample, and more than one sample may be disposed near each code. Accordingly, some samples may be identified incorrectly as a result of incorrect assignment of which codes are linked to which samples. Furthermore, some samples may need to be ignored in an analysis when the sample-code linkage is ambiguous, thereby increasing the number of assays that should be performed. Therefore, coded particles are needed that define code-sample linkages more efficiently and accurately during optical analysis, while allowing analysis of samples and codes at discrete portions of the particles.

Small particles may create additional problems. For example, the surfaces of particles, particularly flat or convex surfaces, may not be sufficiently effective at holding some samples, such as some types of cells, during experimental manipulations. Furthermore, the small size of particles may render the particles difficult to sort or separate after analysis, for example, to isolate or purify cells, cell components, and/or cell-analysis materials (reagents) that are bound during the analysis. Thus, a coded particle having a small overall size but with surface structure that improves cell retention would be useful. Furthermore, a coded particle that is easily manipulated magnetically during or after an analysis also would be useful.

Coded particles having distinct coding and noncoding portions are provided. The distinct coding portion of each particle may include one coding region or a plurality of spaced coding regions that define the code. The coding portion may at least partially frame or border the noncoding portion. Accordingly, the noncoding portion may occupy a generally central position of the particle and may be flanked by the coding regions on at least one or both of two opposing sides of the noncoding portion. The noncoding portion may be colorless, and the coding portion may be colored. Accordingly, the position of the noncoding portion and its associated sample/reagent may be more clearly defined, because the coding and noncoding portions contrast optically.

Coded particles having surface relief also are provided. The surface relief may include any deviation from a flat or convexly contoured surface to form a projecting or recessed region of the surface. Recessed surface regions may provide advantages over flat and/or continuously convex surfaces. For example, the recessed regions may retain sample more effectively by, for example, providing a better gripping surface for the sample and/or minimizing fluid flow and/or turbulence near the surface region during particle manipulations in fluid. In some embodiments, the recessed regions are configured to at least partially receive one or more cells. Alternatively, or in addition, the recessed regions may increase the particle surface area and/or may provide more effective access of fluid (and reagents) when a surface of the particle abuts a generally complementary supporting surface, such as the flat surface provided at the bottom of a microplate well. In some embodiments, projections are included on particles. Such convex projections may increase surface area, facilitate particle manipulation, and/or provide identifying regions of the particles, among others. When used with cells, the formation of a more three-dimensional surface (ridges, etc.) may improve the probability of forming attachments to the cells, relative to a more two-dimensional surface.

Coded particles having magnetic portions are also provided. The magnetic portion may include a magnetic material attached to, embedded in, or otherwise associated with the particle and the code. Such magnetic portions may improve particle handling, sorting, orientation, and/or the like. The location and optical properties of the magnetic portion of the code may also be used as a component of the coding region.

1.1 Surface Relief

Particles are provided that have surface relief structure defined by one or more surface relief features. Surface relief generally includes any surface topography that modifies a planar surface, a polyhedral surface (such as a cube), and/or a contoured convex surface, (such as cylindrical, ovalloid, spherical, and so on). Accordingly, surface relief features may include regular or irregular recesses and/or protrusions that extend inward or outward, respectively, along at least one axis or radius relative to adjacent particle surfaces. Exemplary surface relief features may include grooves, ridges, dimples, bumps, through-holes, pockets, projections, ripples, and/or flaps, among others, and may have any suitable shape and size. The surface relief features may be regularly or irregularly spaced and/or sized. The surface relief features may be included on one or more surfaces or sides of a particle, and/or on one or more regions of a surface or side. For example, the surface relief features may be included in both coding and noncoding portions of a particle, or may be restricted to one of these two portions. Furthermore, the surface relief features may be different within a set of particles, for example, so that the surface relief of a particle corresponds to or at least partially determines the particle code. The surface relief features, alternatively or in addition, may be different on different surfaces or sides of one particle. For example, one or more surfaces or sides of a particle may provide surface relief features that form some or all of a particle code, and one or more surfaces may provide surface relief features that facilitate sample retention and/or processing. Further aspects of surface relief features that contribute to the particle code are included below in Examples 4 and 5, among others.

The surface relief features may be formed on a generally planar surface, corresponding generally to a side or a particle. For example, the surface relief features may be recesses and/or projections that modify the generally planar surface. Such surface relief features may be disposed in a regular and/or repeating pattern on the surface(s) or side(s) of the particle and may extend to be disposed near one or more edges of a surface (or side) and/or may have ends and/or positions that are spaced from the edges of the surface (or side).

1.2 Coded Particles with Recesses

The coded particles may be formed to include one or more recesses. A recess generally comprises any portion of a particle surface that is sunken or lowered relative to adjacent surface portions along an axis or radius and/or relative to the general shape of the particle. Recesses may have any desired shape. A recess may be a through-hole and thus may extend between two or more sides of a particle, such as two opposing sides, or the recess may extend into, but not through the particle. The recess may provide interior surface regions that are in fluid communication with exterior surface regions of the particle. The interior surface regions may include generally planar surface region that are parallel to exterior top and bottom surfaces of a planar particle. The recess may have a rectilinear configuration, bounded by rectangles, or any other suitable geometry, such as cylindrical, spherical, or elliptical, among others. Recesses may be used for sample and/or reagent association (or attachment), and thus may increase the available surface area for sample and/or reagent and/or may provide an area of the particle that is more shielded, for example, from fluid movement or external contact. The surface of the recess itself also may be rough or irregular.

A particle generally may include one or more recesses. These recesses may have similar sizes and/or shapes, and they may have at least generally parallel orientations relative to one another and/or the particle. For example, the recesses may originate at a side of the particle and extend to a common, opposing side of the particle to form a hole, or the recesses may be restricted to one exterior side or surface of the particle, to form, for example, a depression or groove. Alternatively, or in addition, at least some of the recesses of a particle may have different sizes, shapes, and/or orientations.

A recess may be disposed in a noncoding and/or a coding portion of the particle. The recess may be disposed in a noncoding portion, with the code formed in a spatially distinct, generally nonoverlapping portion of the particle. For example, the recess may be centrally located and flanked on both sides by coding regions. In addition, the recess may extend completely, as a through-hole, across a central portion, with code elements disposed on one or both flanking side portions of the particle. Alternatively, the recess may be disposed, at least partially, in a coding portion of the particle. For example, the recess may partially overlap a coding portion, or may be included completely in a coding portion, particularly when a coding portion forms a substantial portion of a particle.

Surface relief features, such as ridges or grooves described below, may have a depth (or height) relative to the particle surfaces that are adjacent. For example a groove may have a depth relative to adjacent nonrecessed regions (for example, ridges) of between about 0.1-200, 0.5-100, or about 1-50 microns. Alternatively, or in addition, the depth or height may be related to the average diameter of cells that the particle may be configured to carry, with groove depths (relative to adjacent ridges) of at least about one-half, one, or two or more cell diameters. Such grooves may at least partially receive one or more cells.

1.3 Coded Particles with Grooves and/or Ridges

In some embodiments, the surface relief features may include grooves and/or ridges that are formed on one or more surfaces. Grooves generally include any elongate recess with raised sides, and ridges generally include any elongate raised regions of a particle surface. The grooves and/or ridges may extend to different sides or edges of a particle or each groove/ridge may begin and end on one surface or side of the particle. Grooves are generally accompanied by ridges that are disposed between the grooves. The ridges/grooves may be similar or distinct in width. The grooves and/or ridges may be angular and/or arcuate in cross section, for example, the grooves or ridges may be rounded, flat, or angled at their bases or apexes, respectively. The grooves/ridges may be generally linear, curvilinear (such as arcuate, wavy, circular, and/or elliptical, among others), and/or bent. In some embodiments, the grooves/ridges may be restricted to noncoding portions of the particles. Wherever the grooves/ridges are positioned, they may be arrayed generally parallel to an axis or plane along which the coding elements are arrayed, so that each groove/ridge extends generally perpendicular to the coding element array. Alternatively, or in addition, the grooves/ridges be arrayed obliquely, radially, and/or perpendicular to the array defined by the coding elements. Exemplary coded particles having grooves and ridges are described in more detail below in Section 1.8.

1.4 Forming Coded Particles having Surface Relief Features

Surface relief features may be formed on coded particles by shaping a surface of the particles before, during, and/or after particle production.

A surface relief feature may be formed before particle production, for example, when the particle is manufactured as a composite of component structures. In this case, at least one of the component structures may include a preformed surface relief feature, so that joining the distinct component structures places the preformed surface relief feature on the particle.

A surface relief feature may be formed during particle production. For example, a surface relief feature may be formed by joining component structures in offset positions. Alternatively, or in addition, a surface relief feature may be formed by molding the particle to include the surface relief feature.

A surface relief feature may be formed after particle production, for example, by reshaping the particle surface. For example, a surface relief feature may be introduced with a cutting or boring device, among others. An exemplary cutting device may include an eximer laser or set of lasers. In other embodiments, a surface relief feature may be formed by chemical modifiers, alone or in combination with physical modifiers, to selectively alter or remove a portion of the particle. Physical or chemical modifiers may include etching reagents, such as acid, base, oxidizing agents, reducing agents, and the like; light; RF-irradiation-grafted materials such as polymeric material; or any other treatment that locally or globally alters the properties of the particle. A portion of the particle may be exposed locally to modifiers, for example, by using a mask or template. Alternatively, the entire particle may be exposed to a modifier, but portions of the particle may be differentially sensitive to the modifier. For example, the particle may be formed as a composite of different materials that are differentially sensitive to a treatment, such as acid-sensitive and -resistant glass. Section 1.8 describes exemplary methods of forming grooves and ridges using acid-sensitive and -resistant fibers.

1.5 Coded Particles with Magnetic Portions

A coded particle may include one or more magnetic portions. A magnetic portion generally comprises a region of the particle that is capable of being magnetized or attracted by an appropriate magnet. The magnetic portion may allow the particle to adhere to and/or be moved/rotated by a magnet. The magnetic portion may be used to separate the particle from other particles. Alternatively, the magnetic portion may be used to rotate and thus orient the particle or a group of particles for reading the code and analyzing the sample. The magnetic portion may include a premagnetized material or an inductively magnetized material. Suitable materials for the magnetic portion may include paramagnetic materials and/or any ferromagnetic materials, such as iron, nickel, and cobalt, among others.

The magnetic portions may be attached externally and/or internally and may be disposed in a discrete region of the particle or extend throughout the particle. The magnetic portions may be embedded in the particle during its formation, or they may be attached to the particle after it is formed, for example, by bonding or grafting. The magnetic portions may have any suitable configuration, including a particle, a cylinder, a sheet, a beam, a bead(s), or any other structure that provides sufficient mass relative to the particle mass to create an attractive force with an appropriate magnet. In some embodiments, the particle may be formed entirely of a material that has paramagnetic and/or ferromagnetic properties.

1.6 Particles with Coding and Noncoding Portions

FIG. 1 shows an embodiment of a particle 70 that includes both coding and noncoding portions, but lacks a recess or magnetic portion. Particle 70 includes a centrally disposed noncoding or assay portion 82 flanked by a frame or coding portion 80 that contrasts optically with noncoding portion 82. For example, frame portion 80 may be colored and noncoding portion may be at least substantially colorless under the conditions with which the assay is performed. Frame portion 80 may include plural frame regions 81a,b disposed on opposing sides of noncoding portion 82. One or more of the frame regions also may be coding regions that contribute to code 72. Here, frame regions 81a,b are shown as bands that extend along an entire side of the noncoding portion. However, the particle may include any optically contrasting frame regions disposed at any suitable position relative to the assay portion, each other, and/or the particle itself. Accordingly, frame regions may be formed as spots, lines, bars, circles, or any other suitable shapes, disposed adjacent an assay portion, within an assay portion, internal to and/or at the surface of the particle, and/or so on. The frame regions may partially or completely define the perimeter of the assay portion and/or particle. Accordingly, one or plural frame regions may act as optical landmarks or optical reference structures disposed at defined positions relative to each other, relative to the assay portion, and/or relative to the particle. The defined positions may be relative to an edge or side of a particle surface, an edge or side of an assay portion surface, and/or the like.

Noncoding or assay portion 82 has upper and lower association surfaces 86, 88 that are included in particle surface 78, for associating or attaching at least one sample, such as cells 76, and/or at least one reagent. Each surface may include a perimeter that includes or joins to generally opposing edges 90, 92 or sides of noncoding portion 82. Association surfaces 86, 88 may be at least substantially planar, or they may be generally planar but include surface relief features (see below). In some embodiments, the noncoding portion may be constructed of clear, colorless glass.

Noncoding portion 82 and particularly surfaces 86, 88 may be at least partially framed (or flanked) by coding portion 80 on one or more sides of the noncoding portion. In some embodiments, the coding portion 80 also may include plural coding regions 81a,b, each of which may be disposed adjacent or attached near or at a different opposing side or edge 90, 92 of noncoding portion 82, near a perimeter of surface 86 and/or 88. Accordingly, the coding regions may flank the noncoding portion and, due to optical contrast, may frame adjacent surfaces 86, 88 on at least two sides, for example, by delineating edges 90, 92 and thus a portion of the perimeter. Coding regions 80 thus may define two sets of spaced or noncontiguous code elements 74, with each set having one or plural code elements. Code elements 74 (and noncoding element 84) may be structural elements or components formed, for example, of glass, polymeric materials, and/or laminates. The coding regions and thus the code elements may contrast optically with the noncoding portion. For example, code elements 74 may be colored using optical limiting agents, which determine the absorption or reflectance spectrum of visible light, thus giving each code element 74 an identifying color. In some embodiments, coding portion 80 may be attached near only one of edges 90, 92. In these embodiments, the coding portion may partially or completely define or delineate only one side of the surface's perimeter.

Particles 70 may be manufactured using structural components that are blocks, sheets, or fibers, among others, of clear, colored, or otherwise modified glass and/or plastic, among others. The structural elements may be arranged in a bundle to form an assembly of coding and noncoding portions within the particles. Specifically, a separate structural component may be used to provide each of the code elements and the central noncoding portion. After and/or during fusion of the assembly, for example, by applying heat and/or pressure, the assembly may be stretched or drawn into a fiber. During the drawing process, the fused assembly may substantially maintain its cross-sectional aspect ratio. The resulting fiber may be cut to any desired length to form coded particles. Exemplary lengths (and thus particle thickness) are about 10-500 μm, 20-300 μm, or about 50-250 μm. Exemplary cross-sectional widths of the drawn fiber (and thus length and/or width of the particles) are about 10 μm to 2 mm, 50 μm to 1 mm, or about 100-750 μm.

1.7 Particles with Recesses and/or Magnetic Portions

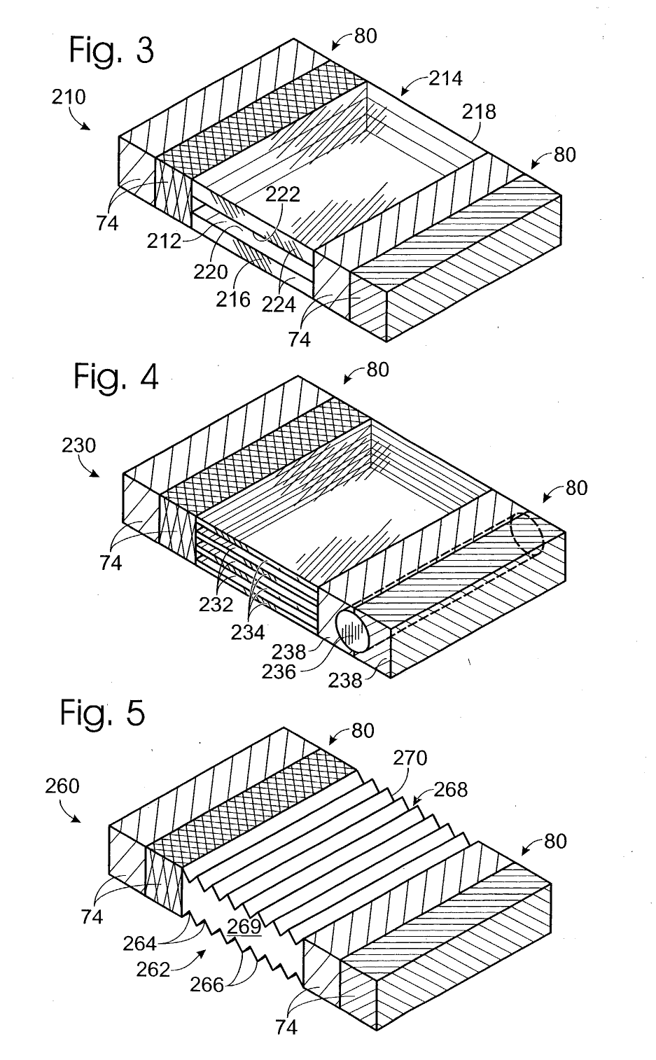

This section describes particles with recesses and/or magnetic portions, in accordance with aspects of the invention; see FIGS. 3 and 4. Methods for making such particles are also described.

FIG. 3 shows an embodiment of a particle 210 with a single recess 212. Particle 210 is structurally similar to particle 70 of FIG. 1, including coding regions 80 defined by code elements 74, which flank a centrally disposed noncoding portion 214. Recess 212 is included in noncoding portion 214 and, in this case, is a through-hole that extends between opposing sides 216, 218 of the particle. The recess defines interior association surfaces 220, 222 provided by walls 224 of noncoding portion 214. Here, the interior association surfaces are at least substantially parallel to exterior upper and lower association surfaces formed by walls 224.

FIG. 4 shows an embodiment of a particle 230 with plural recesses 232. As in particle 210 of FIG. 3, each of the plural recesses defines interior association surfaces that are at least substantially parallel to the upper and low exterior association surfaces of the particle. Each recess is bounded above and below by walls 234. Particle 230 also includes magnetic portion 236 in the form of a ferromagnetic or paramagnetic structure, in this case a cylinder, embedded between two code elements 238. Although particle 230 includes three recesses and a magnetic portion, in alternative embodiments this particle may be formed with zero, one, two, or greater than three recesses, and/or without the magnetic portion.

Particles 210 and 230 may be manufactured using similar methods. Blocks or fibers of glass are arranged and fused to form the general arrangement of coding and noncoding portions within the particles. Specifically, a separate component may be used for each of the code elements 74, each of walls 224 or 234, and the recess(es) 212 or 232. After and/or during fusion of the components, for example, by heating, the assembly is drawn into a fiber, during which magnetic portion 236, such as a wire, may be inserted and embedded into the assembly. After drawing the assembly to the desired size, the resulting fiber may be cut to any desired length. To allow formation of the recess, the assembly may be formed with a selectively removable material in the position of the future recess(es). In this case, an acid-sensitive glass is used at each recess position, whereas acid-resistant glass may be used to form the other structures of the particle. Acid treatment of the particle etches the acid-sensitive glass and removes it from the particle to create a recess.

1.8 Particles with Grooves/Ridges

This section describes particles with surface relief defined by grooves and/or ridges, in accordance with aspects of the invention; see FIGS. 5-11. Methods for making such particles are also described.

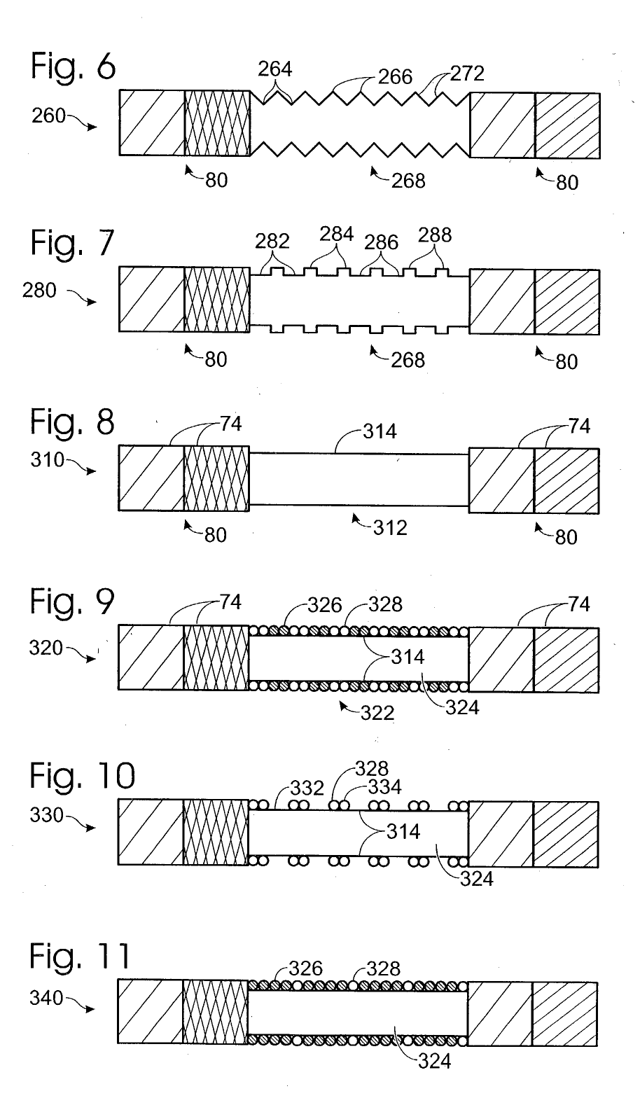

FIGS. 5 and 6 show perspective and side elevation views, respectively, of a fused-fiber particle 260 that is related to coded particle 70 of FIG. 1. Particle 260 includes a set 262 of grooves 264 and ridges 266 in noncoding portion 268. The grooves/ridges may be formed integrally with noncoding portion 268, with the grooves and ridges all being defined by a single component of particle 260. In this embodiment, the grooves and ridges extend to opposing sides 269, 270 of particle 260 and are generally linear and parallel. Furthermore, as shown in FIG. 6, grooves 264 and ridges 266 are defined by angled surfaces 272 that extend obliquely relative to the planes defined by opposing surfaces of particle 260.

FIG. 7 shows another embodiment of a particle 280 having grooves 282 and ridges 284 in noncoding portion 268. Grooves 282 and ridges 284 may be formed integrally with noncoding portion 268 from a single structural component of the particle, as in particle 280. However, particle 280 has both grooves 282 and ridges 284 defining surfaces 286, 288, respectively, which are generally parallel to the adjacent surfaces of coding regions 80.

The ridges and grooves of particles 260 and 280 may be formed by cutting grooves on one or both sides of noncoding portion 268. Although the grooves may be cut after the fused fiber has been drawn to its final cross-sectional size, the grooves may be cut more advantageously before the noncoding portion is drawn to its final size, that is, before or after the noncoding component(s) is fused to coding components of the particle. The grooves may be cut parallel to the axis along which the fused fiber is drawn, and the drawn fiber may be cut orthogonal to this axis.

FIG. 8 shows a particle 310 that includes a noncoding portion 312 that is offset. Particle may be formed of fused components, as for particle 260. However, the structural component that provides noncoding component 312 may be thinner than the components that provide coding elements 74. Accordingly, noncoding portion 312 may be fused to the other structural components so that surface 314 of the noncoding portion is not flush with the coding elements on one or both sides of the particle, thereby offsetting surface 314 in a recessed position.

FIGS. 9-11 show coded particles having surface relief defined by acid-sensitive and acid-resistant glass fibers that are attached to the particles. FIG. 9 shows a sectional view of a particle intermediate 320 formed from a fused bundle of structural components. Noncoding portion 322 may include noncoding component 324, which may correspond in size to noncoding portion 312 of particle 310 in FIG. 8. Accordingly, noncoding component 324 may be thinner than code elements 74. Recessed surfaces 314 defined by noncoding component 324 are attached to differentially sensitive fibers 326, 328, which may be disposed generally flush with code elements 74. Fibers 326, 328 may have any suitable diameter and cross-sectional shape, with exemplary fibers having a diameter of one or five microns. Sensitive fibers 326 are removable selectively with acid, whereas resistant fibers 328 are resistant to acid.

FIG. 10 shows a particle 330 produced from particle intermediate 320 of FIG. 9 by selective removal of sensitive fibers 326. Grooves 332 are formed at positions from which sensitive fibers 326 are removed. Ridges 334 are formed at adjacent positions at which resistant fibers 328 remain. Accordingly, the diameter of resistant fibers 328 may determine the ridge height, and the ratio and positioning of the two fiber types may determine the ridge and groove widths. Any suitable fiber ratio and size(s) may be used. For example, FIG. 11 shows particle intermediate 340, which is formed with a different ratio of sensitive fibers 326 and resistant fiber 328. Here, ridge-defining fibers 328 are outnumbered by removable fibers 326 by 4:1. Accordingly, removal of fibers 326 provides wider grooves and narrower ridges than in particle 330.

Particles having surface relief defined by selective removal of differentially sensitive fibers may be constructed using methods similar to those outlined above for other fused-fiber particles. The differentially sensitive fibers 326, 328 may be attached by fusion at the same time as fusion of code elements 74 to noncoding component 324, or such fibers may be attached to component 324 before or after component 324 is fused with code elements 74. Attachment to component 324 may be by heating, with light, through the use of an adhesive, and/or the like. Sensitive fibers 326 may be selectively removed from the particle intermediate at any suitable time during particle manufacture, including before or after drawing the fiber assembly (or progenitor structure) into its final aspect ratio, or after cutting the drawn fiber assembly into particle-sized units.

Grooves may facilitate exposure of a sample to a reagent when the sample is at least partially disposed between a surface of a particle and a flat substrate surface on which the particle is supported. Grooves also may facilitate the transfer and handling of the particle by disrupting long-range juxtapositions of the flat surface of a support and that of the particle (loss of surface tension binding). Grooves may define open-ended compartments in cooperation with the substrate surface. The sample may be at least partially contained in the compartment. Because the compartments are open-ended, reagent can access the compartment to contact the sample. Accordingly, such grooves may allow samples, particularly cells, to be analyzed on two opposing (generally upward-and downward-facing) surfaces of a particle. Further aspects of assays in which opposing surfaces of a particle may be used for sample analysis are included in the patent applications identified above under Cross-References and incorporated herein by reference, particularly U.S. Provisional Patent Application Serial No. 60/413,675.

1.9 Analysis of Magnetically Sorted Samples

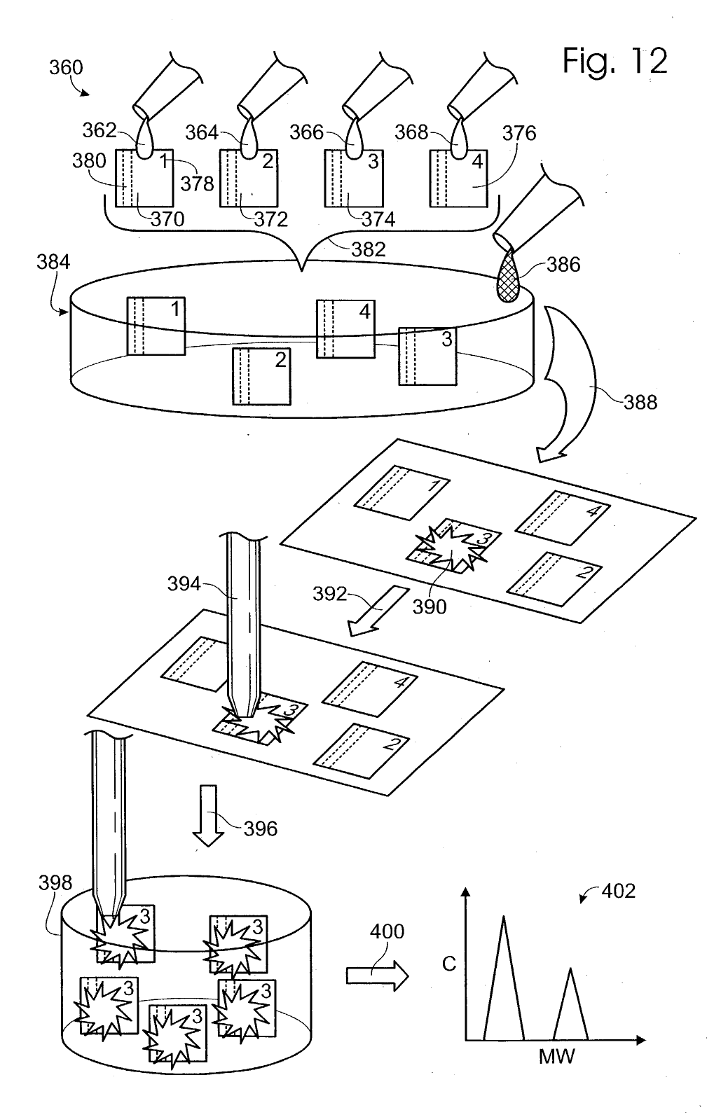

FIG. 12 shows a method 360 of using coded particles with magnetic portions to purify and analyze bound components, such as proteins, from a cell extract. As shown at the top of FIG. 12, proteins 362, 364, 366, 368 are associated with distinct classes of coded particles 370, 372, 374, 376, respectively, having distinct codes 378. Each of the particles also includes a magnetic portion 380 embedded in the particle. The resulting protein-particle assemblies may be combined, shown at 382, to form a nonpositional array 384 of protein probes. A cell or tissue extract 386 then may be combined with array 384 to allow specific components in extract 386 to bind to the particles. As shown at step 388, the protein-particle assemblies then may be measured to identify a positive signal 390 produced by bound extract components.

Individual particles that show a positive signal may be removed and further analyzed. As shown at step 392, a magnetic element 394 may be manually or automatically positioned near a particle with a positive signal (code “3”) to attract the particle to the magnetic element. As shown at step 396, particles that exhibit a positive signal and share a common code may be combined in a tube 398. The bound component from extract 386 then may be eluted from the particle, shown at step 400, and analyzed further. In this case, the eluted component, which represents a single species, is analyzed by mass spectrometry to determine structural features of the single species, as shown at 402.

In other embodiments, magnetic particles may be used to purify whole cells, tissues, phages, viruses, organelles, proteins, nucleic acids, carbohydrates, hormones, ligands, and/or chemical compounds, among others.

1.10 Optically Contrasting Regions to Locate the Assay Portion and/or Particle Perimeters

Juxtaposition of one or more optically contrasting regions to the assay portion may enable deduction of the positions of edges, sides, perimeter regions, and/or boundaries of the particle and assay portion, based on the identifiable positions of the contrasting regions. Some or all of the optically contrasting regions also may contribute to defining the code, so that optical recognition of the coding portion alone may partially or completely define the structure of the particle. Accordingly, once the coding portions of particles have been identified during image analysis, predicting where the associated assays portions are located may be much easier.

For example, consider a colorless, transparent square particle with an optically detectable circular coding region that is randomly located on the particle. If a group of these particles are bunched closely together, and in some cases overlapping, the circular coding regions may be easy to identify optically. However, locating and assigning an assay portion for each coding region may be very difficult because the perimeters of the particles are difficult to detect optically. The difficulty of detection may be compounded if the assay portion is difficult to identify optically, for example, because it lacks a detectable sample/reagent and/or assay result.

In some embodiments, the coded particles provided herein may reduce problems related to locating and assigning an assay portion for each particle. For example, when spaced coding regions flank an assay portion in a particle, the portion between the coding regions may be assigned readily as an assay portion. By providing a substantially constant spacing between spaced coding regions of a particle, particles and thus assay portions may be identified with greater reliability and thus confidence. More generally, if the relative placement of one, or more preferably, plural optically detectable (or contrasting) regions are known within a particle and relative to an assay portion, the particle perimeter and the assay portion may be deduced. Accordingly, judiciously shaped and/or located coding regions or other optically detectable (or contrasting) regions within a particle, may provide landmarks or reference points from which the particle perimeter and assay portion may be located.

### Example 2

### Coded-Particle Embodiments

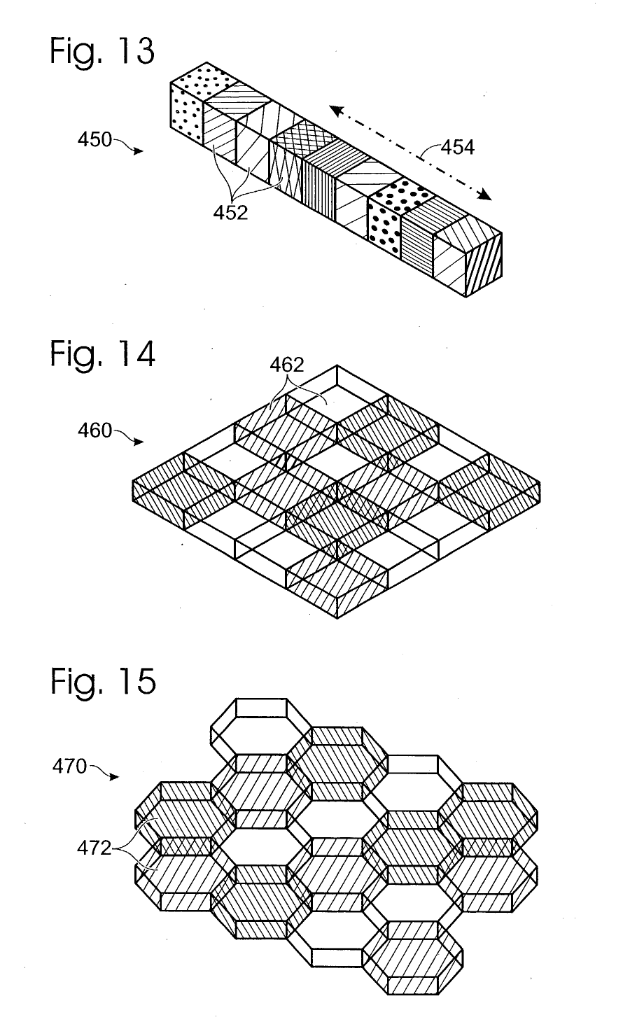

This example describes embodiments of coded particles for use in multiplexed assays, and methods for making the coded particles; see FIGS. 13-27.

FIG. 13 shows a coded particle 450 formed as an elongate parallelepiped. Particle 450 includes plural code elements 452, each having one of plural optical properties, in this case one of plural colors. Code elements 452 are arrayed generally parallel to long axis 454 of particle 450, so that the relative positions and colors of elements 452 along the axis define the code.

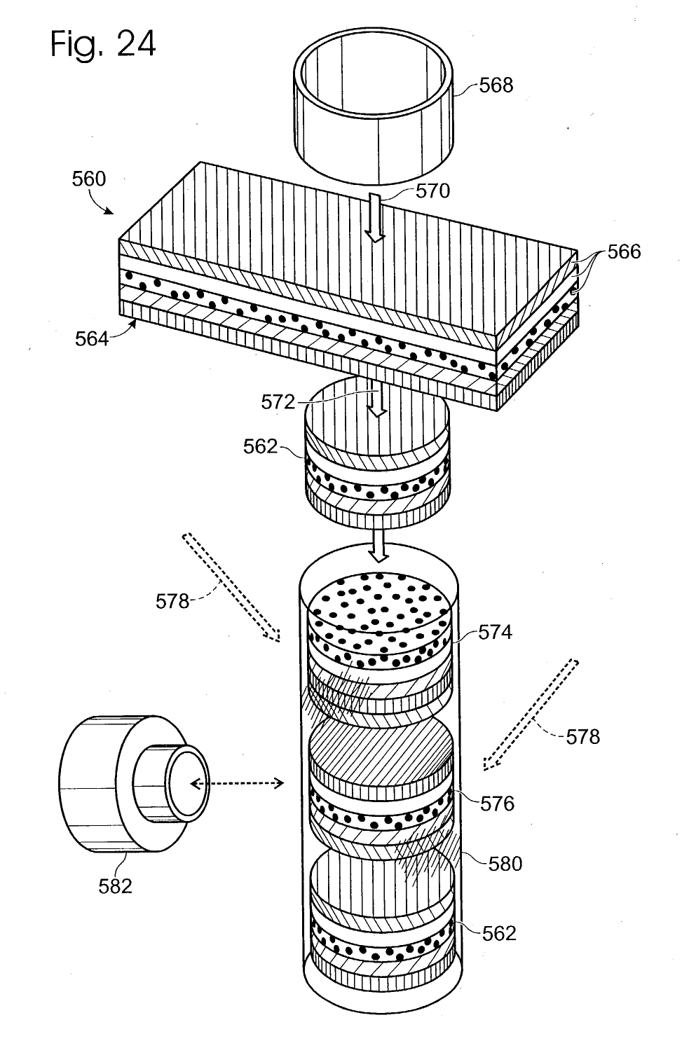

Particle 450 may be formed by any suitable method. For example, particle 450 may be formed from a fused fiber assembly, by cutting the assembly orthogonal to the long axis of the assembly. In this case, each fiber forms one of code elements 452. Alternatively, particle 450 may be formed from an assembly formed by fusing generally planar sheets of material (see FIG. 24), with each sheet defining one of the code elements. The sheet assembly may be cut along planes that are orthogonal to one another and to the plane defined by the sheet to release particle 450.

FIG. 14 shows coded particle 460 formed as a flat or generally planar parallelepiped having a two-dimensional array of code elements 462. Each code element may be square or rectangular in cross section so that the joined code elements define a square or rectangular upper and lower surface of the particle. In addition, each code element may have one of plural optical properties, so that the optical property of each code element and its position within the two-dimensional array define the code. Particle 460 may include an orientation feature (not shown) to define the starting point for reading the code (see Section I). Particle 460 may be formed as a fused fiber bundle, with each fiber defining one of code elements 462. The fiber bundle may be cut orthogonal to the long axis of the bundle (and thus orthogonal to the two-dimensional coding array) to produce individual particles 460.

FIG. 15 shows another embodiment of a generally planar or flat coded particle 470. Each code element 472 in particle 470 may have a detectable color. In addition, each code element may have a hexagonal cross section to form a honey-comb pattern. As a result, the edges of particle 470 may substantially lack points or sharp or rough edges that may contact and damage cells associated with other particles in a mixture. Particle 470 may be formed by cutting a fused bundle of fibers, as described above for particle 460. Shapes other than hexagons may be used to create compact arrays. For example, disks, polygons, cubes, triangles, octagons, or the like, may be used.

The structure of a particle may serve a number of purposes. For example, the geometry of a particle may serve as a coding indicium. Accordingly, particles may be distinguished by their appearance or by physical differences caused by their shape. The shape of a particle also may affect the particle's hydrodynamic character in a way that distinguishes each class of particle from one another. Shape may also play a role in how a particle displays itself (see Section VI above).

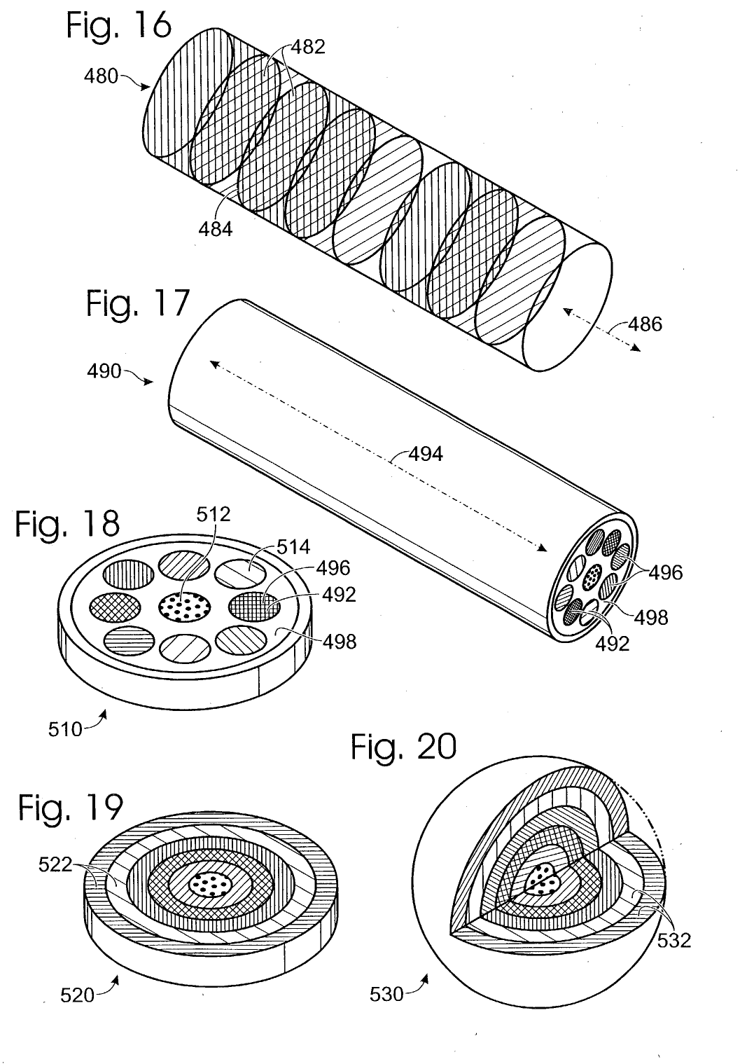

FIG. 16 shows an embodiment of a coded particle 480 based on a circular geometry. Particle 480 may be formed as code-element cylinders 482 fused at abutting flat surfaces 484. Thus, code elements 482 are arrayed generally parallel to the cylinder axis 486 of particle 480. This element array may be detected in a tube reader. More particularly, cylindrical particle 480 may self-orient as the particle enters a tube reader, so that the code element array, defined by code elements 482, is parallel to a detector of the tube reader.

FIG. 17 shows an alternative embodiment of a cylindrical coded particle 490. Particle 490 includes code elements 492 extending generally parallel to cylinder axis 494, and thus arrayed generally orthogonal to axis 494. Accordingly, the code may be viewed from an end of the cylinder, rather than from the side, as in FIG. 16. Particle 490 may be formed of fused fibers 496, each of which may define one of code elements 492, for example, by having one of two or more colors. Each fiber may be surrounded by a cladding 498. The cladding may be configured to limit optical interference between fibers during particle measurement.

Particle 490 is similar to a portion of a detection system disclosed by Chee, WO 99/67641, filed as U.S. patent application Ser. No. 09/189,543, which is incorporated herein by reference. In Chee, optical fibers having an outer cladding surrounding an inner core, where the inner core has been selectively etched back to produce cavities, thereby forming a particle holder.

FIG. 18 shows a coded particle 510 that is a disc embodiment of particle 490 (FIG. 17). Particle 510 may be self-orienting so that opposing particle surfaces are parallel to a substrate upon which particle 510 is manipulated or detected. Particle 510 may be produced by bundling fibers 496, and, optionally, cladding 498, and then stretching the bundle. The fibers may be attached to one another before, during, and/or after the stretching process. Individual particles 510 may be cut or sheared from the bundle. Fibers 496 may provide a paramagnetic core 512 for alignment and a position marker 514 to define a starting point for reading the code. The flat shape of particle 510 may provide improved optical access to the code to facilitate code determination.

FIG. 19 shows another cylindrical particle 520 that is defined by concentric rings of code elements 522. Code elements 522 define a linear code that may be read from the perimeter to the center or vice versa. Particle 520 may be a disk that self-orients to lie flat on a flat surface, as shown here, or particle 520 may be elongate, similar to particle 480 of FIG. 16. Particle 520 may be formed by stretching a parent cylinder formed of concentric rings and then cutting the stretched parent into individual particles.

FIG. 20 shows an embodiment of a spherical particle 530. Particle 530 may include concentric spherical layers of code elements 532, as shown in this cut-away view. In addition to, or alternative to a spatial color code, spherical particles or beads may be discernable by size, density, granularity, refractive index, or may contain yet another particle or particles that are further discernable. Beads may contain sub-populations of other, smaller beads distinguishable by color or other optical or physical features. Beads may be produced by a variety of methods including ultrasonic fluidic drop formation. Such methods may produce exceedingly uniform bead diameters and spherical shapes. Drop size may be highly controllable so that preparation of a library of different sized particles is possible. Beads also may be formed in a non-uniform manner, and then later sized by passing through a descending series of mesh screens. Polymer solutions used to form beads may themselves contain beads or particles, or combinations of each, smaller than the to-be-formed bead diameter. Examples of beads can be found in Bang's bead catalog, Flow Cytometry Standards catalog, and Molecular Probes catalog, each of which is incorporated herein by reference.

The invention further provides compositions where the particle coding element is a piece of a flat ribbon made of parallel glass fibers, and each fiber has one of at least two different colors, refractive indices or other optical properties. The invention further provides a method of fabricating particle codes made from fiber optic components, such as faceplates, windows, or image conduits as described in Hecht, “Understanding Fiber Optics”, 3 edition, 1998, Prentice Hall, incorporated herein by reference. In some embodiments, individual fibers may be in the range of from 1-500 or 3-100 μm. Optical fibers may be fused together to form structures consisting of a multitude of fibers in a variety of geometries. In manufacturing, starting with pre-forms, fiber assemblies may be drawn under heat and pressure such that they are parallel to each other; they retain shape and relative dimensions when drawn to a smaller size. Fibers may be made of transparent or colored glass or plastic. In some embodiments square fibers of transparent or colored glass or plastic are assembled in a flat ribbon pre-form. The order of differently colored fibers defines the code. The number of fibers depends on the desired number of classes of codes to be produced and the number of available colors. For example, with just two colors, 16 fibers may encode 64-thousand classes. The assembly then may be drawn to the size of approximately 10 μm to 4 mm across the ribbon and cut into segments having a thickness of about 1 μm to 4 mm. Cutting may be done individually by a laser, or after ribbons of the same class have been assembled in a bunch by a saw. A preferred use of particle area can be achieved with a 2-dimensional fiber, cut in 10-20 μm slices as in FIGS. 14 and 15. The assembly may be rectangular to have only two possible starting reading points, like in one-dimensional ribbons or multilayer particles (assuming that reading always starts from a corner along the longer side). One approach to uniquely identify a class is to use not all possible codes, but treat the codes that are reversals of each other as the same. This results in a little over a half of all codes usable. In some embodiments, particles may have the same number of code elements, but less than all code combinations may be used, to allow for code redundancy and error correction.

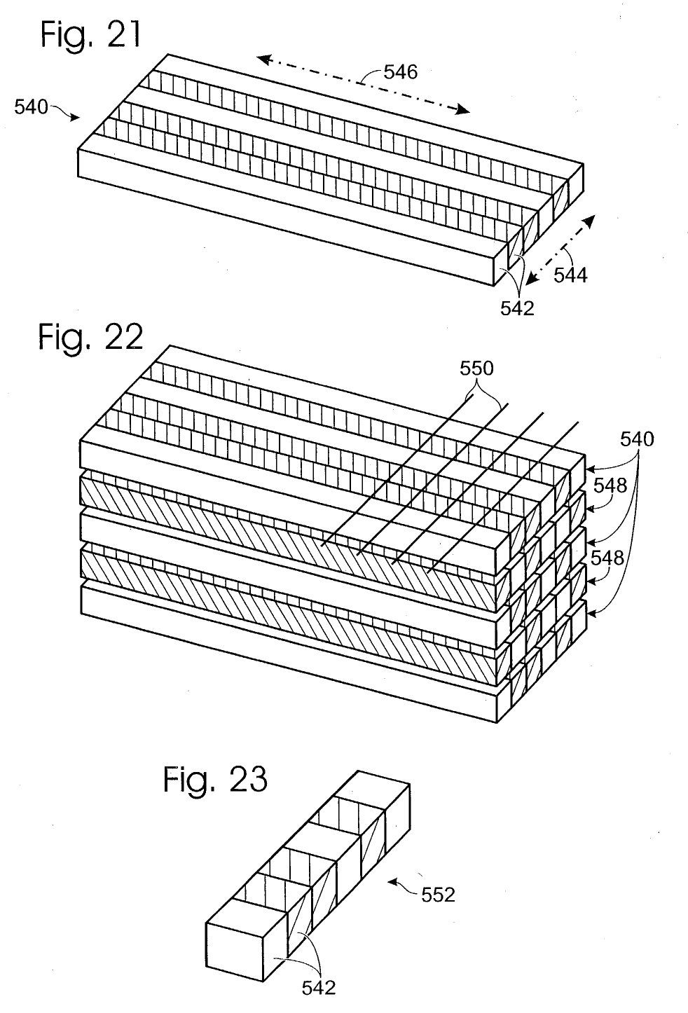

FIGS. 21-23 show formation of coded particles from coded ribbons that act as particle progenitor structures. FIG. 21 shows a coded ribbon 540 having code elements 542. Here, each code element is defined by a fiber that has only one of two optical properties to define a binary spatial code. However, in other embodiments, each code element may have one of any number of optical properties (or combinations of properties). Code elements (or fibers) define a coding axis 544 and a perpendicular element axis 546. The code elements extend parallel to element axis 546. The code element fibers may be attached to one another by bonding, fusing, heat fusion, gluing, or encasement by a sheath, such that the cross-sectional arrangement of the fibers is fixed. FIG. 22 shows plural coded ribbons 540 stacked together with distinct coded ribbons 548. The ribbons may be unattached to one another, or in other embodiments, the ribbons may be aligned and joined to form a two-dimensional fiber bundle having a two-dimensional coding array. In either case, ribbons 540, 548 or the two-dimensional fiber bundle (not shown) may be cut along cutting planes 550 to provide individual coded particles, such as particle 552 of FIG. 23.

Coded particles may be formed from a layered “sandwich” code. Such layered sandwiches may be formed by bonding film layers together to form a pattern in cross section. Like fibers or strands, film layers may differ from one another by chemical, optical, or electrical properties, among others. Chemical differences may include differential reactivity or isotopic differences, among others. For example, see U.S. Pat. No. 5,760,394, incorporated herein by reference. Indicia may also include radioisotopic differences and resistance to chemical attack. Optical differences may include colorimetric, reflective, granularity, polarization, and optical index. Electrical differences may include dielectric properties, where the sandwich yields a particular capacitance as a result of serially forming a capacitor sandwich, or the difference may be in resistance where each layer has a unique resistive value that can be combined to form a total and distinct resistance.

Combination approaches may include a layer sandwich punched out into distinguishable shapes. Differing coding structures may also be produced by extrusion, molding, spray formation, electrospray deposition, vapor deposition, machining, punching, or may be naturally diverse, for example, particular species of diatoms. Structure differences may also occur at the atomic or polymeric level, for example, as with “bucky balls.”

In use, a composition containing up M^{N} different coded particles, each formed with a different surface-attached compound, for example, oligonucleotide, oligopeptide, or small organic compound, is reacted with a target, for example, receptor molecule, under conditions which lead to binding of the target to beads carrying compounds that bind specifically to the target. Preferably the target molecules are labeled, e.g., with a colored or fluorescent reporter. The particles are then fed into a capillary flow tube, past a detector, where the particles are first scanned for the presence of target binding. For those particles that have bound target, a second scanning device then “decodes” the pattern of colors of the device, to identify the compound on the particle according to its particle code. It will be appreciated that other types of particles, for example, cylindrical or rod-shaped particles, that can be oriented in a capillary flow tube, and which can be encoded in a top-to-bottom fashion, e.g., with different layers having individually identifiable indicia, can be employed in the method. Thus, cylindrical particles having layers of different fluorescent labels can be “decoded” in the same fashion. Alternatively, the particles may have a magnetic layer or component that allows for magnetic separation of said particles.

FIG. 24 shows a method 560 for forming coded cylindrical particles 562 from a layered-sandwich assembly 564. Assembly 564 includes joined layers 566, each having one of two or more optical properties. Punch 568 may excise particles 562 as cylinders, as shown at 570 and 572, or, as described above, two or more punches with distinct shapes may be used to define particles in which the particle shape defines at least a portion of the code.

Distinct sandwich assemblies (not shown) may be used to form other classes of coded particles 574, 576. The coded particles may be associated with distinct samples/reagents and combined, as indicated at 578, to perform a multiplexed experiment. An experimental result and the particle codes may be read by flowing the particle in a capillary tube 580 past a detector 582, which may image code elements sequentially or simultaneously to determine the code.

The invention further provides for encoded particle “chips” containing an embedded code. The particles may be of the same overall size and shape, but may include code elements that are optically distinguishable. The manufacture of such microchips, containing optically identifiable marks, is a standard practice in the microelectronic industry. See generally, “Semiconductor Materials and Process Technology Handbook”, G. E. McGuire—ed., Noyes Publications, Park Ridge, N.J., U.S.A., 1998, incorporated herein by reference.

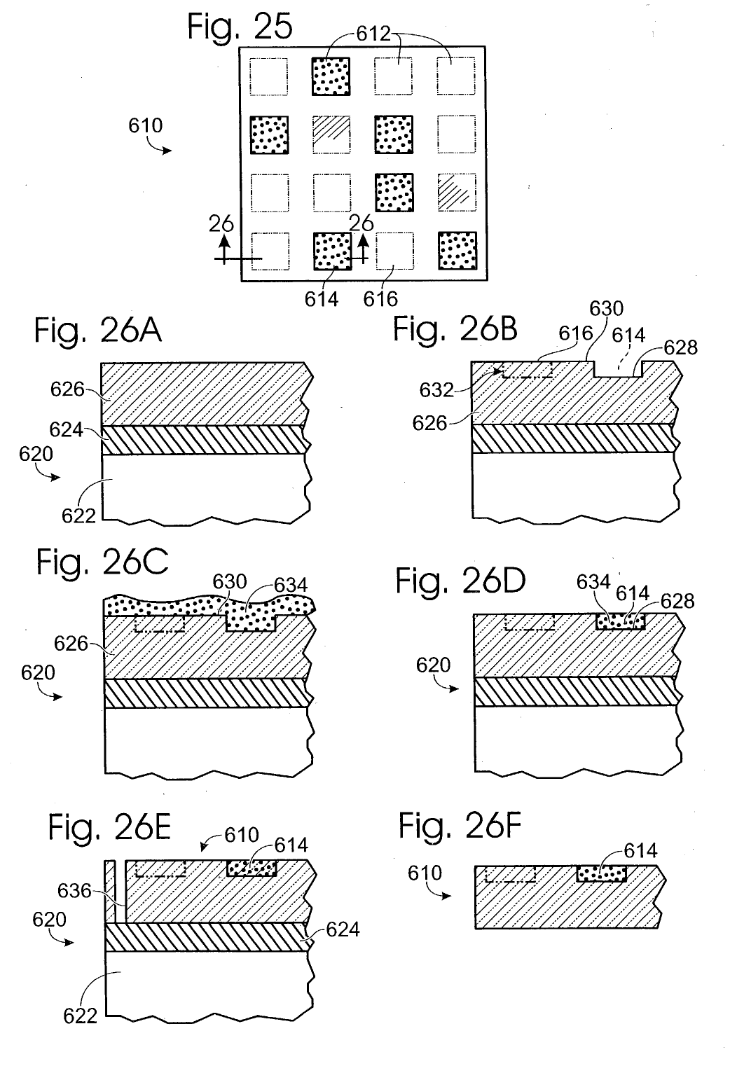

FIG. 25 shows an exemplary coded microchip 610 having a plurality of binary code positions 612, or sixteen in this embodiment. Each code position 612 may have one of two optical properties or identification features, or may have or lack the feature, as shown at 614 and 616, respectively, to provide 2¹⁶ or 65,536 particle codes. Identification features may differ in any suitable optical property, for example, transmission or reflection, among others. Each identification feature may have any suitable size, for example, a size of about 1-100, or about 2-4 square μm.

FIG. 26 provides an exemplary method for fabricating the coded microchip of FIG. 25.

FIG. 26A shows a layered assembly 620 from which microchip 610 may be formed. Assembly 620 includes a silicon substrate 622 upon which other layers are formed. Substrate 622 may have an exemplary thickness of about 2 μm. A layer 624 of PETEOS (Plasma Enhanced Tetra-Ethyl-Ortho-Silicate) may be formed on silicon substrate 622 and may have an exemplary thickness of about 0.5 μm. PETEOS layer 624 may allow the finished microchip to be removed from the wafer substrate (see FIG. 26F below). Polysilicon layer 626, with an exemplary thickness of about 2 μm, may be formed on substrate 622, above PETEOS layer 624. Polysilicon layer 626 may provide the bulk of the structural material for microchip 610.

FIG. 26B shows polysilicon layer 626 after patterning and pattern-based etching. Patterning may be performed using photolithography with a mask that defines positions of identification features 614. Plasma etching of the patterned polysilicon layer 626 creates a recess 628, in this case having a depth of about 0.5 μm in surface 630 of layer 626. By contrast, unetched code element 616 includes no recess, shown in dashed outline at 632.

FIG. 26C shows assembly 620 after deposition of identification-feature film 634 on surface 630 of polysilicon layer 626. Identification-feature film 634 provides optical contrast to polysilicon layer 626. Identification-feature film 634 may be any optically contrasting material. Exemplary materials for film 634 include silicon nitride or a metal film (aluminum or tungsten, for example) for analysis of transmitted or reflected light.

FIG. 26D shows assembly 620 after removal of excess identification-feature film 634 disposed above surface 630. Film 634 in recess 628 is selectively retained to form code element 614. In the case of a metal film, metal may be removed by chemical mechanical polishing (CMP), leaving metal only in recess 628.

FIGS. 26E and 26F show final delineation and release, respectively, of microchip 610. FIG. 26E shows assembly 620 after photolithography and etching to define a border 636 of microchip 610 where polysilicon layer has been removed down to PETEOS layer 624. FIG. 26F shows microchip 610 after wet etching in dilute (50:1) hydrofluoric (HF) acid to remove PETEOS layer 624 and thus separate the microchip from substrate 622.

Coded particles may be formed by multiplayer soft lithography (MSL). MSL is described, for example, in Marc A. Unger, Hou-Pu Chou, Todd Thorsen, Axel Scherer, Stephen R. Quake, “Monolithic Microfabricated Valves and Pumps by Multilayer Soft Lithography” Science v. 288, pp. 113-116, Apr. 7, 2000, which is incorporated herein by reference. The method described by Unger et al. may be modified to use resins with distinct optical properties as layers. The distinct optical properties may be defined by dyes included in the resins. After a multilayered coded sheet is formed, the sheet may be “cut” in parallel into squares or cylinders by exposing it to eximer laser light. Such a cutting service is provided by Resonetics, Inc. www.resonetics.com. Other references that teach microfabrication and fiber optics are incorporated herein by reference: Fundamentals of Microfabrication, by Marc Madou, CRC Press (1997); Fiber Optic Networks, by Paul Green, Prentice-Hall (1993); and Non-linear Fiber Optics, by Govind Agrawal; Academic Press (1995). Microparticles can be made by an adaptation of soft lithography techniques described by Unger, et al. in Science volume 288, page 113-116, April 2000, where multiple layered substrates are etched to form microparticles having on at least one side a viewable coding region.

Another extension of the above method includes molding complementary patterned layers having distinct optical properties such as first and second colors. When such layers are fused together and cut, flat particles result that have a pattern of predominantly the first or the second color.

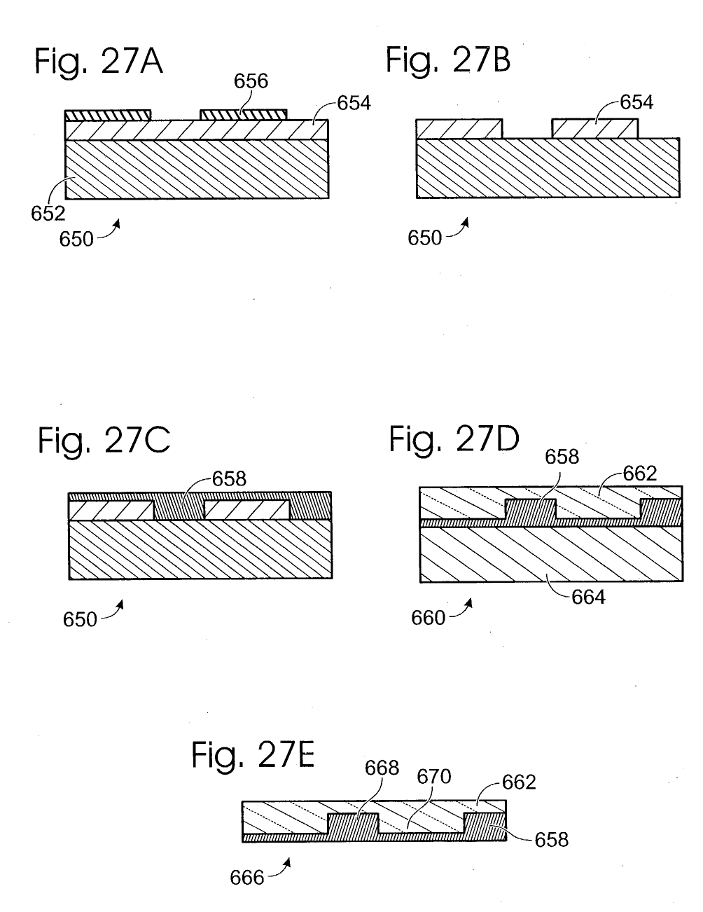

FIG. 27 shows structures produced during formation of a coded particle using multilayer soft lithography. FIG. 27A shows a mold assembly 650 having a substrate 652 upon which a layer of photoresist 654 has been formed. A mask 656 partially covers photoresist 654. FIG. 27B shows patterned removal of photoresist 654 after mask-directed light exposure and development, and removal of the mask. FIG. 27C shows an elastomer layer 658, which has been formed by spin-coating an elastomer, such as GE Silicones RTV 615, onto mold assembly 650. The elastomer may include an added optical contrasting agent. After application of the elastomer, the elastomer may be baked. FIG. 27D shows use of elastomer layer 658 in a new mold assembly 660 to form a complementary, but optically distinct, second elastomer layer 662. Elastomer layer 658 has been peeled from substrate 652, inverted, and placed on another substrate 664. Second elastomer layer 662 then may be spin-coated onto the mold defined by first elastomer layer 658, using the same elastomer but without addition of the optical contrasting agent. FIG. 27E shows completed multi-layer coded sheet 666 after baking and separation from substrate 664. Coded sheet 666 may be cut orthogonal to a coding axis of the sheet to form coded particles. Code elements may be defined by the shape, size, and position of thickened regions 668 and/or 670 of layers 658, 660, respectively.

### Example 3

### Film-based Coded Particles

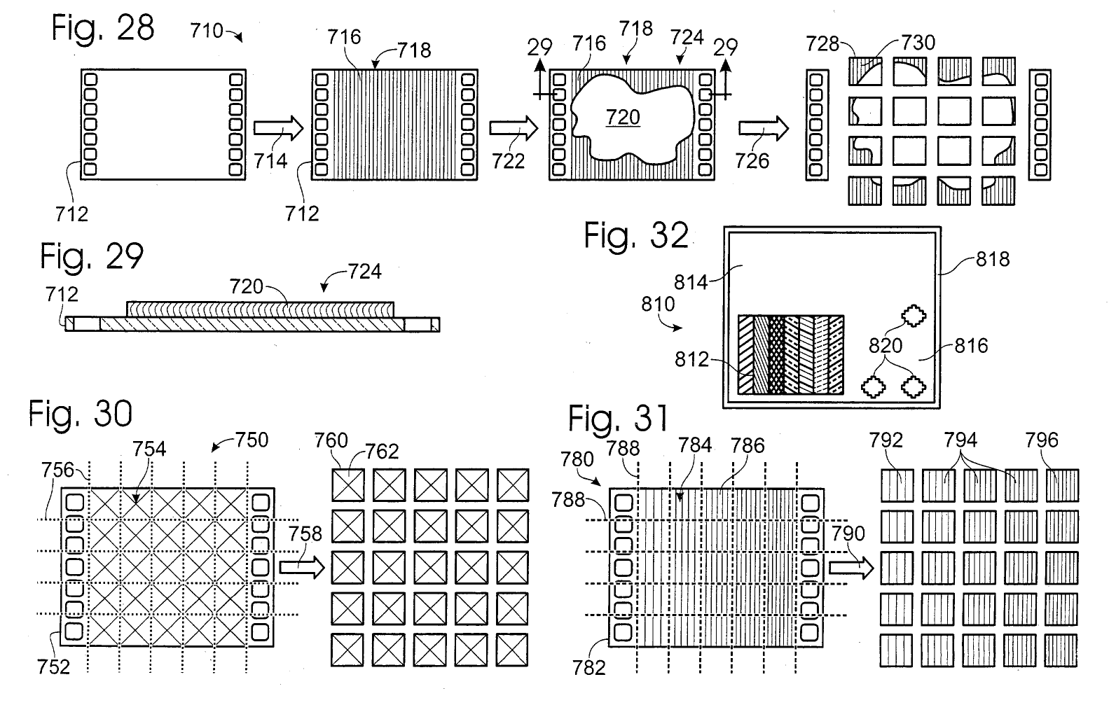

This example describes coded particles (or carriers) comprising photosensitive film for use in nonpositional and/or positional arrays; see FIGS. 28-32.

There are many methods for making coded particles. However, these methods may be complex and/or expensive to carry out, particularly if the particles are to have an extremely small dimension, for example, 400 microns or less. Thus, there is a need for a simple and inexpensive method for making coded particles.

The invention provides coded particles and methods for making coded particles for use in analysis of biological and/or other samples. The particles may be particularly useful for multiplexed analysis of biological samples.

FIG. 28 shows a method 710 of making coded particles in accordance with aspects of the invention. In method 710, a photosensitive film 712 is exposed, shown at 714, to a base image or pattern to form a replica or film image 716, for example, as a pattern on the film, thus coding the film. Coded film 718 then may be cut into a plurality of small coded particles having the same or different codes suitable for use as particles for biological samples, such as nucleic acids, polymers, proteins, cells, tissue slices, etc. Alternatively, a biological sample, such as tissue section 720, may be immobilized to coded film 718 prior to cutting, shown at 722, essentially creating an open-faced sandwich 724; see FIGS. 28 and 29. The film/sample sandwich 724 then may be partitioned, shown at 726, into a plurality of segments 728. In either case, each segment of film, or particle, to which the biological sample is immobilized includes an image or coding portion 730 of replica image 716. Thus, image portion 730 may act as an identifiable code, allowing the coded particles, and thus the associated biological samples, to be identified and tracked throughout analysis.

A base image generally comprises any image that may be photographed, projected onto, or otherwise reproduced on photosensitive film. The image may or may not include a reproducible pattern. Suitable images include, for example, easily identifiable patterns, such as stripes, grids, repeating shapes (such as spots), and the like. In some embodiments, the image includes a repeated pattern of stripes of different colors.

Film 712 generally comprises any thin sheet or strip of photosensitive material capable of recording and/or used to record a photographic image, including but not limited to cellulose derivatives and thermoplastic resins coated with a photosensitive emulsion and used to make photographic negatives or transparencies. Any suitable film may be used, including black and white or color film, depending on the nature of the code and the application. Commercial film may be suitable, although some commercial films are intrinsically fluorescent, which may interfere with reading the code, and/or the results of luminescence and/or colorimetric assays. In these cases, the code and/or assay may be read using a wavelength and/or other property separately detectable from the film fluorescence.

The film may be exposed to the base image using any suitable technique. For example, the film may be exposed to the base image by photographing the base image using a suitable detection device such as a camera. In brief, when a camera's shutter is open, the lens focuses light originating from the object in the field of view of the camera onto the film. Such light may arise from reflection, transmission, and/or emission from the object. Photosensitive chemicals in the film react to exposure to the light. When the film is developed, the reacted areas change properties, for example, changing colors and/or opacity, among others, such that the base image is recorded on the film as a film image, creating a negative. Color film typically makes use of three dyes corresponding to the three primary colors: blue, yellow, and red. More generally, the film may be exposed to the base image or pattern by directing light or other radiation directly onto the film, for example, using direct laser or CRT writing, with or without the use of any imaging optics.

The coded particles of the invention are particularly useful for multiplexed analysis, as stated above. Multiplexed analysis typically involves conducting experiments on a number of different samples from different sources pooled together. This multiplexed approach may save the researcher a significant amount of time and expense, and it allows for a better comparison of results from different sample sources. However, multiplexed analysis also may require a determination of which sample came from which source to interpret the results of the experiments. For this reason, it often is desirable to label samples from different sources with different identifiable markers or codes.

The simplest multiplexed analysis involves study of two different types of samples, for example, a first sample and a second sample that differ in kind and/or condition, among others. These samples may comprise different types of cells, tissues, etc., such as Swiss 3T3 and HeLa cells, or kidney and uterine tissues, among others. Alternatively, or in addition, these samples may comprise the same types of cells, tissues, etc., taken at different times and/or under different conditions, among others.

Multiplexed analysis of two types of samples may be used to conduct experiments on a particular tissue or other sample before and after treatment with a particular chemical to test the effects of that chemical on the tissue. In such experiments, a sample of the tissue is obtained prior to treatment (the pretreatment sample), and a sample of the tissue is obtained after treatment (the posttreatment sample). The pretreatment sample is labeled with a first code, and the posttreatment sample is labeled with a second, distinguishable code. To accomplish this, two different base images are photographed or otherwise reproduced on film to form two different replica or coded images. The pretreatment sample is immobilized to film displaying the first coded image, and the posttreatment sample is immobilized to film displaying the second coded image. Thereafter, the sandwiches are cut into particles or particles. The particles then may be combined and experiments may be conducted on all or a portion of the pooled particles. Detection of the pattern (or coded image portion) displayed by the particle will indicate whether a given particle supports tissue from the pretreatment or posttreatment sample.

More complex multiplexed analysis may involve study of three or more samples, for example, a first, second, and third sample that differ in kind and/or condition, among others.

In a multiplexed analysis, for the various samples to be distinguished after the samples have been pooled, the images recorded on the film must be distinguishable after the film (and generally the film image) has been partitioned into particles (and image portions). Thus, the images/image portions used to code the particles may be chosen to allow the user to distinguish between different samples.

The film image may be designed such that the image portion on each particle derived from a single frame of film will be identical when the film is cut into pieces of a given size. For example, method 750 of FIG. 30 shows a single frame of film 752 having a film image 754 that is a repeating pattern of the letter “X”. After cutting along lines 756 between each “X”, shown at 758, each particle 760 contains an “X” as image portion 762. Thus, all particles having an “X” as their identifying code may carry a portion of the same biological sample.

Alternatively, the film image may be designed such that the image portions on two or more particles derived from a single frame of film will be distinguishable from each other when the film is cut into pieces of a given size. In this case, the film image produces at least two distinct codes (and coded particles) when divided into image portions. In some embodiments, all particles derived from one or more frames of film may have different codes, making them particularly useful in combinatorial applications or in-situ synthesis. An example of a method 780 for forming distinct coded particles along one of two cutting dimensions is shown in FIG. 31. Here, a single frame of film 782 includes a film image 784 of vertical bands 786, and the vertical bands have a decreased width, from left to right, along the film. After cutting along lines 788, shown at 790, particles 792 from the far left-hand side of the film have four bands, particles 794 from columns progressively moving to the right have five, six, or seven bands, and particles 796 from the far right-hand side of the film have eight bands. Thus, when associated with a tissue section, as in method 710 of FIG. 28, a particle having four bands will be determined to carry a portion of the far left-hand side of the tissue, a particle having five to seven bands will be determined to carry a portion of the middle of the tissue, and a particle having eight bands will be determined to carry a portion of the far right-hand side of the tissue. In other embodiments, the particles may have the same number of bands but different codes as determined by properties of the bands, such as the size, position, color, and intensity of the bands. In yet other embodiments, the particles may have codes determined by properties of the particles other than bands.

The film image may be designed so that the code is positioned at any suitable location or locations on the particle, including the entire particle or a portion or portions thereof. A code positioned only at a portion of the particle effectively divides the particle into a coding portion and a noncoding portion. Assays such as cell assays then may be performed, if desired, only at noncoding portions, even if cells or samples are associated at both regions, to reduce any possible interference between the code and the assay. In these assays, the noncoding portions, or portions thereof, effectively constitute an assay or measuring region.

The film image also may include additional (i.e., noncoding) features, such as alignment marks that may be used independent of the code to align the image of the particle before interpreting the code. Suitable alignment marks include spots, crosses, and/or other shapes positioned at defined positions on the particle relative to the coding and/or noncoding portions.

The film can be cut into any number of shapes and any number of sizes, although the figures show film being cut into a 5×5 grid of squares. An individual film frame may be used as a particle without portioning the film. Typically, however, it is desirable for the particles to be smaller than an individual frame of film. Thus, the film may be cut into particles having a largest characteristic dimension between about 0.001 and 35 mm, between about 0.01 and 5 mm, or between about 0.1 and 1 mm in diameter, among others, depending on the properties of the particle and/or the application. Generally, smaller particles will be better prepared with thinner, finer grained film. For standard photographic film, the film is about 0.130 mm thick, so the largest dimension will be somewhat larger than this value.

The film and/or sandwiches may be portioned using any method capable of cutting or otherwise separating the film into portions, including, for example, mechanical means such as a sharp cutting edge or punch, manual means such as tearing, chemical means such as etching, and/or optical means such as laser cutting. The portioning may be facilitated using any suitable mechanism, including guidelines, perforations, and/or scoring. In some embodiments, the film may be precut into a plurality of portions that remain attached to one another and/or to a common surface by a dissolvable attachment substance, such as gelatin. Individual particles then may be created by dissolving the attachment surface, before or after immobilizing or in-situ synthesizing a sample, such as a biological sample on the precut film.

The particles may be portioned into separate coded particles before and/or after immobilization of the sample onto the particles. Thus, in some embodiments, film encoding a suitable pattern may be cut into a first set of pieces, a set of samples may be affixed to the pieces, and then the pieces and affixed samples may be cut further into a second set of pieces for analysis.

Methods of use for the coded particles are described elsewhere in this Detailed Description. Once the experiments are conducted, the origin of the samples immobilized on each particle can be determined by observing the pattern or image portion displayed by the particle. Typically, the particles are viewed by a microscope and/or with a film scanner, although more generally any suitable detection device may be used. Films may be scanned at any desired resolution, with the preferred resolutions limited by film grain size. Commonly, films are scanned with a resolution of about 6-10 microns per pixel.

The number of different codes available is determined by interplay among the size of the particle, the grain size of the particle, and the base image selected. Most common films are between 130 and 170 microns thick. At this thickness, for the particle to lie flat (to enable viewing or scanning), the particle should have a width of about 400 microns or more. The size and/or density of the coding features is determined by the grain size of the film. For example, if a coded image including a series of stripes, bands, or other features is chosen, it generally is desirable for each band to have a width of between about 4-5 pixels; thus, each stripe typically is about 25-50 microns in width. Consequently, each particle can have more than 10 bands. If color film is used, each band may be of a different color; as a result, the total number of possible codes is nearly unlimited. Use of specialty or custom-built films may reduce the grain size of the film and/or the thickness of the base and allow for even smaller particles.

3.1 Exemplary Method for Making a Color-Coded Particle

An exemplary embodiment of a method for making a color-coded particle is described here. A color stripe chart of a repeated pattern with four colored stripes is printed on an ink jet printer and photographed with a 35 mm camera. The film is developed and cut into approximately 0.5 mm squares. These squares are mounted in a slide frame and scanned with a film scanner at the resolution of about 6 microns per pixel. The color patterns are easily recognized by eye and computer, with the four-stripe pattern repeating about every 200 microns.

3.2 Alternative Color-Coded Particle

FIG. 32 is an image of an alternative color-coded particle 810 produced in accordance with the invention. The particle includes a coding portion 812, a noncoding portion 814 that is spatially distinct from portion 812, an alignment region 816, and a frame 818. The coding portion may be used for containing a code for identifying the particle or particle type. The noncoding portion may be used for conducting assays on cells, tissues or other samples affixed thereto. The alignment region may be used for aligning a detection system prior to reading and interpreting the code and/or analyzing the sample. The alignment region includes an asymmetric set of spots 820 positioned at predefined positions relative to one another, the coding portion, and the noncoding portion. The frame may be used to define the exterior of the particle, or the usable region of the particle, and may serve as a guideline for separating the particle from other particles during manufacture and/or subsequent use.

3.3 Alternative Color Film

This section describes methods for producing a color image on a film. These methods may be useful in providing more temperature- and/or solvent-resistant film-based coded particles from the film, for use in multiplexed assays. Standard technologies for producing color film provide a basis for the methods in this section and thus will be reviewed here.

Standard color film is generally formed of multiple layers. Three of the layers may be color layers, and at least one additional layer may be a filter layer that controls the color of light that enters some of the color layers. The color layers each contain silver halide grains and a distinct dye in a gelatin matrix, typically as a photosensitive emulsion. Each dye is generally colorless and soluble prior to reaction with developer. However, after reaction with developer, the dyes may become colored and immobilized to their respective color layers, for example, via long alkyl chains embedded in the gelatin matrix of each color layer.

Light initiates the sequence of chemical reactions that forms and immobilizes colored dyes in the color layers. Exposing film to light spatially activates silver halide grains in the appropriate color layers, based on the intensity and distribution of light of different colors. During the development process, the film is immersed in a developer that enters each color layer and interacts with the activated silver halide grains to form free radicals from the developer. The free radicals then couple locally with nearby dye. This coupling reaction converts the previously colorless dye to a colored dye and renders it insoluble in the gelatin matrix, essentially fixing the location of the dye. Also, during a subsequent process, the silver halide grains and the filtration functionality are removed from the film. Therefore, the final image, although initiated by light activation of the silver halides grains, contains no such grains itself, and instead is formed by immobilized dyes.

Despite the usefulness of standard film technology to produce coded particles, the technology may not be suitable to produce coded particles for some multiplexed analyses. Specifically, the gelatin matrix in each of the color layers may be unable to withstand the elevated temperatures and/or organic solvents used in many cell assay and hybridization protocols. Therefore, alternative methods may be needed that form spatially immobilized dyes in film, thereby allowing production of versatile coded particles for multiplexed assay systems.

This section provides new films that form images by light-mediated coupling of dyes to immobilized developer. The developer may be immobilized on exterior surfaces of and/or interior regions of the film. In turn, the exterior surfaces and/or interior regions of the film may be exposed to one or plural dyes and light; exposure to the plural dyes may be serial or in parallel. Light exposure may be localized and/or patterned, for example, by using a laser and/or photolithography. Localized and/or patterned light forms an image, typically a colored image, of immobilized dye or dyes coupled to the developer. Coupling may include covalent linkage and/or noncovalent interaction between the dye and developer. Furthermore, coupling also may convert the dye from a colorless to a colored form and/or immobilize the dye.

Coupling of developer to dye may be carried out by various mechanisms, generally without the need for silver halide grains. The function of the silver halide grains in standard color film may be substituted with a light-activated free-radical initiator, for example, riboflavin, to indirectly activate the developer with light. In some cases, strictly chemical free-radical initiators, such as hydrogen peroxide, may be used, to activate the developer. Alternatively, the developer may be directly light-activated, and a separate free-radical initiator may not be required. In this case, distinct light-activated developers may be used that are differentially sensitive to distinct colors of light. Furthermore, such light-activated developers may be specific in their ability to couple to dyes of distinct colors.

In some embodiments, a dye or dyes may couple to a developer presented on the exterior surface of a film. All or a portion of the film's exterior surface may present developer for interaction with dyes. The developer may be covalently linked with, or may noncovalently adhere to, the film after it has been formed. Alternatively, or in addition, the developer may be incorporated into the film during formation of the film.

A region or band of the exterior surface of the film may be colored as follows. The film is exposed to dye and a free radical initiator, for example, a solution of a red dye and riboflavin. Illumination of the band with UV light locally activates the riboflavin, which in turn locally activates the immobilized developer. As a result, the activated developer reacts with proximate dye. Thus, in this case, red dye is coupled to the immobilized developer, forming a red band on the surface of the film. Unreacted red dye then is washed away. Additional colors may be added to the exterior surface by repeating the procedure with other dyes. For example, the film may be exposed to a green dye and riboflavin, and then a different region of the film may be illuminated, producing an immobilized band of green dye on the film. Single dyes or combinations of dyes may be used for each band or other code element.

In other embodiments, one or more color chemistry materials, i.e., developer, dye, and/or free-radical initiator, may be directly incorporated into a film, such as a film formed from an epoxy resin. These color chemistry materials may be covalently attached to film precursors, such as monomers that polymerize to form the film. Alternatively, these materials may be trapped physically within the film during its formation. Furthermore, these materials may be introduced after film formation by swelling the film with a suitable solvent that includes one or more of the materials. As a result of one or more of these approaches, the color chemistry materials may be uniformly incorporated into the film. Various regions of the film may then be illuminated by light of various colors, producing colored regions in situ. For these embodiments, a dye and developer system may be used in which colorless dyes are specifically activated by a particular wavelength or wavelengths of light, and then become chromogenic, i.e., exhibit a color upon reacting with developer.

In any of the above embodiments, uncolored or specifically colored regions on the film may define a cutting path for an automated laser or other cutting device. However, more generally, the film may be portioned to form plural coded particles by any suitable mechanism.

3.4 Selected Embodiments

This section describes further aspects of the invention, as set forth in the following numbered paragraphs:

1. A method of producing a coded microparticle, comprising: exposing photosensitive film to a base image such that a replica of the image is recorded on the film; and cutting the film into a plurality of portions to produce a plurality of coded microparticles.

2. The method of paragraph 1 wherein the base image includes an optically distinctive pattern.

3. The method of paragraph 2 wherein the pattern is in black and white.

4. The method of paragraph 2 wherein the pattern includes a plurality of colors.

5. The method of paragraph 1 wherein the base image is obtained by printing.

6. The method of paragraph 1 wherein the step of exposing is performed by photographing the base image with a camera.

7. The method of paragraph 6 wherein the camera is a 35 mm camera.

8. The method of paragraph 1 wherein each portion has a largest characteristic dimension between about 0.1 mm and 5 mm.

9. The method of paragraph 1 wherein each portion has a largest characteristic dimension between about 0.1 mm and 2 mm.

10. The method of paragraph 1 wherein each portion has a largest characteristic dimension between about 0.1 mm and 1 mm.

11. A method for encoding a biological sample, comprising: exposing photosensitive film to a base image such that a replica of the image is recorded on the film; immobilizing a biological sample on the film; and cutting the film into a plurality of portions.

12. The method of paragraph 11, wherein the step of cutting is performed before the step of immobilizing.

13. The method of paragraph 11, wherein the step of immobilizing is performed before the step of cutting.

14. The method of paragraph 11, wherein each portion includes a part of the biological sample and a part of the coded image.

15. A method for encoding a biological sample, comprising: exposing photosensitive film to a base image such that a replica of the image is recorded on the film; cutting the film into a plurality of portions, wherein each portion includes a part of the coded image; and attaching a biological sample to the portions of the film.

16. A method for encoding a biological sample, comprising: exposing photosensitive film to a base image such that a replica of the image is recorded on the film; precutting the film into a plurality of portions while keeping them attached to a common surface by a dissolvable attachment substance; immobilizing or synthesizing in situ a biological sample on the precut film; and dissolving the attachment substance, thereby releasing the plurality of portions, wherein each portion includes a part of the biological sample and a part of the coded image.

17. A coded microparticle comprising a photosensitive film, the film having been exposed to a base image such that a replica of the image is recorded on the film.

18. An encoded biological sample comprising a biological sample immobilized to a photosensitive film, the film having been exposed to a base image such that a replica of the image is recorded on the film.

19. A composition of matter, comprising: a coded microparticle according to paragraph 17; and an encoded biological sample according to claim 18.

### Example 4

### Particles With Surface-Contour Codes

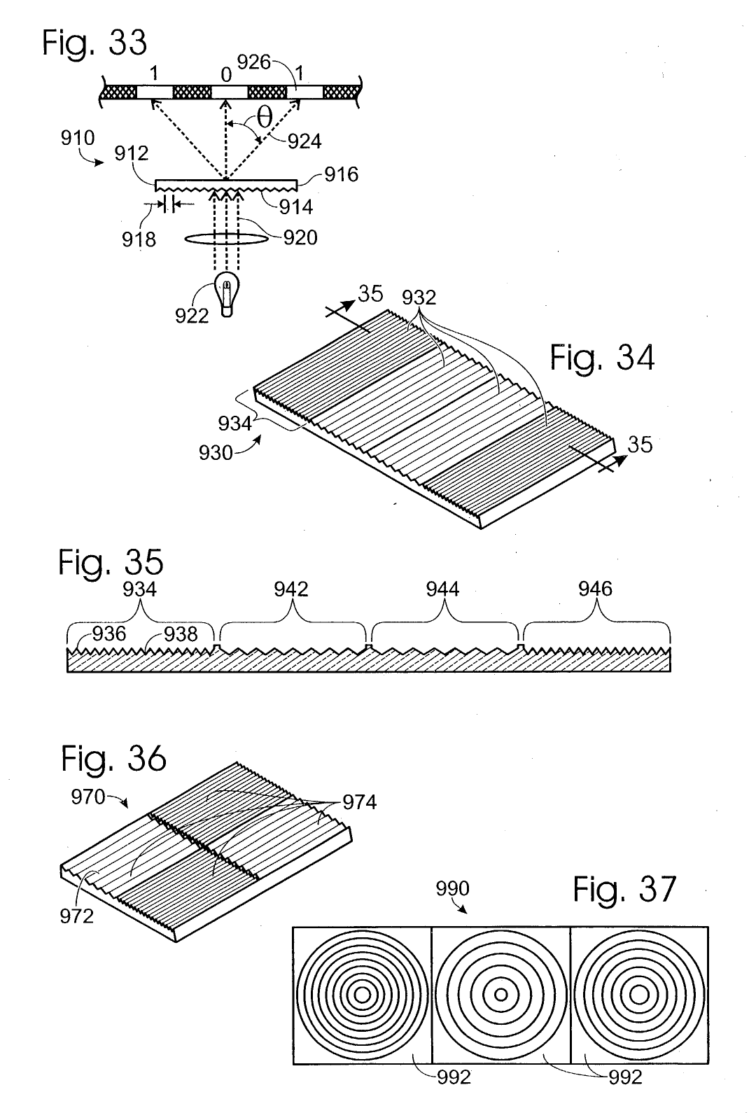

This example describes coded particles (or carriers) having an optically detectable code formed by surface contours on the particles and methods for making and using such particles; see FIGS. 33-51.

Particles, such as microparticles, have numerous uses as fillers, tracers, carriers, or tags. However, the size of particles may create some difficulties during production of the particles. In particular, placing identifiable features on a particle, particularly a microparticle, may be costly or impractical. For example, forming a particle of separate, optically distinguishable parts may require that the parts be produced separately and then united as a composite by fusion of the parts. Accurate joining and alignment of the parts during fusion may be very time consuming and technically difficult. Thus, a particle formed integrally, but including an optically detectable code, would be useful in many particle applications.

The invention provides coded particles that form an optically detectable code at least partially through surface contours present on the particles, and methods for producing and using these coded particles. Plural surface contours may be disposed positionally on each particle to form a positional code. In some embodiments, the surface contours include one or more interference filters formed as diffraction gratings on the particle, with each interference filter conferring a detectable optical property to a region of the particle and thus forming a spatial code. Each interference filter may carry optically determinable information, for example, encoded in the position, shape, size, and/or optical properties of the filter, among others.

Optically detectable surface contours may be formed on particles concomitant with production of the particle, for example, by soft lithography using replica molding. Therefore, coded particles may be formed integrally, with a complete optically detectable code, circumventing a need for formation of separate code components as individual units and/or a requirement for forming the code during a distinct step. As a result, the invention may allow formation of coded particles using methods distinct from other approaches described in this Detailed Description.

The following sections describe further aspects of the invention, including (4.1) optically detectable surface contours, (4.2) surface-contour codes, (4.3) producing particles with surface-contour codes; (4.4) reading surface-contour codes, (4.5) selected embodiments I, and (4.6) selected embodiments II.

4.1. Optically Detectable Surface Contours

Optically detectable surface contours generally comprise any surface contour, other than an edge or comer, that detectably alters a property of light. Surface contours may comprise any optically distinguishable deviation from a substantially planar or convexly contoured exterior surface of a particle. In addition surface contours may comprise any other optically distinguishable deviations from substantially homogenous or continuously random or amorphous structure of the exterior and/or interior of the particle. Thus, the surface contours may include ridges, grooves, dimples, bumps, and/or any other optically detectable structure. In addition, the surface contours may have a distinct shape, such as a symbol. The surface contours also may be photonic crystals formed at the surface but extending inward or positioned at least substantially in the interior. These crystals may be manufactured by lithography or colloidal assembly to produce a rectangular or spherical lattice structure, among others.

In some embodiments, the surface contours may include interference filters. An interference filter generally comprises any structure that reflects or transmits incident light to create a defined pattern of destructive and constructive interference of the incident light. An example of an interference filter suitable for use in the invention is a diffraction grating. Optical properties of a diffraction grating are shown in FIG. 33 and indicated generally with the numeral 910. A diffraction grating 912 may be formed as a series of generally equally spaced, and parallel, grooves or ridges 914 on a particle 916. The distance between adjacent grooves or ridges is defined as the spacing, d, shown at 918. When incident light 920 from a light source 922 strikes the diffraction grating, the incident light is transmitted (or reflected) by the grating, with maxima positioned according to the equation: dsinθ=mλ. In this equation, d is the spacing 918 defined above, θ defines the angular positions 924 relative to normal from the surface of the particle at which constructive interference occurs, λ is the wavelength of incident light, and in is the order for each of plural intensity maxima, such as one of the first order maxima shown at 926. Thus, as shown in FIG. 33, 0^{th} order light (m=0) is not diffracted, first order light (m=1) is diffracted constructively along an angle θ based on the above equation., and so on. The width of the maxima generally decreases inversely to the number of grooves or ridges in the grating. Therefore, by using a defined grating spacing and monochromatic light, the angular position of each intensity maximum may be calculated. By altering the grating spacing, light emission maxima for a particular wavelength of incident light will assume distinct angular positions relative to the surface of the filter, and thus will be detectably distinguishable, as exemplified below. In some embodiments, the average spacing within a diffraction grating may be between about 100 nm and 5 μm. Each interference filter on a particle may provide an optically detectable surface contour that is defined according to the position, shape, size, and/or optical properties of the interference filter.

4.2. Surface-Contour Codes

A surface-contour code generally comprises any optically distinguishable surface contour or set of contours formed on the surface of a particle. The surface-contour code may be formed from a positionally disposed set of contours. For example, the surface-contour code may be formed from a set of diffraction-grating interference filters on a particle, where the set includes one or more filters, with each filter having a distinguishable position within the particle. The distinguishable positions may be defined relative to each other, relative to the particle, or may be arbitrarily distributed. In addition, the positions may be at least substantially nonoverlapping. The interference code may define the overall code or may be used in combination with other detectable aspects of a particle to define the code. For example, an interference code may be used in conjunction with other detectable positional or nonpositional features of a particle to define the code. Furthermore, an interference code may be disposed on a coding portion of a particle, substantially nonoverlapping with a noncoding or assay portion.

Exemplary coding and noncoding portions are shown and described elsewhere in this Detailed Description.

4.3. Producing Particles with Surface-Contour Codes

Surface-contour codes may be produced during and/or after particle production. Surface-contour codes formed after particle production may include surface modification by printing, etching, scratching, stamping, and/or any other process that alters a surface contour of a particle. For example, a diffraction grating may be formed by etching or scratching grooves in the surface of a particle. Alternatively, ridges may be formed on the surface of a particle, for example, by printing using a lithographic process, such as soft lithography.

Surface-contour codes also may be formed during particle production. By using a mold to define the particle shape and surface structure, a desired surface contour or set of contours may be produced. Such molds may be formed using soft lithography processes or any other suitable molding or replica molding process. Diffraction gratings may be formed on particles by the presence of a series of grooves/ridges on the mold.

4.4. Reading Surface-Contour Codes

Surface-contour codes may be read using any detection system capable of detecting the surface contours of the code, including optical techniques and surface probe techniques, among others. The optical techniques may read the code by measuring the intensity, wavelength, polarization, pattern, and/or other properties of light transmitted, reflected, and/or absorbed by the code using any suitable process, including diffraction, luminescence (including photoluminescence (e.g., fluorescence and phosphorescence) and chemiluminescence), absorption, scattering, and/or reflection, among others. The optical techniques also may measure similar quantities using wave-like particles, such as electrons, for example, in scanning electron microscopy (SEM). The surface probe techniques may read the code by monitoring the interaction of a probe with the surface using any suitable process, including atomic force microscopy (AFM), scanning tunneling microscopy (STM), near-field scanning optical microscopy (NSOM), magnetic force microscopy (MFM), and/or electric force microscopy (EFM), among others. The same and/or different techniques may be used to read the code and to read the associated assay result.

4.5. Selected Embodiments I

The following sections describe particles (or carriers) with exemplary diffraction grating interference codes, and methods of making these particles and reading the interference codes. These embodiments are included for illustration and are not intended to limit or define the entire scope of the invention. For clarity, the drawings may exaggerate the relative sizes and scales of the surface contours.

4.5.1 Interference Code 1

This section describes a generally planar coded particle 930 with a positional interference code; see FIG. 34. Particle 930 includes a set of four optically detectable interference filters 932. Interference filters 932 are formed as diffraction gratings on the surface of the particle. Each filter occupies a region or grid field 934 on the surface. In this case, a linear array of four grid fields are disposed across a surface of the particle. As shown in FIG. 35, grid field 934 may be formed as a series of ridges 936 flanked by grooves 938, with a ridge-to-ridge and groove-to-groove spacing of “d”, referred to hereafter as a grid spacing. Each grid field may have any suitable number of ridges with any desired grid spacing that provides a measurable optical property. For example, in FIG. 35 each grid field may have a distinct spacing within each of grid fields 934, 942, 944, 946.

4.5.2 A Particle with an Interference Code

This section describes exemplary dimensions and materials for a particle with an interference code. A particle that is 80 μm×80 μm×10-20 μm may be prepared by soft-lithography. The 80 μm×80 μm face may be composed of four 20 μm×80 μm fields, each composed of a diffraction grating pattern that defines a grid field with a distinguishable optical property. When this particle is placed in a light path, each grid field may distinctively diffract light based on the field's diffraction grating. Therefore, it may be possible to produce coded particles from a single piece of plastic (or other material), instead of requiring, for example, a composite of plastics having distinct colors.

4.5.3 Interference Code 2

This section describes a two-dimensional interference code on a particle; see FIG. 36. Grid fields may be arranged on a particle in any suitable arrangement. For example, as shown in coded particle 970 in FIG. 36, interference filters 972 may have positional information in two dimensions of a planar particle, for example, by forming rows and columns of grid fields 974, or by another regular or random arrangement.

4.5.4 Interference Code 3

This section describes a coded particle 990 showing the use of circular diffraction gratings 992 to form an interference code; see FIG. 37. Here, the grooves/ridges of the grating form concentric rings with a fixed spacing. Circular diffraction gratings may produce circular intensity maxima and thus may have less orientation dependence than linear gratings. Other nonlinear shapes also may be suitable such as elliptical, rectangular, triangular, and polygonal, among others.

4.5.5 Interference Code 4

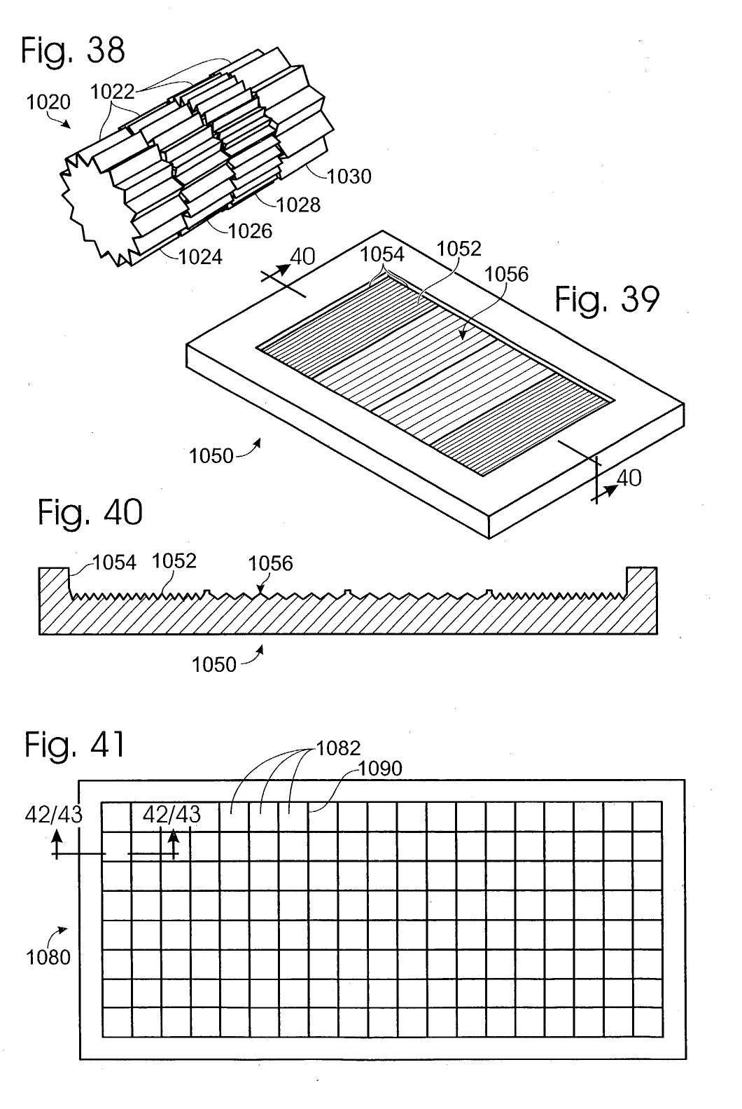

This section describes a nonplanar interference code in a generally cylindrical particle 1020; see FIG. 38. Here, each interference filter 1022 is disposed circumferentially on the particle, producing a banded pattern of grid fields 1024, 1026, 1028, 1030. In other embodiments, the grid fields may extend parallel to the long axis of the particle and/or grooves/ridges may extend circumferentially.

4.5.6 Molding an Interference Code

This section describes a mold 1050 that may be used to form an interference code on a particle, in this case, particle 930 of FIGS. 34 and 35; see FIGS. 39 and 40. Mold 1050 includes a recess 1052 dimensioned to receive precursor material for particle 930. Generally, the precursor material is any material that may be converted within the mold to a generally stable shape conforming to the mold. Examples of precursor material are a pre-polymer that will form a plastic, or a ceramic, molten, or sol-gel material that will form a glass. Recess 1052 is defined by walls 1054 and a floor 1056. The floor includes spaced grooves/ridges that define a complementary interference filter or set of filters on the molded particle.

Molds may be formed of glass and include a glass diffraction grating as floor 1056 to direct molding. Alternatively, molds may be made from a plastic. Furthermore, molds may be prepared from lithographed silicon or metal foils.

4.5.7 Forming Particles using a Molding Matrix

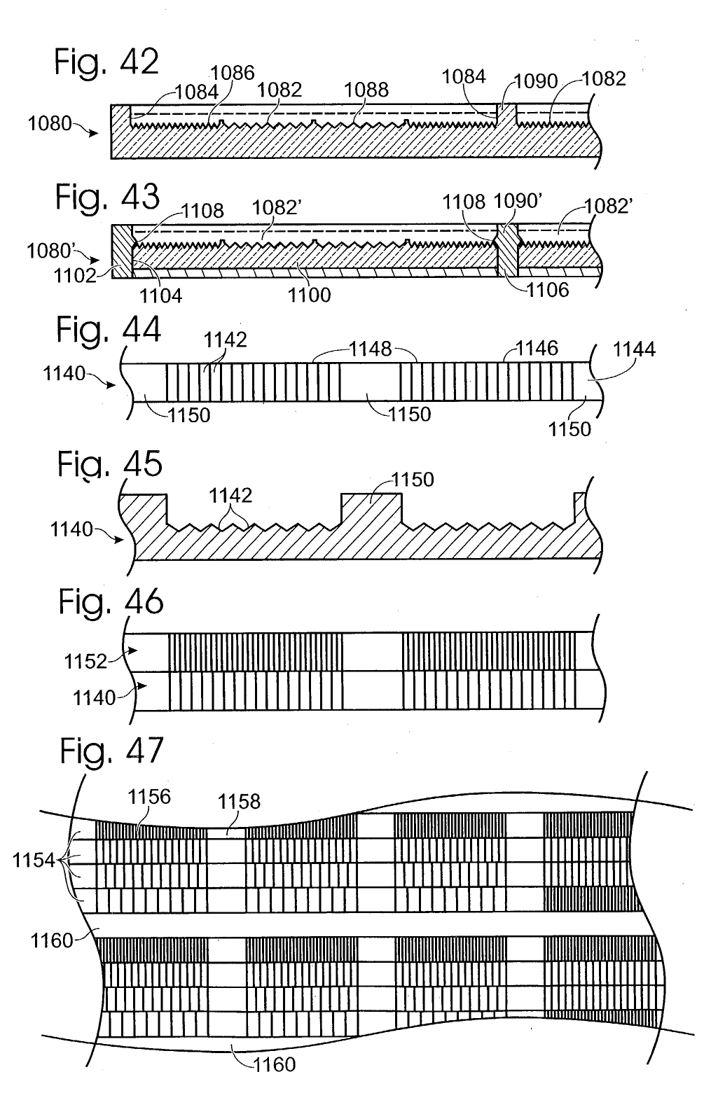

This section describes a method that may be used for the bulk manufacture of particles with interference codes using a molding matrix 1080 or 1080′; see FIGS. 41-43. The molds in the matrix may define the same code or may define different codes. As shown in FIG. 41, molding matrix 1080/1080′ includes plural recesses 1082, with each recess defining the structure and interference code of a particle formed in the recess. As shown in FIG. 42, each recess 1082 is defined by structures similar to recess 1056 of mold 1050, as revealed by a comparison of FIGS. 40 and 42. Recess 1082 includes walls 1084 and a floor 1086 with grooves/ridges 1088. Interior walls are formed by boundary elements 1090 that extend from end to end and from side to side of matrix 1080 to define rows and columns of molds within the molding matrix. Boundary elements 1090 act as spacers that may take the form of ridges or other structure that physically separate particles from each other as they form. Spacer 1090 may extend above the future top molded surface of the particle to prevent physical linkage of the forming particles. The depicted embodiment of molding matrix 1080 provides the capacity to mold about 150 particles at one time. In alternative embodiments, a molding matrix may be configured to produce a much larger number of particles at one time, such as a 50 by 100 matrix capable of forming about 5,000 particles at once.

4.5.8 Molding Matrices with Modular Molds

This section describes a molding matrix with modular molds; see FIG. 43. Specifically, molding matrix 1080′ may be formed of individual particle molds. In contrast to molding matrix 1080 shown in FIG. 42, individual particle molds, such as mold 1100, may be provided as molding modules that are inserted into a molding matrix frame 1102. Walls 1104 of each molding recess may be provided by frame 1102 and spacers 1106. Alternatively, each individual mold may provide walls around its perimeter. Spacers 1106 may be used in conjunction with frame 1102 to fix the position of molds 1100 by locking them in place, for example, using a flange 1108 to retain an edge of the mold. Use of modular molds in a molding matrix allows the matrix to form any desired combination of coded particles, based on available molds.

Molding matrix 1080′ described above may be likened to an old style printing press in which lead type molds are put in place and locked into a frame. In a similar manner, pools of molds for each class of particle may be prepared, loaded, and reused in molding matrices, as required. This approach may generally require the overhead cost of producing a predetermined number of different mold pools, one pool for each class of particle.

4.5.9 Molding Matrices with Modular Grid Fields

This section describes the use of grid-field modules to form molds within a molding matrix; see FIGS. 44-47. Each row or column of molds within a molding matrix may be formed by aligned layers of modular grid-field molds. Each layer may define a line of grid fields at a similar position in each particle of the row or column. For example, a row of molds may be designed to direct formation of a row of particles with four linearly disposed grid fields, such as found in particle 930 of FIG. 34. In this case, layer one may define the first grid field for each particle of the row, layer two the second grid field for the entire row, and so on.

FIG. 44 illustrates how a grid-field mold module may be structured. Grid-field mold module 1140 may be formed from a sheet of material, such as a metal foil, a silicon wafer, or a sheet of glass, conceptually similar to a cover slip for a microscope slide. Although any thickness may be used, in some embodiments, a suitable thickness for the sheet may be about 100 μm or less. To produce grid-field mold module 1140, lines 1142 may be scribed along thin edge 1144 of the sheet, perpendicular to a face 1146 of the sheet. Generally, grid-field molds 1148 each define a mold for a grid field. Grid-field regions are scribed, interspersed with spacers 1150 that remain unscribed. Each spacer 1150 functionally corresponds to a portion of spacer 1090 of molding matrix 1080, shown in FIG. 41, and may define a portion of each particle edge. When viewed from the side, as in FIG. 45, spacers 1150 may appear as raised sections relative to each serrated grid-field mold 1148. The length of a grid-field mold is generally equal to the width of one particle. In addition, the number of grid-field molds in a sheet determines the number of particles for which the grid-field mold module may define a grid field.

Linear arrays of grid-field molds may be formed by stacking grid-field mold module 1140 with additional grid-field mold modules, such as module 1152, as shown in FIG. 46. Generally, stacked grid-field mold modules are layered, with grid-field molds and spacers aligned. In this embodiment, module 1152 defines a series of grid fields with optical properties distinct from module 1140. Each module may define grid-field molds with similar or distinct groove spacings.

A portion of a molding matrix formed from grid-field mold modules is shown in FIG. 47. In this embodiment, an assembly is formed from four grid-field mold modules 1154 placed in abutment, with grid field regions 1156 and spacers 1158 aligned. Spacers 1158 together may define two of the edges of each particle. The assembly may be sandwiched between unscribed blanks 1160 to define the other two edges of the particles.

Grid-field mold modules outlined above for FIGS. 44-47 may be produced by batch processing. For example, a number of blank sheets may be stacked in an assembly, and then scribed across all the sheets in the assembly at the same time. In this way, numerous molds for each grid-field spacing may be manufactured together and used as necessary. Although grid-field molds are exemplified as formed by scribe processing, any suitable method may be used. For example, if silicon wafers were used, grid field molds may be formed on the edges of the wafers by photolithography and etching.

The level of modularity provided by grid-field mold modules may greatly reduce manufacturing cost. As an example, using individual modular molds, such as shown in FIG. 39, 1000 different molds would generally be required to form 1000 classes of particle with distinct interference codes. However, using molds formed from grid-field modules, as few as six distinguishable grid-field mold modules that define distinct grid fields on a particle, used four at a time, may define 1000 classes of particles.

4.5.10 Forming an Array In Situ with a Molding Matrix

This section describes an in situ array formed with a molding matrix. Specifically, particles molded in a molding matrix represent a positional array of coded particles, in which the code at each position is known. Therefore, the positional coded array may be used to form a positional coded array of molecules, samples, and/or biological entities. For example, after molding, but prior to removing the particles from the matrix, the particles may be used as a support for in situ synthesis, such as ink-jet-mediated oligonucleotide synthesis. Alternatively, the positional coded particle array may be used for positional application of biological materials to individual coded particles. For example, the biological materials may be libraries of modulators, oligonucleotides, nucleic acids, peptides, ligands, proteins, tissues, or cells. Application may be carried out be any suitable approach, such as by spotting an aliquot of each material, for example, with a robotic system. Such an approach may be more controlled than with standard positional arrays, in that spacer width may be optimized to allow sufficient deposition without cross-contamination. Similarly, particles at certain coordinate positions in the molded array may be used as “buffer spaces” and discarded. The approach of using matrices containing different coded particles followed by in situ oligonucleotide synthesis or positional deposition of materials may be efficient methods for manufacturing diagnostic kits.

The positional synthesis or deposition approach outlined above places a material of interest on a side of a particle opposite from the interference code. However, appropriate design of a particle, for example, by forming a thin, translucent particle out of plastic should allow both reading the code and measuring a characteristic of the material of interest.

4.5.11 Producing Cylindrical Particles having an Interference Code

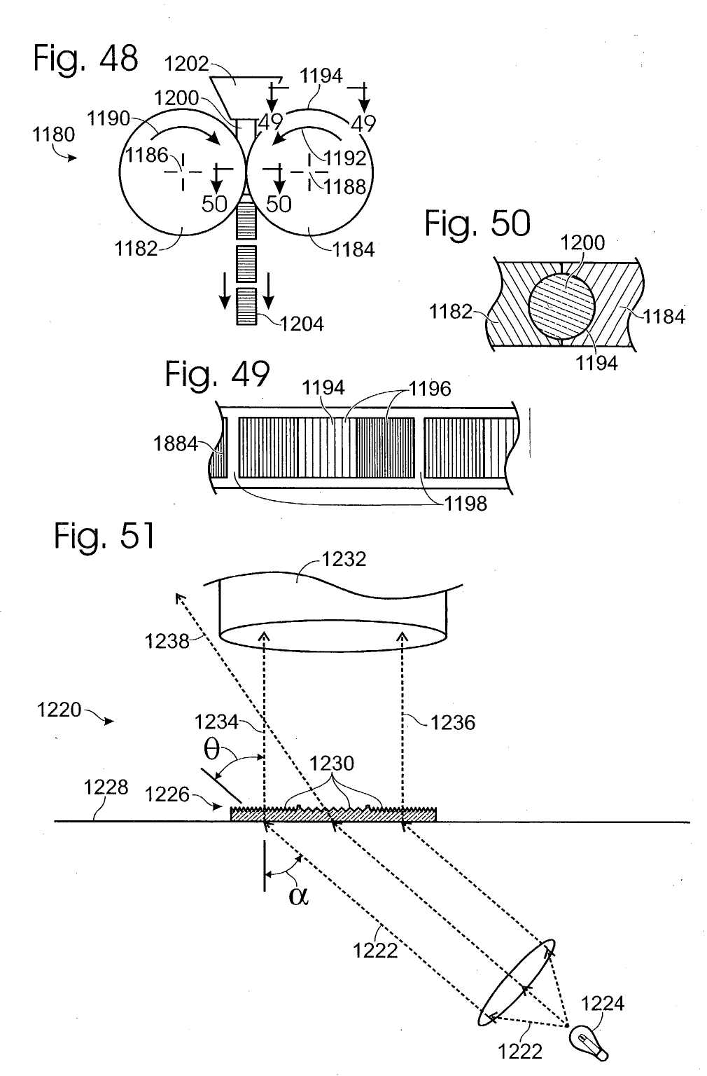

This section describes methods for producing cylindrical particles with an interference code, such as particle 1020 of FIG. 38, by molding; see FIGS. 48-50. A suitable molding device may use opposing, rotating molds formed on a semi-circular rim of rotating wheels. Such a wheel and rim combination may be found, for example, on a pulley wheel, such as the type used to guide an O-ring belt. The opposing molds each may form half of a cylindrical particle and half of each grid field.

An embodiment of an apparatus for forming cylindrical particles is shown in FIG. 48. Rotary mold 1180 includes two rotating wheels, 1182 and 1184, which synchronously rotate about axes 1186 and 1188, respectively, in opposing directions, as indicated by arrows 1190 and 1192. Each wheel includes a semi-circular notch 1194, as shown in FIGS. 49 and 50, which is circumferentially disposed around each wheel. FIG. 49 shows how each notch is engraved with a series of grid field molds 1196 and spacers 1198 in the form of ridges. Each set of grid field molds places a code on a forming particle and the spacers define the ends of the particles as they are being formed. Each set of grid field molds produces an interference code on a semi-circular half of a cylindrical particle. Two similarly engraved wheels are set together in register, notch-to-notch, such that the two notches are opposed to each other and define a circular/cylindrical path, as shown in FIGS. 49 and 50.

A moldable material 1200, such as a plastic, then may be extruded from a reservoir 1202 into a circular opening formed between opposing notches, as seen in FIG. 50, and as the wheels turn, the moldable material is embossed with the grid field patterns on the notches. The stream of moldable material is cleaved via the ridges into individual cylindrical particles 1204 with interferences codes, as shown in FIG. 48.

In alternate embodiments, each wheel may have multiple, parallel notches disposed axially relative to each other, so that the notches pair with opposing notches to simultaneously form multiple particles. Each pair of opposing notches may be fed by a separate extruded strand of moldable material.

The process may not be limited to producing one class of particle at a time. Since the end products are separate particles, each with a code, multiple classes of particles may be made at the same time and later separated, for example, by a flow-type sorting mechanism.

4.5.12 Reading an Interference Code

This section describes a system 1220 for reading interference codes; see FIG. 51. In this embodiment, interference filters on a particle are distinguishably identified by measuring the intensity of diffracted light at particular wavelengths and angular positions for each filter. This may be carried out by using a nonorthogonal angle of illumination, angle of detection, or both. In system 1220, monochromatic light 1222 is emitted from a light source 1224 and directed toward a coded particle 1226 carried on a stage 1228 and having an interference code formed by three grid fields 1230. Light 1222 is directed at the interference fitter at an angle α a relative to normal. Incident light is diffracted constructively by each grid field at angles of θ, given by the equation dsinθ=mλ described above. Detector 1232 is positioned so that it detects only light transmitted substantially normal to the stage/particle. When α is substantially equal to θ of an intensity maximum, detector 1232 will detect light, as shown for diffracted light 1234, 1236 transmitted from the outer two grid fields. In contrast, when the grid field spacing does not diffract light constructively toward the detector, as shown for distinctly diffracted light 1238 from the middle grid field, the detector will receive detectably less diffracted light from this middle region of the particle. Therefore, by adjusting the angle α at which light is directed to the particle and/or the wavelength of light used, optical properties are assigned to each grid field.

System 1220 generally relies on light being directed orthogonal to the grooves/ridges of a diffraction grating for predicted diffraction to occur. Thus, system 340 may need to be modified to accommodate various nonorthogonal orientations of linear diffraction gratings produced by random particle distributions. One of the following modifications may prove suitable. (1) The particle may be rotated mechanically (or, in some cases, by a magnetic field) to provide an orthogonal disposition of incident light and the diffraction gratings. (2) The orientation of the particle may be determined and then the light source moved or one of plural alternative light sources used that is disposed at the proper position so that the light source is orthogonal to the grating. (3) The light may be directed normal to the stage and the detector placed to receive light diffracted at an angle. (4) Nonlinear diffraction gratings may be used, such as the circular interference filters of FIG. 37.

4.5.13 Internal Codes

The code also may be an internal code rather than, or in addition to, a surface contour. The internal code may be formed via modification of the internal structure and/or composition of the particle. For example, the particle may include an internal portion that has been lithographed to be a matrix, a lattice, or a honeycomb, among others.

4.6 Selected Embodiments II

This section describes further aspects of the invention, as set forth in the following numbered paragraphs:

1. A particle with a surface code, comprising: a particle adapted for supporting biological samples and having a contoured surface, the contoured surface providing at least one optically detectable feature.

2. The particle of paragraph 1, where the contoured surface includes plural at least partially overlapping surface contours, each surface contour providing at least one optically detectable feature.

3. The particle of paragraph 1, where the at least one optically detectable feature includes an interference filter.

4. The particle of paragraph 3, where the interference filter is a diffraction grating.

5. The particle of paragraph 1, where the particle is formed integrally.

6. The particle of paragraph 1, where the contoured surface includes plural at least substantially parallel grooves.

7. The particle of paragraph 6, where the grooves are separated by an average spacing, and the average spacing is between about 100 nm and about 5 μm.

8. The particle of paragraph 6, where at least some of the grooves are selected from the group consisting of at least substantially linear and at least substantially circular.

9. The particle of paragraph 2, where the plural contours are disposed in an at least substantially linear array on the surface.

10. The particle of paragraph 2, where the plural contours are disposed in a two-dimensional array on the surface.

11. The particle of paragraph 1, where the particle is at least substantially planar.

12. The particle of paragraph 1, where the particle is at least substantially cylindrical.

13. A method of producing a particle with a detectable code, comprising: forming a particle in a mold, where the particle is adapted to support a biological sample, and the mold defines a contoured surface on the particle, the contoured surface having a detectable optical property.

14. The method of paragraph 13, where the contoured surface includes plural at least partially nonoverlapping surface contours.

15. The method of paragraph 13, where the contoured surface includes an interference filter.

16. The method of paragraph 14, where the plural surface contours include a diffraction grating.

17. A method of producing plural particles having interference codes, comprising: forming plural particles in a molding array, the molding array including plural molds, where each of the plural molds defines plural interference filters on one of the plural particles, the plural interference filters are arrayed to provide one of the interference codes, and the one interference code is adapted to at least partially identify one of the plural particles.

18. The method of paragraph 17, where the plural molds are removable from the molding array.

19. The method of paragraph 17, where the molding array includes spacers that define edges of the particles.

20. The method of paragraph 17, where the plural interference filters are defined by plural grid field modules, each grid field module defining at least one of the plural interference filters and including plural grid fields spaced to define at least one other interference filter in a linear array of the plural particles.

21. A method of associating a material with a coded particle, comprising: producing plural particles with interference codes according to paragraph 17; disposing the material on one of the plural particles, where the one particle includes a known one of the interference codes, thereby linking the material to the one interference code.

22. The method of paragraph 21, where the material is selected from the group consisting of synthetic oligonucleotides, nucleic acids, peptides, ligands, drugs, proteins, tissues, and cells.

23. The method of paragraph 21, where the material is disposed based on a position of the one particle within the molding array.

24. The method of paragraph 21, where the material is disposed by in situ synthesis.

25. A particle with a surface code, comprising: a particle adapted for supporting biological samples and having a surface contour, the surface contour providing at least one optically detectable feature.

26. The particle of paragraph 25, where the surface contour extends at least substantially below the surface of the particle.

27. The particle of paragraph 25, where the surface contour includes photonic structures.

28. The particle of paragraph 25, where the photonic structures are selected from the group consisting of pores, spheres, lattices, and honeycombs.

29. The particle of paragraph 25, where the surface contour is a nonhomogenous composition extending into the particle.

### Example 5

### Particles with Topographic Codes

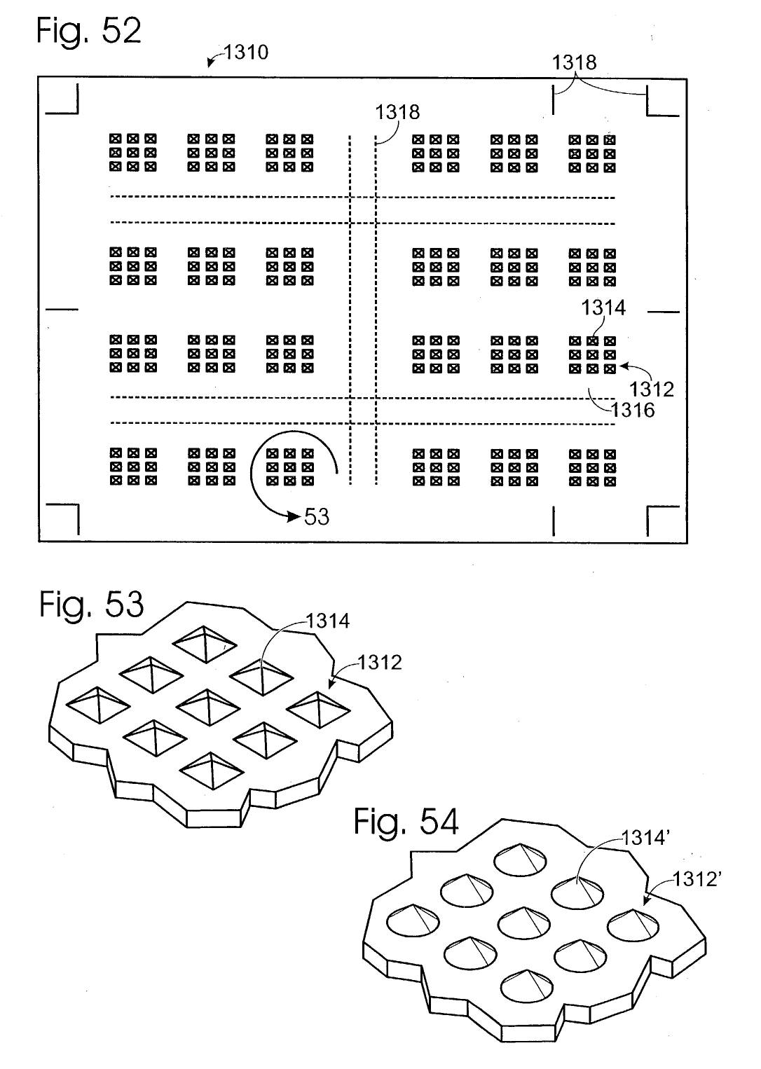

This example describes systems, including methods and apparatus, for making and using particles (or carriers) with topographic codes, particularly for multiplexed analysis of biological systems; see FIGS. 52-59.

Coded microparticles provide a support structure for the multiplexed analysis of biological systems. The production and use of coded microparticles for the detection, analysis, and quantification of analytes has been described above. In some embodiments, an optically distinct material in the form of a thin film is attached at discrete positions of a microparticle surface to achieve optical contrast at these positions. For example, Example 2 above (see FIG. 26) describes a method that produces optically distinct regions at the microparticle's surface by a sequential process of photolithography, etching, thin film deposition, and polishing. The method may provide good optical contrast for either transmitted or reflected light, but may have some disadvantages, including (1) high cost and complexity of manufacturing, (2) possible incompatibility of the deposited film material with subsequent analyte analyses, (3) possible noncontrollable delamination of the thin film material due to variations in adhesion, resulting in corresponding code errors, and/or (4) consumption of the useful area of the microparticle for cell growth, producing a corresponding reduction in potential signal, such as fluorescent response. Thus, there is a need for simpler, more efficient systems for forming codes on microparticles.

The invention provides systems, including methods and apparatus, for making and using particles with topographic codes, also referred to as surface relief codes. These topographic codes may include any detectable surface relief features, such as recesses and protrusions, defined by the surface of the particle. The surface relief may produce regions of the particle that show altered optical properties. This surface relief may modify the properties of incident light distinctively, thus producing a nonuniform spatial pattern of light detected from the particle. The spatial pattern forms a distinguishable code that relates information about the particle, a supported sample, a method of analysis, or the like, thus allowing multiplexed analysis in nonpositional and positional arrays.

Surface topography or surface relief may be formed by any suitable method, including stamping, molding, and etching, among others. In some embodiments, the code is stamped or imprinted in the particle by controlled deformation of a surface of a particle precursor or progenitor material, using a die. In imprinting, the die has a topography that shapes a generally complementary topography of surface relief on the resulting particle.

The systems of the invention may offer a number of potential advantages for forming coded particles relative to other approaches. For example, these systems may facilitate higher manufacturing throughput, with lower cost and higher yield. In addition, these systems may allow the particles to be produced from a single precursor material, without the addition of coatings or films, or the fusion of distinct components. As a result, the chemical and biological properties of the particle may be defined more uniformly by a single particle material, rather than plural particle materials distributed nonuniformly. Furthermore, coded particles produced according to the present invention may increase available surface area in forming the code, thus increasing effective particle sample capacity, including available area for cell growth. As a result, particles of the invention may provide a greater signal, such as fluorescence response.

Further aspects of the invention are described in the following sections: (5.1) particles; (5.2) topographic codes, including (1) imprinted features, (2) molded features, and (3) code elements; (5.3) dies and molds; (5.4) forming topographic structure; (5.5) reading topographic codes; (5.6) assays with particles having topographic codes; (5.7) selected embodiments I; and (5.8) selected embodiments II.

5.1. Particles

Particles may be formed of any material capable of forming a detectable surface topography. For example, particles may be formed by alteration of a precursor material's surface structure, such as by pressure, laser ablation, or chemical etching. When formed by pressure, the particles may be produced from any material capable of receiving and retaining an imprint, and may be formed of a malleable or plastic material. The material may be a thermoplastic material and may be colorless and/or transparent. Thermoplastic materials include any resin that shows increased deformability when heated. Exemplary materials may include acrylates, such as polymethyl or polyethyl methacrylates (e.g., PMMA or PEMA, among others), acrylonitrile/methyl methacrylate, etc.; polycarbonates; polyolefins, such as polypropylene, polyethylene, etc.; styrenics, such as polystyrene; polysulphones; polyesters, such as polybutylene terephthalate, polyethylene terephthalate, polypropylene terephthalate, and the like; polyimides; polyphenylenes; vinyl-based resins; and composites thereof, among others. Alternatively, particles may be molded from any material that undergoes controlled solidification, such as conversion from a pre-polymer to a polymer, or from a fluid (e.g., molten) to a solid state among others.

Particle size may be determined based on the application and ease of handling, among others. A thickness for particles with surface relief codes may be about 10-200, 20-150, or 50-100 microns, and thus may be formed from sheets or films of material with a corresponding thickness.

Further aspects of particles shapes, sizes, and materials are described elsewhere in this Detailed Description, such as Sections II-IV and X, and in the patent applications identified above under Cross-References and incorporated herein by reference, particularly U.S. patent application Ser. No. 09/694,077, filed Oct. 19, 2000; and U.S. patent application Ser. No. 10/120,900, filed Apr. 10, 2002.

5.2 Topographic Codes

A topographic or surface-relief code is defined by surface relief features. The features may be formed on particles by any suitable process, including stamping an imprint in a particle precursor material; molding surface relief, for example, by soft lithography; and/or removing material from or depositing material to a particle surface, for example, by laser or chemical etching or crystal growth. In some cases, topographic codes may include or be combined with nontopographic codes, for example, to identify particles and/or associated samples/reagents or assays. Suitable nontopographic codes are described elsewhere in this Detailed Description.

The surface relief may include recesses and/or protrusions. The recesses and/or protrusions may be in the form of grooves, ridges, dimples, bumps, pyramidal or conical depressions or relief structures, frustocones, hemispheres, symbols, complex shapes, and the like. Thus, the cross-sectional shape of a surface relief feature may be square, rectangular, round, elliptical, parabolic, polygonal, arcuate, curvilinear, and so on. One or plural surface relief may form a topographic code. Surface relief features are described in more detail in, for example, Section V and Example 1 above.

5.2.1 Imprinted Features

A topographic code may be formed by imprinting features on a particle surface. Imprinted features or imprints generally comprise any surface deformations formed by pressure on a particle precursor material. The pressure may be applied using any suitable mechanisms, such as a die. A precursor material may have any suitable surface, such as a generally planar or cylindrical surface, which is deformed in creating the imprint. The shape of surface relief may be determined by complementary surface relief on a die, for example, die features defined by anisotropic or isotropic etching of monocrystalline silicon.

5.2.2 Molded Features

A topographic code also may be formed by molding features on a particle surface, for example, by introducing particle precursor material, generally in a liquid form, into a mold, and then solidifying or hardening the precursor material. Forming surface relief by molding is described in more detail elsewhere in this Detailed Description, particularly in Example 4 above.

5.2.3 Code Elements

The topography on a particle may define one, but more typically, plural distinct code elements. The code elements, generally corresponding to distinct surface relief features, may produce a code by any combination of position, number, shape, size, height, and/or optical properties of the elements. Position may be absolute and/or relative, that is, the position of each element may be determined relative to the particle and/or to each other, and may be predefined or random. Size, similarly, may be measured absolutely or relative to the particle or a reference structure on the particle, among others. Optical properties suitable for code elements may include any measurable change in spectroscopic properties determined by the code elements, including absorption, reflection, refraction, diffraction, optical rotation, dichroism, and/or so on. In some embodiments, the optical properties imparted by a code element may include changes in light transmission, such as divergent or convergent refraction of light.

Codes and properties of code elements that may be suitable are described elsewhere in this Detailed Description, particularly in Section I above.

5.3. Dies and Molds

Particle topography may be formed using any suitable mechanism, including dies and/or molds, among others. A die generally comprises any structure having a set of surface relief that imparts a generally complementary topography to a particle precursor material in response to contacting pressure. In contrast, a mold generally comprises any structure that constrains a liquid precursor material in a shape generally corresponding to the surface relief of the mold as the precursor material solidifies. Dies and molds may have any suitable composition, size, shape, and/or number of surface relief features.

Dies and molds may be formed of any suitable materials. Dies may be formed of any material that is harder than the particle precursor material under the conditions at which die and particle precursor are contacted. Suitable materials may include silicon, such as monocrystalline, polycrystalline, or amorphous silicon, or combinations thereof, a metal or metal alloy, such as steel, aluminum, brass, etc; a plastic; and/or a glass or ceramic, among others. Molds may be formed of metal, glass, ceramics, and/or plastics, among others. Both molds and dies may be formed of elastomeric materials, such as those used in soft lithography. An embodiment of a mold formed by soft lithography is described above in Example 2, particularly in relation to FIG. 27.

Dies and molds may have a size and shape that corresponds to one or more particles. Thus, a die or mold may produce a topographic code on only a single particle, on two particles, or generally on many particles at the same time. Dies and molds may impart additional noncoding features to a precursor material, such as orientation marks, symbols, lines, or marks for partitioning the precursor material into plural particles, and so on. Dies and molds may form plural particles that abut and/or include nonparticle spacer material.

Dies and molds have surface relief that is generally complementary to the topography formed on particles. Thus, the topography of a die or mold may include one or more surface relief features, with each feature forming a corresponding feature on a particle. Accordingly, each feature may be a recess or a protrusion, and may be in the form of a groove, ridge, dimple, bump, pyramid, cone, frustocone, hemisphere, symbol, complex shape, and/or a combination thereof, among others. In addition, the cross-sectional shape of a feature may be square, rectangular, round, elliptical, parabolic, polygonal, arcuate, and/or curvilinear, among others.

Surface relief on a die or mold may be formed by selective removal, deposition, or other restructuring of die- or mold-forming materials. Thus, features may be formed by soft lithography, photolithography followed by chemical etching, laser etching, crystal growth, and/or so on.

The surface of the die or mold may be covered, after formation of the desired surface relief, with an additional layer to minimize sticking of a particle precursor-material to the die or mold surface during stamping or molding. The additional layer may include silicon oxide or nitride, teflon, parylen, etc.

5.4 Forming Topographic Structure

Topographic structures may be formed using any suitable mechanism, including stamping or molding, as described below.

5.4.1 Stamping

Topographic structure may be formed in a particle precursor material by stamping. Stamping generally comprises contacting precursor material with a die and applying pressure, generally normal to the contact surface. The pressure may be applied by the die on the precursor material, by the precursor material on the die, or both. The pressure may be generated by any suitable force, including gravity and mechanical forces such as those exerted by a motor, a spring, gas, etc.

The particle precursor-material may be softened prior to, and/or during, contact with the die. Softening may be affected by any suitable mechanism, including heat, pressure, electromagnetic radiation, particle bombardment, and/or so on. In some embodiments, heat is supplied to the precursor material, either by physical contact with a heated die or a platform that holds the precursor material, or by ambient heating, such as in an oven. The temperature of the die during imprinting may be higher than the carbonization temperature of the particle precursor-material. In this case, the resulting imprints may be dull or blackened, giving the imprints optical contrast that is detectable, for example, by measuring reflected or transmitted light.

In some embodiments, surface relief of the die may be treated with an imprint modifier. Imprint modifiers include any material that alters the physical or chemical properties of imprints. Exemplary imprints modifiers include materials with distinct optical properties relating to reflection, absorption, diffraction, polarization, refraction, fluorescence, etc., such as colored or fluorescent dyes, reflective metals, paints, and/or so on; materials with distinct chemical reactivity or binding activity; and/or materials with distinct magnetic, electrical, or nuclear properties, among others. To include an imprint modifier in an imprint, the imprint modifier may be applied to the surface of die features as a liquid (for example, a paint) or a fine powder (for example, graphite, ferromagnetic particles, etc), among others, generally before contacting the die with the precursor material. When contacted with the particle precursor-material, some of the modifier may be transferred to and generally immobilized on or in the precursor material, either coating or being embedded in an imprint on the newly formed particles. Immobilizing an imprint modifier in an imprint may increase optical contrast of the imprint or provide new chemical, magnetic, electrical, or optical properties to the particle, among others, and may be used for binding samples, forming codes, orienting particles, transporting particles, etc.

Plural particles may be formed from a die during a single imprinting. The particle precursor material thus may be partitioned before, during, and/or after the imprinting process. When partitioned before imprinting, the precursor material may be affixed temporarily to a substrate to maintain position. Partitioning during imprinting may occur with die protrusions that at least partially form a perimeter for the particle. Partitioning before or after imprinting may be conducted using any separation method. These methods may include (1) mechanical means, such as a sharp cutting edge or punch, (2) manual means, such as tearing, (3) chemical means, such as etching, and/or (4) optical means, such as laser cutting. The partitioning may be facilitated using any suitable mechanism, including guidelines, perforations, and/or scoring, formed before, during, and/or after imprinting.

5.4.2 Molding

Topographic structure also may be molded, for example, using a single mold or an array of molds. Many aspects of molding and stamping topographic codes may be similar, including partitioning, surface relief, particle size and shape, and so on. Further aspects of molding topographic structure are described elsewhere in this Detailed Description, particularly Example 4.

5.5 Reading Topographic Codes

A topographic code is read to determine information about the particle, its manipulation, and/or a sample/reagent supported by the particle. The topographic code may be read by any detection system capable of detecting surface relief, including optical techniques and surface probe techniques, among others. The same and/or different techniques may be used to read the code and to read the associated assay result.

The optical techniques may read the code by measuring the intensity, wavelength, polarization, pattern, and/or other properties of light transmitted, reflected, and/or absorbed by the particle using any suitable process, including refraction, diffraction, luminescence (including photoluminescence (e.g., fluorescence and phosphorescence) and chemiluminescence), absorption, scattering, and/or reflection, among others. The optical techniques may measure similar quantities using wave-like particles, such as electrons, for example, in scanning electron microscopy (SEM). In some embodiments, at least some of the surface relief of the topographic code refracts incident light divergently to produce regions of decreased light transmission in a corresponding image of light transmitted by the particle.

The surface probe techniques may read the code by monitoring the interaction of a probe with the surface using any suitable process, including atomic force microscopy (AFM), scanning tunneling microscopy (STM), near-field scanning optical microscopy (NSOM), magnetic force microscopy (MFM), and/or electric force microscopy (EFM), among others.

Further aspects of reading a topographic code are described below in section 5.7.3.

5.6. Assays with Particles Having Topographic Codes

Particles with topographic codes may be used to support samples for any suitable analysis using positional or nonpositional arrays. Examples of suitable biological analyses are described elsewhere in this Detailed Description and in the patent applications identified in Cross-References and incorporated herein by reference, particularly U.S. patent application Ser. No. 09/694,077, filed Oct. 19, 2000; and U.S. patent application Ser. No. 10/120,900, filed Apr. 10, 2002.

5.7. Selected Embodiments I

The following sections describe selected aspects and embodiments of the invention, including methods for forming particles having topographic codes, particularly imprinted codes, and methods for reading the topographic codes. These embodiments are included for illustration and are not intended to limit or define the entire scope of the invention.

5.7.1 Exemplary Dies

This section describes exemplary dies for forming imprinted particles; see FIGS. 52-54.

FIG. 52 shows a die 1310 used to produce a particular code pattern. Over the die surface, the die includes at least one group 1312 of features 1314 that will form the code pattern. Group 1312 (or plural groups) plus some surrounding area 1316 define the target particle size. The die may have any number of feature groups 1312. Here, thirty groups of nine features each are shown on die 1310. Groups 1312 may be located in rows and columns, thus simplifying separation of imprinted particles by cutting. The die also may have noncoding features 1318, such as lines or dots, among others, which may be used as alignment marks or perforations for particle separation. Die 1310 is manufactured using known methods of micro-machining, some of which are described below in Section 5.7.2.

FIGS. 53 and 54 show magnified views of exemplary die features. Each feature 1314 of group 1312 may be formed as a protruding pyramid (see FIG. 53). Alternatively, or in addition, each feature 1314′ of a group 1312′ may be formed as a protruding cone-like structure or sharpened cylinder (see FIG. 54). Features 1314, 1314′ may be mixed in one group, may be placed with any desired spacing, and/or may be arranged in any desired number of rows or columns. In addition, a feature at each code position may be present or absent as part of the corresponding code. The group of features may disposed at any position relative to the perimeter of a particle imprinted by the die, such as in the center or closer to an edge.

5.7.2 Systems for Forming Imprinted Particles

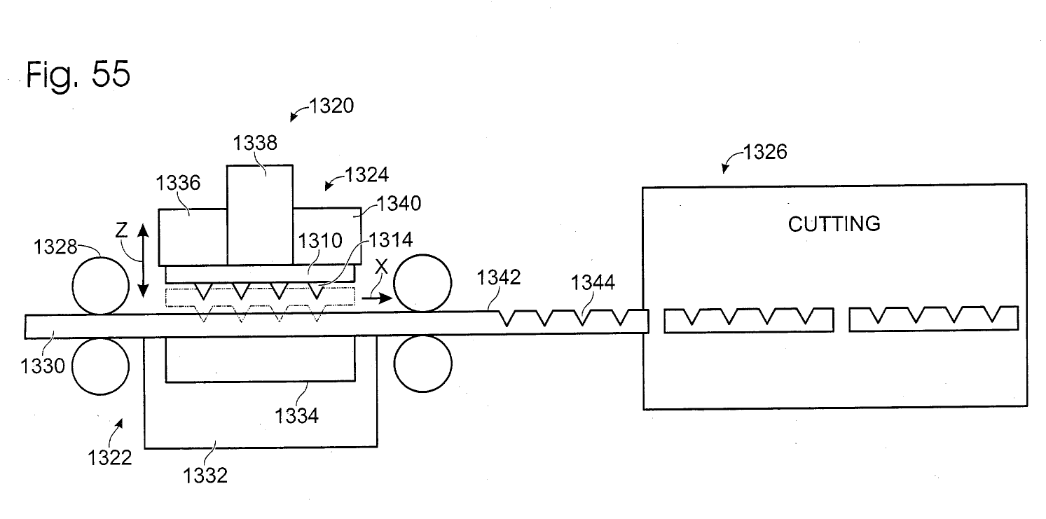

FIG. 55 shows a system 1320 for forming imprinted particles, which generally is comprises a precursor-material positioning mechanism 1322, an imprinting mechanism 1324, and a cutting mechanism 1326.

Positioning mechanism 1322 moves and supports a particle precursor material for imprinting, and advances the particle precursor material after imprinting to allow processing by the partitioning mechanism. Mechanism 1322 may include positioning structures, such as rollers 1328, which move a sheet 1330 of precursor material, such as a clear thermoplastic strip, tape, film, etc. The positioning structures move sheet 1330 parallel to its surface in one dimension, along the x-axis as indicated. Alternatively, the positioning structures may move sheet 1330 in two dimensions, for example, along the x-and y-axes, parallel to the surface of the sheet, or in three dimensions, for example, along the x-, y-, and z- axes. Positioning mechanism 1322 also includes a support structure, such as flat support 1332, which supports sheet 1330 relative to the z-axis during imprinting. Support 1332 is positioned perpendicular to the z-axis. Gravity may dispose sheet 1330 flat on support 1332. Additionally, support 1332 may include a means 1334 to attract sheet 1330 by vacuum, electrostatic, or magnetic force, among others, if gravity is insufficient to flatten and reproducibly position the sheet.

Imprinting mechanism 1324 supports, positions, and heats die 1310. Die 1310 is attached to a die holder 1336 so that the die surface is generally perpendicular to the z-axis and aligned with support 1332. Die holder 1336 may be provided with an actuator 1338 that is designed to provide controllable reciprocating movement of the die holder along the z-axis. The actuator positions the die accurately along the z-axis to effect accurate imprinting. The actuator may have a positioning accuracy of about +/− 5 microns and a motion range of greater than about 0.5 mm. Die holder 1336 also is provided with a temperature controller 1340. Temperature controller 1340 may include a heater, thermocouple and closed loop feedback control for heating and temperature stabilization. In other embodiments, a temperature controller may be provided by support 1332, or temperature control may be provided as ambient temperature control, such as with an oven that surrounds support 1332 and die holder 1336.

Cutting mechanism 1326 may be installed downstream of imprinting mechanism 1324. The cutting mechanism generally comprises any mechanism configured to divide the sheet into individual coded particles at positions between imprinted codes on sheet 1330. Additional mechanisms for cleaning or surface activation by chemical, gas, or plasma treatment also may be included.

System 1320 may be used to form imprinted particles as follows. Die 1310 is heated to a temperature higher than the softening temperature for sheet 1330. When sheet 1330 is formed of PMMA, the softening temperature is about 135° C. Actuator 1338 is moved so that the surface relief features 1314 of die 1310 penetrate sheet 1330. The penetration depth may be less than the height of die features 1314, so that the planar portion of die 1310 does not contact sheet 1330. After imprinting, the actuator is raised to its original position. As a result of imprinting, top surface 1342 of sheet 1330 has recesses 1344 with a shape and size corresponding to the portion of die features 1314 that penetrated sheet 1330. Rollers 1328 then advance sheet 1330 a distance that is equal to or greater than the length of die 1310, and imprinting is repeated.

5.7.3 Exemplary Optical System for Reading Codes

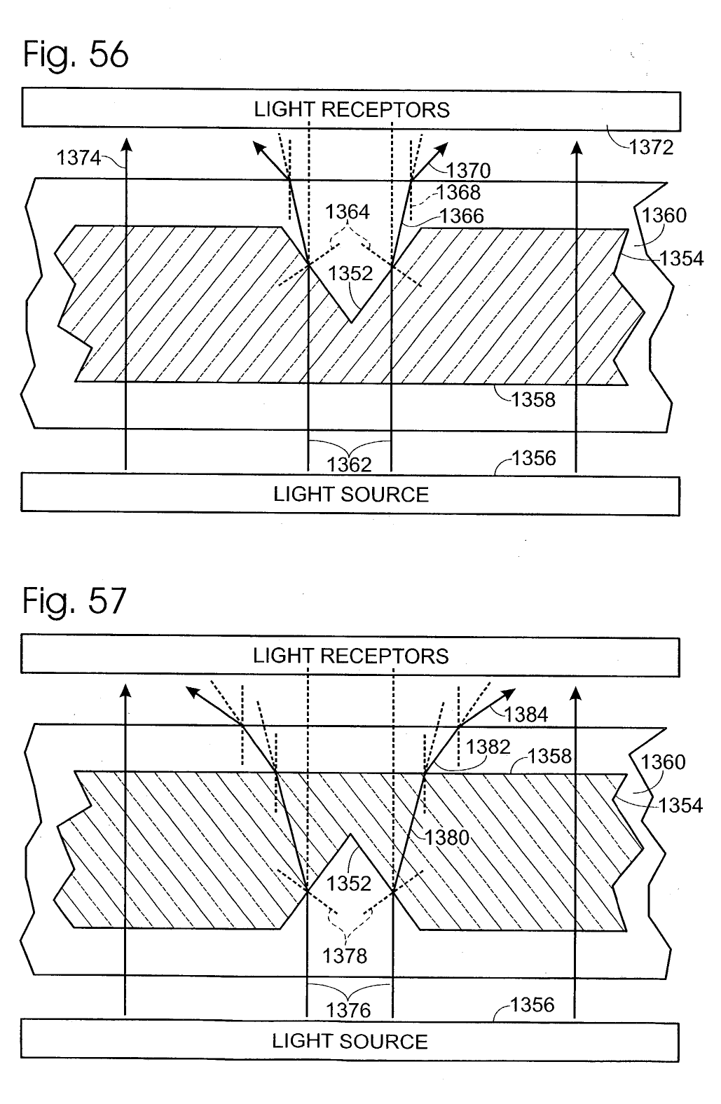

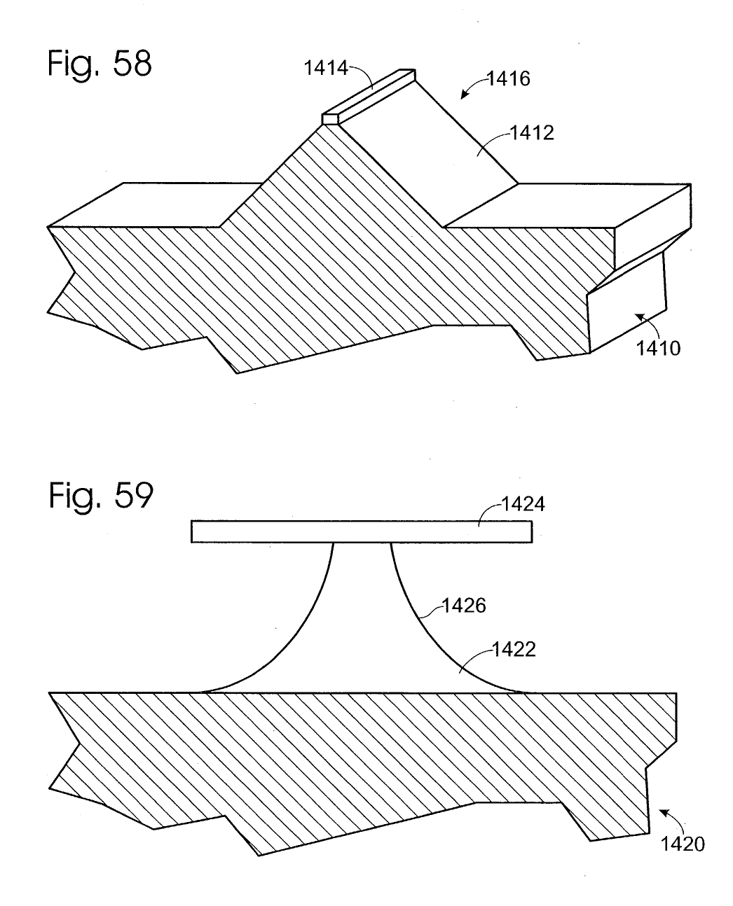

This section describes exemplary optical systems for reading topographic codes, particularly codes having recessed surface relief features; see FIGS. 56 and 57. Recessed features, such as surface relief features 1344 formed in sheet 1330, provide good optical contrast in transmitted light from particles, due to light refraction. As described below, the optical contrast is not dependent on the side of the particle facing the light source.

FIG. 56 shows light refraction produced by surface recesses facing away from a light source. Here, cone-shaped or pyramidal recess 1352 of particle 1354 faces away from light source 1356 and the non-indented side 1358 faces toward the light source. Particle 1354 is immersed in liquid 1360. The index of refraction (n) of the particle material (e.g., PMMA has n˜1.49) is higher than the index of refraction of liquid 1360 (e.g., water has n˜1.33), which in turn is higher than the index of reflection of air (n˜1.00). These differences in the respective indices of refraction cause light to diverge as it passes through the recess. Specifically, light 1362 is refracted away from normal, shown at 1364, as the light exits the particle, shown at 1366, and is further refracted away from normal 1368 as the light exits the liquid, shown at 1370. As a result, light receptors 1372 (photosensors, human eye, etc.) positioned above the particle sense a nonuniform pattern of light. Less light is transmitted from the recess, and so the recess looks dark, while surrounding areas look bright, because the light is not refracted (compare light beam 1374 with refracted beam 1370).

FIG. 57 shows light refraction produced by surface recesses facing toward from a light source. Here, cone-shaped or pyramidal recess 1352 of particle 1354 opposes light source 1356 and non-indented side 1358 faces away from the light source. As light beam 1376 meets recess 1352, the light is refracted toward normal 1378, shown at 1380. In contrast, light beam 1376 is refracted away from normal as it exits particle 1354 and liquid 1360, as shown at 1382 and 1384. Thus, light beams 1376 are divergently refracted, producing a nonuniform pattern of transmitted light, with a darkened region over recess 1352, as described above for FIG. 56.

5.7.4 Exemplary Die-Feature Geometries

This section describes exemplary die-feature geometries formed by anisotropic or isotropic etching; see FIGS. 58-59. Etching processes may be carried with monocrystalline silicon wafers, among others.

A. V-type Pyramid of 54° 44′ or 45°

FIG. 58 shows a V-type profile on a die 1410, which can be formed using silicon wafers with crystalline orientation (100) when the longitudinal direction of the ridges is <110>. The angle of side walls 1412 is 54°44′. This profile can be fabricated using a SiN mask 1414 on the pyramid top with the size of the mask and a pyramid 1416 defined by photolithography and any of the known etchants for anisotropic etching, such as KOH, NaOH, LiOH, EDP, hydrazine, gallic acid, TMAH, etc. Mask 1414 may be left on the die after etching because the undercut is usually insignificant.

An alternative V-type profile may be formed as a portion of a die on silicon wafers having a crystalline orientation of (100) or (110) when the longitudinal direction of the ridges is <100>. In this case, the angle of side walls 1412 is 45°. This profile can be fabricated as described above for the (100) crystalline orientation.

B. Funnel-type Protrusion

FIG. 59 shows a funnel-type profile on a die 1420, which can be formed on silicon wafers with any crystal orientation and any direction of the ridges. Protrusion 1422 may be manufactured by isotropic material removal, in both the non-masked open area of the wafer, as well as under a mask 1424. Mask 1424 may be designed to take into account etching that undercuts the mask. This profile can be fabricated with an isotropic wet etch, dry etch, or plasma etch, among others. The material of mask 1424 depends on the etching method, and may be SiN, SiO₂, metals like Al, Au, Ni, etc. The angle of side walls 1426 relative to the plane of the silicon wafer is variable, varying from close to 90° immediately adjacent mask 1424 to close to 0° near the at the base of protrusion 1422. Mask 1424 is generally stripped after etching.

5.8 Selected Embodiments II

This section describes further aspects of the invention, as set forth in the following numbered paragraphs:

1. A system for conducting a multiplexed experiment, comprising a set of microparticles including a first class of microparticles each having a detectably distinct first topographic code and a second class of microparticles each having a detectably distinct second topographic code, the first class of microparticles carrying a first sample, the second class of microparticles carrying a second sample, where the topographic code for each class of microparticles is at least partially formed by a surface structure of the microparticle, so that the set of microparticles can be analyzed in the same multiplexed experiment by identifying the first and second samples according to the topographic codes on their respective microparticles.

2. The system of paragraph 1, where each of the topographic codes includes at least one of a recess and a protrusion on a surface of the corresponding microparticle.

3. The system of paragraph 2, where at least one aspect of the at least one recess and protrusion forms the topographic code, the at least one aspect being selected from the group consisting of number, size, shape, position, height, and color.

4. The system of paragraph 1, where each topographic code is formed by plural recesses on the microparticle surface.

5. The system of paragraph 1, where the microparticles are at least generally planar.

6. The system of paragraph 1, where each topographic code is formed as an imprint in a particle precursor material.

7. The system of paragraph 6, where the particle precursor material is a sheet.

8. The system of paragraph 6, where each imprint including an imprint modifier, where the imprint modifier is added during formation of the imprint, and where the imprint modifier includes at least one of an optically contrasting material, a chemically reactive material, a magnetic material, and a sample for analysis.

9. A method of forming a topographic code on a microparticle, comprising (1) contacting a precursor material with a die under pressure, the precursor material being adapted to receive an imprint based on a surface structure of the die, and (2) partitioning the precursor material into plural microparticles, each of the plural microparticles including a detectable portion of the imprint, thus forming the topographic code.

10. The method of paragraph 9, where the precursor material is an at least generally planar sheet.

11. The method of paragraph 9, where the precursor material is a thermoplastic material.

12. The method of paragraph 9, where the precursor material is at least substantially colorless and transparent.

13. The method of paragraph 9, where partitioning is carried out after contacting.

14. The method of paragraph 9, where contacting forms guides, the guides being adapted to direct partitioning.

15. The method of paragraph 9, where the precursor material is heated to facilitate forming the imprint.

16. The method of paragraph 9, where the die includes monocrystalline silicon.

17. The method of paragraph 9, where the die includes surface relief features formed by anisotropic etching.

18. The method of paragraph 9, further comprising associating the die with imprint modifiers, where the die includes surface relief features, where the imprint modifiers are associated with the surface relief features, and the where imprint modifiers are at least partially transferred to the microparticles as a result of contacting.

19. A method of conducting a multiplexed experiment, comprising (1) arraying a set of microparticles including a first class of microparticles each having a detectably distinct first topographic code and a second class of microparticles each having a detectably distinct second topographic code, the first class of microparticles carrying a first sample, the second class of microparticles carrying a second sample, where the topographic code for each class of microparticles is at least partially formed by surface relief features of the microparticle, and (2) reading the first and second topographic codes to identify the first and second samples.

20. The method of paragraph 19, where reading includes identifying a region of optical nonuniformity on each microparticle.

21. The method of paragraph 18, where optical nonuniformity is identifiable by measuring light transmission from each microparticle.

22. The method of paragraph 18, where each topographic code is measured through divergent refraction of light.

### Example 6

### Particles Utilizing Molecular Imprinted Materials

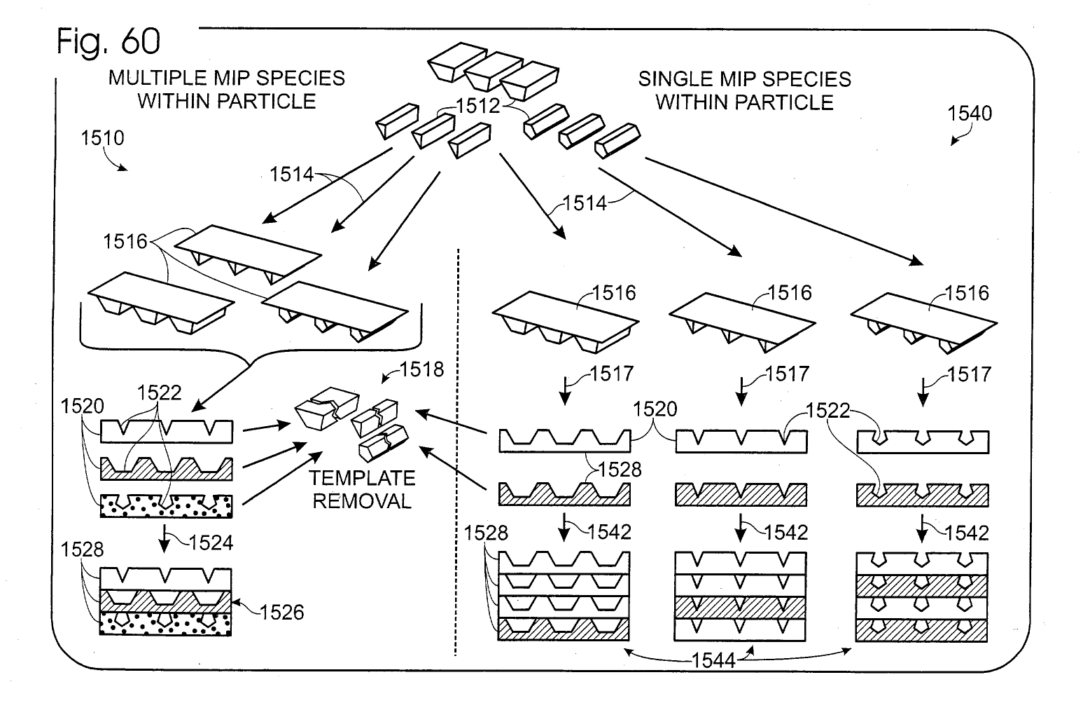

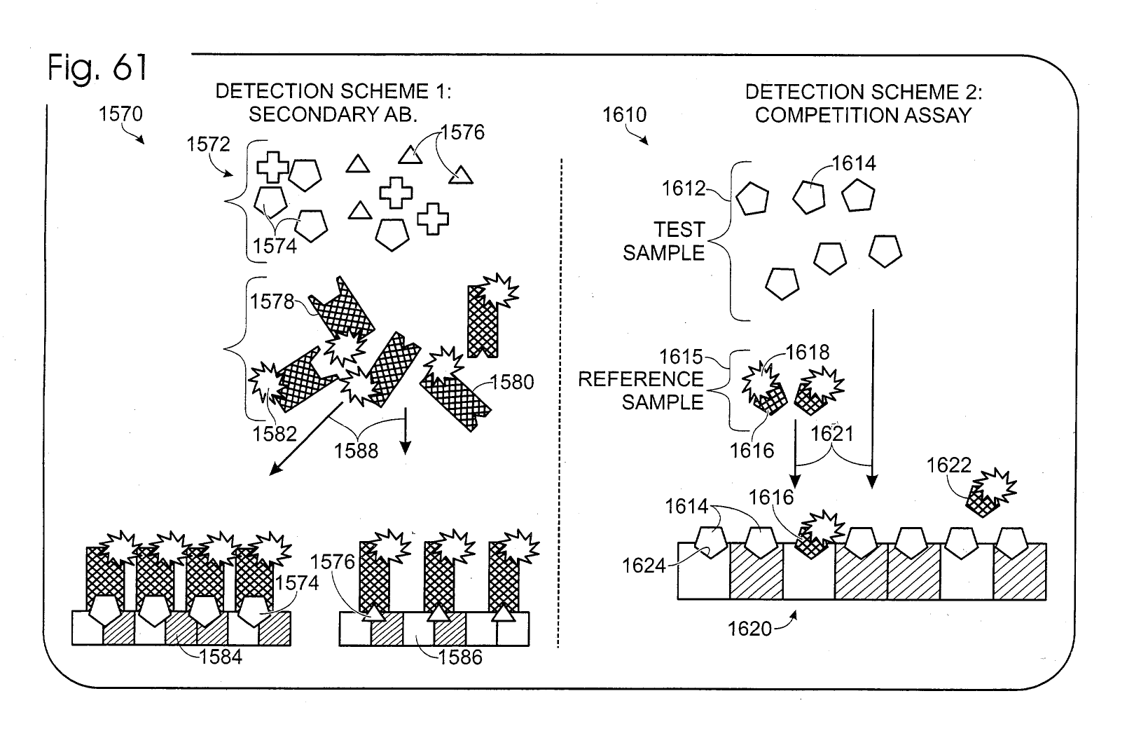

This example describes coded particles (or carriers) that include molecular imprinted materials; see FIGS. 60-61.

Antibodies are pivotal components of nature's most versatile and important surveillance system. Antibodies distinguish non-self from self, displaying a vast repertoire of potential binding specificities for molecules of virtually every shape, size, and functionality. The ability of the immune system to generate highly specific antibodies for a given antigen has promoted the widespread use of antibodies for analyte detection, measurement, and localization. Thus, antibodies play pivotal roles in virtually all aspects of biological analysis, acting as invaluable tools for clinical diagnosis and specific detection of biomolecules in biological systems.

Despite their value, antibody tools are not suitable for all applications. For example, some antigens do not generate a specific antibody response when exposed to a vertebrate immune system. These antigens may lack reactive epitopes due to their molecular structure or may not be recognized as foreign. Other antigens generate an immune response, but the resulting antibodies lack the necessary specificity. For example, antibodies reactive with a specific stereoisomer may also bind related, but distinct stereoisomers. A further problem, related to the use of animals, is the time and expense necessary to produce antibodies. Animals typically mount immune responses over the course of weeks or months, a time frame too slow for some research or clinical applications. In addition, immune responses are unpredictable, often varying between individual animals. As a result, several animals or more may be devoted to exposure with a single antigen, without a guarantee of success. Thus, the cost of animal housing may represent a substantial, sometimes prohibitive, barrier to antibody production.

Molecular imprinted polymers (MIPs) represent a possible alternative to antibodies. MIPs may function as synthetic antibodies produced as polymers molded around print molecules. The print molecules mold the forming polymer so that an imprint of the print molecule remains after the polymer has formed and the print molecules are removed. The resulting imprinted polymer then may be capable of binding the print molecule or a structurally related analyte with high affinity and specificity.

As synthetic antibodies, MIPs overcome many of the drawbacks of antibodies produced in vivo. For example, MIPs may be produced without animals, with greater speed and at lower cost. In addition, very specific MIPs may be generated against print molecules that cannot act as antigens in animal immune systems or that produce immune responses without the requisite specificity.

However, the generation and use of MIPs pose new challenges. Because MIPs may be produced rapidly, and at low cost, large numbers of MIPs may be generated for testing. In contrast to in vivo selection for antibodies with high affinity and specificity, optimal MIPs generally are produced by an empirical trial-and-error approach. Therefore, new systems are needed for identifying effective MIPs and for applying these effective MIPs to analysis of analytes in biological and environmental systems.

Systems are provided, including methods, compositions, apparatus, and kits for multiplexed analysis using coded particles that include molecular imprinted materials (MIMs). Each MIM may bind an analyte or set of analytes with high specificity and/or affinity. MIMs are formed as part of coded particles or are linked to coded particles after the particles are formed. As a result, each MIM is identifiable based on the code included on its particle, and, in some embodiments, based on MIM position within the particle. Coded particles with MIMs allow distinct MIMs to be combined and used together for multiplexed analysis of a common or distinct analytes. The invention thus may provide improved systems for analysis of, and with, MIMs, to improve MIM formulation and analyte measurement.

These improved systems may have a variety of advantages. For example, the improved systems may allow more rapid and less expensive identification of MIMs having the desired specificity and affinity. In addition, these systems may employ MIMs for multiplexed analysis of biological systems and/or test samples with greater efficiency and higher throughput than currently available approaches. Moreover, these systems may allow less expensive scale up, obviate the need for antibody binding chemistry, differentiate between molecules by structural and/or spatial differences, facilitate measurement of the levels and/or production of small molecules, and/or be stable under nonphysiological conditions (e.g., nonphysiological temperature, pH, etc.).

The following sections describe further aspects of the invention: (6.1) molecular imprinted material formation, including (1) print molecules, (2) polymer components, (3) polymerization, and (4) removal of print molecules; (6.2) MIM coded particles; (6.3) coded MIM arrays; (6.4) samples and sample binding to MIM coded particles; (6.5) measuring binding to MIM coded particles; (6.6) selected embodiments I; and (6.7) selected embodiments II.

6.1 Molecular Imprinted Material Formation

Molecular imprinted materials (MIMs) generally comprise any polymer or other material that is formed and/or solidified in the presence of one or more print molecules and then separated from at least some of the print molecules after polymer or material formation or solidification. Separation leaves an imprint of the print molecule in the polymer or other material. The polymer or material is formed outside of a biological system, such as a cell, a virus, or an animal. Polymers, termed molecular imprinted polymers (MIPs), are used typically to hold imprints, and thus will be used throughout as an exemplary MIM.

6.1.1 Print Molecules

Molecular imprinted materials are formed in the presence of print molecules. Print molecules generally comprise any atom, molecule, complex, or mixture used to form an imprint in a polymer, or other material, such as glass, ceramic, metal, or a composite, among others. A print molecule may be a pure substance, a combination of two or more pure substances, or a characterized or uncharacterized mixture of many substances. Exemplary print molecules include inorganic compounds, such as elements, ions, metals, acids, bases, salts, and metal complexes, among others. Other exemplary print molecules include simple monofunctional organic molecules, such as carboxylic acids, esters, ketones, ethers, amines, amides, thiols, and the like. Print molecules may also be polyfunctional organic molecules, such as amino acids, that have two or more similar or distinct functional groups. Additional exemplary print molecules may include oligomers and polymers, such as peptides, proteins, oligo- or polynucleotides, and/or carbohydrates, among others. Additional exemplary print molecules also may include biomolecules such as steroids, steroid hormones, lipids, phospholipids, prostaglandins, inositol triphosphate, diglycerides, amino acid derivatives, coenzymes, mononucleotides including adenosine, AMP, ADP, ATP, cAMP, cytosine, guanosine, and thymidine, vitamins, or other hormones, among others.

6.1.2 Polymer Components

Polymer components generally comprise any materials that are linked through molecular interactions to form linear and/or branched polymers. Molecular interactions may include covalent bonds, salt bridges, hydrogen bonds, electron sharing, and/or the like. Typically, polymer components are monomers. The monomers may be a single linkable species or a mixture of species with distinct structures. Monomers may include any suitable functional groups, or properties, such as hydrophobicity or hydrophilicity to interact with print molecules. Functional groups present on monomers may include any suitable moiety capable of contributing to monomer-print molecule interaction, polymerization, polymer physical properties, and/or polymer chemical properties. Exemplary functional groups may include carboxylic acids, esters, amines, amides, ketones, ethers, thiols, thioates, thioesters, phosphates, phosphonates, hydroxyls, and/or the like.

Prior to polymerization, polymer components typically are mixed with print molecules. As a result of mixing, the polymer components may complex with, bind to, or otherwise interact with print molecules by any suitable mechanism. Exemplary mechanisms include covalent bonds, hydrogen bonds, salt bridges, van der Waals interactions, electron sharing, and/or hydrophobic or hydrophilic attraction, among others.

6.1.3 Polymerization

Polymer components and print molecules are combined, and then the polymer components are polymerized. Polymerization generally comprises any mechanism that links the polymer components to form the polymers. Polymerization may be carried out by any suitable chemical reaction include polar reactions (nucleophilic or electrophilic attack), free radical addition, pericyclic reactions, and/or the like. In addition, polymerization may be catalyzed by any suitable chemical catalyst or promoter, including an acid, a base, a metal (such as a transition metal), a free radical initiator, and/or a solvent, among others. Polymerization also may be catalyzed by a physical catalyst or promoter, such as heat, light, pressure, and/or particle radiation, among others.

6.1.4 Removal of Print Molecules

After polymerization to form a MIM, print molecules are at least partially removed to leave unoccupied imprints. The removal of print molecules may be carried out by (1) altering the structure of the print molecule, for example, by chemical modification, (2) altering the strength and/or quality of the interaction between the print molecule and the polymer, and/or (3) increasing the probability of print molecule release. The structure of the print molecule may be altered by any suitable treatment that alters the structure, position; and/or modification of chemical bonds. Exemplary treatments may include hydrolysis, oxidation, modification, digestion, and/or the like. The strength and/or quality of the interaction between the print molecule and polymer may be altered by environmental changes, such as solvent properties, ionic strength, pH, temperature, and/or the like. The probability of print molecule release may be increased by mechanical or physical treatments. For example, the MIP may be pulverized to increase its surface-to-volume ratio. This may provide print molecules a greater chance to escape to surrounding solvent, if any.

6.2 MIM Coded Particles

The coded particles generally comprise populations of relatively small particles distinguishable at least in part by a detectable code. The particles may be formed substantially of MIMs or may include MIMs as a layer or coating, such as a surface film. In particular, if MIMs are pulverized to facilitate print molecule removal, the pulverized MIMs may be applied and adhered to the surface of the particle. In some embodiments, MIMs may be restricted to a compartment(s) of the particle.

The code may be a positional code with spatially distinct coding elements, as described elsewhere in this Detailed Description, particularly Section I. Each coding element may include a distinct MIM or MIMs, in addition to a distinct detectable property. Thus, MIM identity may be defined both by particle code and by position of the coding element within the particle.

6.3 Coded MIM Arrays

Coded MIM arrays may be formed from MIM coded particles. Coded MIM arrays generally comprise a set of coded MIM particles having distinct codes and/or distinct MIMs. A coded MIM array may be positional within a particle, as described above. In addition, a coded MIM array may include plural MIM coded particles with distinct codes that are nonpositionally and/or positionally arrayed. Nonpositional means that the position of each particle is not used to identify the linked MIM(s) and/or the sample exposed to the particle, and/or to interpret results. Nonpositional arrays of MIM coded particles may be formed by combining distinct coded particles. Such nonpositional arrays may be distributed to form sibling nonpositional arrays with substantially similar representation of particles. These sibling nonpositional arrays may be positionally distributed so that the position of each sibling array provides information about the particle, MIMs, sample, and/or analysis. Alternatively, or in addition, nonpositional arrays may be nonpositionally distributed, but identified based on an internal property of the array, such as a labeling particle, or an external property, such as a code or marking that identifies the entire array.

Exemplary coded arrays are described in more detail in the patent applications identified above under Cross-References and incorporated herein by reference, particularly U.S. patent application Ser. No. 09/694,077, filed Oct. 19, 2000; and U.S. patent application Ser. No. 10/120,900, filed Apr. 10, 2002.

6.4 Samples and Sample Binding to Coded MIM Arrays

Samples may be exposed to MIM coded particles to allow sample binding. Samples generally comprise any material that is being analyzed or used for analysis. Samples may be of known, partially known, or unknown composition, and may include solutions, mixtures, analytical test materials, and biological or environmental samples, among others. Samples of known composition may be suitable for comparing the binding specificity and/or affinity of different MIMs. Samples of partially known composition may be suitable for competition binding analysis, as described below.

Sample binding generally comprises measurable association between MIM coded particles and an analyte in the sample. Typically, sample binding represents molecular interaction between imprints in MIMs and the analyte. The analyte may be a major, minor, or trace component of a sample. In addition, the analyte may be structurally identical to a print molecule used to form the MIM, or may be structurally distinct. In some embodiments, the analyte that binds to a single MIM may represent a group of structurally related, but distinct molecules (or materials), or a structurally diverse set of molecules (or materials).

6.5 Measuring Binding to MIMs

After samples are exposed to MIM coded particles, analyte binding is measured, and the particle code may be read.

Binding of analyte to MIM coded particles may be measured directly, by quantifying bound analyte. Direct binding generally measures the presence, absence, or level of binding of the sample analyte by analyzing the analyte itself. Analyte may be directly measured using a labeled analyte. Analytes may be inherently labeled, that is, have a directly measurable property, or they may be labeled through modification or complex formation. Modification may include incorporating a detectable tag into the analyte. Exemplary tags include addition of radioisotopic tags, optically detectable tags, such as luminescent or fluorescent dyes, and/or detectable binding tags, such as biotin or digoxigenin. Alternatively, analytes may be detected through complex formation with a detectable binding agent. Binding agents include any material that specifically binds the analyte. Exemplary binding agents include specific antibodies that bind an analyte epitope that is not masked by binding to a MIM. Analyte binding to a MIM coded particle may be measured by directly detecting the amount of analyte bound to the particle.

Binding also may be measured indirectly. An exemplary indirect binding measurement uses a competition binding assay. In competitive binding, a known amount of a labeled analyte (or similarly competing material) is combined with an unknown amount of unlabeled analyte to be measured in a sample. The unlabeled analyte competes for binding sites on the MIM according to the concentration of unlabeled analyte.

Exemplary samples, assays, and methods for reading codes and measuring analyte binding for use with coded particles, such as MIM coded particles, are described in more detail elsewhere in this Detailed Description and in the patent applications identified above under Cross-References and incorporated herein by reference, particularly U.S. patent application Ser. No. 09/694,077, filed Oct. 19, 2000; and U.S. patent application Ser. No. 10/120,900, filed Apr. 10, 2002.

6.6 Selected Embodiments I

The following section describes selected aspects and embodiments of the invention, including methods for making and using coded MIM arrays, exemplified with MIPs. These aspects and embodiments are included for illustration and are not intended to limit or define the entire scope of the invention.

6.6.1 Methods of Producing MIP Particles

FIG. 60 illustrates embodiments of distinct methods for MIP particle production that create different MIP configurations within coded particles.

Method 1510 disposes a plurality of MIPs formed with distinct print molecules or “antigens” 1512 on a single coded particle. Print molecules 1512 are polymerized, for example by exposure to UV light, shown at 1514, to produce templated polymers 1516. Print molecules 1512 may be at least partially removed from polymers 1516, for example, by degradation, shown at 1517, to release template fragments 1518. Template release creates MIPs 1520 with unoccupied binding imprints 1522. Each MIP 1520 may be formed to provide an optically detectable code element, such as a color, by incorporating an optical agent, such as a dye. MIPs 1520 formed with distinct print molecules 1512 and thus different binding specificities may be joined, as shown at 1524. The resulting coded particle 1526 may have a plurality of binding specificities, each restricted to a distinct code element, 1528. The position of each code element within particle 1526 and/or an optical property of each code element may identify each MIP and MIP reactivity.

FIG. 60 also shows method 1540 that forms coded particles having only a single species of MIP. MIPs 1520 are formed similarly to method 1510. However, MIPs formed from the same template 1512 may define distinct code elements 1528 within a particle. Accordingly, joining distinct MIP layers, shown at 1542, forms coded particles with plural code element 1528, each defined by a similar MIP 1520. Alternatively, a coded particle may be produced first and then coated or layered with one or more MIPs (not shown).

In some embodiments MIP particles may be formed to bind to a large template or imprint molecule, such as an antibody. For example, antibody may be incorporated into a MIP by UV polymerization in the presence of polymer components. The resultant polymer may be joined with a coded particle or formed as part of a code. Following removal of print molecule antibodies, for example, by proteolysis, the coded particle may be used to detect antibodies that are structurally related to the template print molecules.

6.6.2 Detection Methods for MIP Particles

FIG. 61 illustrates different detection methods for measuring analytes in a multiplexed analysis with MIP particles. Method 1570 shows how MIP coded particles may be used to detect analytes in conjunction with conventional antibodies. A sample 1572 having analytes or antigens 1574, 1576 in a mixture a various species is combined with antibodies 1578, 1580, which each bind selectively to one of the analytes. The antibodies may include or interact with labels 1582, such as dyes, that make the antibodies detectable. The sample 1572 and antibodies 1578, 1580 are combined with MIP particles 1584, 1586, as shown at 1588. Each MIP particle 1584, 1586 is configured to specifically bind analyte 1574, 1576, respectively, and each analyte may correspond to template molecules used to form the MIPs. The antibodies are selected so that binding of analyte 1574, 1576 to each MIP particle does not preclude binding of antibody 1578, 1580 to the analyte. Accordingly, each analyte may act as a bridge that recruits label to each MIP particle. Therefore, the extent of particle labeling by antibody 1578, 1580 corresponds to the level of analyte 1574, 1576 in sample 1572, and reading the particle code identifies the analyte. In other embodiments, analytes and MIP particles may be combined before analyte is contacted with antibody.

Method 1610 shows how competitive binding may be used to detect analyte binding to MIP particles. Here, a test sample 1612 with an unknown amount of unlabeled (or labeled) test analyte 1614 is combined with a reference sample 1615 having a known amount of reference analyte 1616. Reference analyte 1616 may be similar or identical to test analyte 1614 but also may include a detectable label 1618 that distinguishes the reference and test analytes. Both test and reference analytes 1614, 1616 are combined with coded particle 1620, as shown at 1621. The amount of reference analyte 1616 bound to coded particle 1620 is an indirect measure of the amount of test analyte 1614 that competed for binding. For example, a reference analyte species, shown at 1622, has been displaced by competition from test analyte 1614 for a limited number of MIP-based template imprints 1624 on coded particle 1620. Accordingly, the signal measured from MIP particle 1620 is decreased correspondingly. Reading the particle code identifies the MIP attached to the particle and/or the analyte being measured.

6.7 Selected Embodiments II

This section describes selected embodiments of the invention, presented as a series of indexed paragraphs.

1. A particle with binding specificity for an analyte, comprising a particle, the particle including a code and a molecular imprinted material that specifically binds the analyte.

2. The particle of paragraph 1, where the molecular imprinted material was formed in the presence of a print molecule, the print molecule being selected from the group consisting of ligands, drugs, peptides, oligonucleotides, polynucleotides, toxins, analytes, ions, hormones, proteins, antigens, and antibodies.

3. The particle of paragraph 1, where the molecular imprinted material selectively binds a molecule selected from the group consisting of ligands, drugs, peptides, oligonucleotides, polynucleotides, toxins, analytes, ions, hormones, proteins, antigens, and antibodies.

4. The particle of paragraph 1, where the molecular imprinted material is at least substantially a polymer.

5. The particle of paragraph 4, where the polymer is formed from monomers, and the monomers include at least one functional group selected from the group consisting amines, carboxylic acids, hydroxyls, esters, ethers, and thiols.

6. The particle of paragraph 5, where the monomers include a mix of at least two different monomers.

7. The particle of paragraph 1, where the molecular imprinted material includes at least two different materials with at least substantially distinct analyte-binding specificities.

8. The particle of paragraph 7, where the at least two materials are spatially segregated.

9. The particle of paragraph 8, where the code includes positional code elements, and the at least two materials are disposed on different positional code elements.

10. The particle of paragraph 1, where the particle is formed at least substantially from the molecular imprinted material.

11. The particle of paragraph 1, where the particle includes an exterior surface, and the molecular imprinted material is disposed at least substantially at the exterior surface.

12. A method of forming a coded particle, comprising: imprinting plural molecular imprinted materials, each material including an optically detectable property; and linking the molecular imprinted materials in an array to form the particle, where the particle includes a code defined at least partially by the optically detectable property of each material in the array.

13. The method of paragraph 12, where the materials at least substantially include polymers.

14. The method of paragraph 12, where the plural materials are imprinted in the presence of at least substantially identical print molecules.

15. A method of detecting an analyte, comprising: combining the analyte and a particle, the particle including a code that identifies the analyte and a molecular imprinted material that selectively binds the analyte; and measuring an amount of the analyte bound to the particle.

16. The method paragraph 15, where the analyte is selected from the group consisting of a toxin, a drug, a ligand, a hormone, a receptor, an ion, a sugar, a lipid, a peptide, and an amino acid.

17. The method of paragraph 15, where the analyte is included in a sample, the sample being selected from the group consisting of blood, urine, perspiration, feces, sputum, mucus, lymph, serum, plasma, cell lysate, tissue lysate, water, and soil.

18. The method of paragraph 15, where measuring includes detecting the bound analyte with a specific binding agent that recognizes the analyte.

19. The method of paragraph 18, where the specific binding agent is an antibody.

20. The method of paragraph 15, where measuring includes detecting the bound analyte by competitive binding with a known amount of a substance, the substance binding specifically to the molecular imprinted material.

21. The method of paragraph 20, where the substance includes a label and a portion at least substantially identical to the analyte.

22. The method of paragraph 15, where the step of measuring further comprises: reading the code; and identifying the analyte based on the code.

23. A composition for multiplexed analysis of plural analytes, comprising: a first particle including a first code and a first molecular imprinted material, where the first code identifies a first analyte, and the first molecular imprinted material specifically binds the first analyte; and a second particle including a second code and a second molecular imprinted material, where the second code identifies a second analyte, and the second molecular imprinted material specifically binds the second analyte.

### Example 7

### Multiplexed Analysis Using Chromic Materials

The example describes coded particles (or carriers) having codes, particles, and/or biological samples/reagents that include, or are labeled with, chromic materials.

The analysis of biological systems often is facilitated by performing concurrent assays. For example, immunohistochemical analyses of cells often label two or more cell components at once, to provide internal controls and/or reference information, or simply to maximize efficiency of the analyses. Thus, sets of fluorescent dyes with nonoverlapping excitation and/or emission spectra have been developed to allow double-, triple-, quadruple-, and even higher-order labeling.

However, some concurrent analyses that include or produce colored materials suffer from significant optical interference. For example, cells stained for beta-galactosidase activity with a chromogenic substrate exhibit an intense color that interferes with other optical measurements of the cells. Similarly, when cells are grown on a colored substrate, such as a variably colored substrate that forms a code, analysis of the cells may be affected by a variable background signal from the substrate. Therefore, methods and systems are required for producing conditionally colored substrates, codes, and cells, to minimize optical interference during concurrent analyses.

Systems are provided for multiplexed analysis of biological systems using coded particles having codes, particles, samples, and/or sample probes that may include, or may be labeled with, chromic materials, i.e., materials that exhibit color changes in response to environmental changes. The, chromic materials may include photochromic materials that change color in response to light exposure. Chromic materials may form detectable aspects of the code and thus may be used to form a conditional code. Chromic materials also may be distributed generally within particles to influence global optical properties of coded particles. Furthermore, chromic materials may be used to label cells, thus providing a conditionally detectable sample characteristic. By conducting multiplexed assays with components that have conditional optical properties imparted by chromic materials, these assays may show reduced interference between the code and the sample during their analysis. The assays generally may be performed using particles, codes, samples, and assays described elsewhere in this Detailed Description and in the patent applications identified above under Cross-References and incorporated herein by reference.

The following sections describe further aspects of the invention, including (7.1) chromic materials, (7.2) codes with chromic materials, (7.3) particles with chromic materials, (7.4) labeling samples with chromic materials, (7.5) selected embodiments I, and (7.6) selected embodiments II.

7.1 Chromic Materials

Codes, particles, samples, and/or sample probes may include or be labeled with chromic materials. Chromic materials generally comprise any element, compound, polymer, complex, and/or mixture that exhibits an altered absorbance spectrum or intensity of light in response to an environmental change. The altered absorbance spectrum or intensity may be for visible light and may produce a change in the transmitted visible light that alters the color or the overall light transmittance of the chromic materials. For example, an environmental change may convert a chromic material from a generally colorless to a colored form (or vice versa), from one color to another color, such as from red to green, or from transparent to opaque. The change in light absorbance may be reversible or irreversible. When reversible, reversibility may be mediated by time and/or another environmental change.

Chromic materials may be activated or altered by an environmental change. An environmental change generally comprises exposure of the chromic materials to any altered physical or chemical condition that affects light absorption of the chromic materials. For example, chromic materials may be affected by light (photochromic materials), temperature (thermochromic materials), electric field (electrochtomic materials), pressure (batochromic materials), or pH (halochromic materials). Exemplary photochromic materials may be converted from a colorless to a colored form by exposure to UV light. In some cases, these exemplary materials may be converted back to a colorless form by exposure to visible light.

7.2 Codes with Chromic Materials

A particle code may be formed from chromic materials incorporated in and/or attached to a particle. The chromic materials may be introduced into the particle during particle formation. Alternatively, the chromic materials may be attached to the particle after particle formation, for example, as thin films or coatings. In either case, specific chromic materials may be present in spatially restricted portions of the particle to form a spatial code, such as a spatial color code. Furthermore, the code may not be detectable until revealed by the appropriate environmental change. Alternatively, the code may be detectable initially and then temp orarily or permanently lightened or removed with the appropriate environmental change.

In some embodiments, the code is a color code formed with distinct photochromic materials that transmit distinct sets of visible light wavelengths, and thus are colored distinctly. These photochromic materials may be colorless when illuminated with visible light in the absence of UV light. However, illumination with UV light may render the color code detectable with visible light, because the photochromic materials would be converted to their colored forms. A code formed from photochromic materials may be read by simultaneous and/or sequential illumination of the code with UV and visible light. For example, a particle may be epi-illuminated with UV light, while simultaneously measuring transmission of visible light through the particle and specific regions thereof.

A particle with a code formed from chromic materials, for example photochromic materials, may show minimal interference from the code when analyzing an associated sample. For example, the fluorescence intensity of the sample associated with the particle may be measured against a uniform background provided by a colorless substrate with a currently invisible code. In contrast, particles with codes formed by both colored and colorless bands show a fluorescence signal that is significantly higher over the colorless bands compared to that over the colored bands. As a result, total and local fluorescence emission from a particle is affected by its underlying code. However, with a code formed from chromic materials, the sample fluorescence may be measured when the code is colorless and invisible.

7.3 Particles with Chromic Materials

Photochromic materials also may be distributed more widely on or throughout a particle to improve optical properties of the particle. Widely distributed photochromic materials may be used to block optical signals from the particle itself and/or the particle code, either of which may interfere with measuring an optical property of the sample. For example, the particle and/or the particle code may produce background fluorescence that obscures a fluorescence signal from the sample. In this case, the widely distributed photochromic materials may be activated with UV light, thus darkening the photochromic materials and the particle, and blocking excitation of, and/or emission from, any fluorochromes in the particle. The darkened particle also may provide a uniformly dark background against which the sample signal may be read.

7.4 Labeling Samples using Chromic Materials

Photochromic materials also may be used as labeling reagents to reduce optical interference from the sample when reading the code. Such optical interference may occur when the sample is labeled with any strongly colored labeling reagent, such as a dye or a colored reaction product. By using a labeling reagent that is photochromic, the labeling reagent may be converted to a detectable form with light before and/or after, but generally not during, reading the code.

7.5 Selected Embodiments I

This section describes selected aspects and embodiments of the invention, namely use of a photochromic reaction product to measure beta-galactosidase activity in cells. The section illustrates how a light-activated labeling reagent may be produced and used, and is included for illustration without being intended to limit or define the entire scope of the invention.

In standard cellular assays, in situ beta-galactosidase activity is measured with a chromogenic substrate, such as X-Gal. Beta-galactosidase activity in cells converts X-Gal into an intensely colored blue compound that may obscure the particle code. However, problems with reading the particle code may be alleviated at least partially by creating a beta-galactosidase substrate that is converted to a photochromic reaction product by beta-galactosidase. Such a substrate may be developed by attaching a beta-galactoside to a photochromic material, preferably rendering the resulting hybrid material colorless and nonphotochromic. The attachment also may increase the solubility of the hybrid material relative to the unattached photochromic material in aqueous systems, as many photochromic materials are hydrophobic. When acted upon by beta-galactosidase in cells, beta-galactoside is cleaved from the hybrid material, thus releasing the insoluble photochromic reaction product, but in its unactivated, colorless state. The particle code then may be read without interference from the colorless reaction product. In addition, the reaction product may be converted to a colored form and thus measured by illuminating the cells with UV and visible light. Depending on the photochromic materials used, the light-activated reaction product may be black, a shade of gray, or one of many distinct colors.

7.6 Selected Embodiments II

This section describes selected embodiments of the invention, presented as a series of indexed paragraphs.

1. A system for multiplexed analysis of biological samples, comprising: a set of coded particles including a first particle with a first code and a second particle with a second code distinguishable from the first code, the coded particles being adapted to carry biological samples identified by the first and second codes, where the first particle includes a chromic material that becomes detectable or undetectable in response to an environmental treatment.

2. The system of paragraph 1, where the chromic material is distributed in a spatial pattern that is at least substantially independent of the first code.

3. The system of paragraph 1, where the spatial pattern is an at least substantially uniform distribution.

4. The system of paragraph 1, where the chromic material includes a photochromic property.

5. A system for multiplexed analysis of biological samples, comprising: a set of coded particles including a first particle with a first code and a second particle with a second code distinguishable from the first code, the coded particles being adapted to carry biological samples identified by the first and second codes, where the first and second codes are at least substantially undetectable with a first environmental condition, and detectable with a second environmental condition.

6. The system of paragraph 5, where the first and second codes are formed at least partially with chromic materials.

7. The system of paragraph 6, where the chromic materials include a photochromic property, and where the environmental condition is exposure to light.

8. The system of paragraph 7, where the light includes ultraviolet light.

9. The system of paragraph 5, where the environmental condition is at least substantially characterized by a parameter selected from the group consisting of pH, temperature, electric field strength, and pressure.

10. A system for multiplexed analysis of biological samples, comprising: a set of particle assemblies, each assembly including a particle, an optically detectable code, and a biological sample identified by the code, where each of the particle assemblies includes a chromic material that is detectable in response to an environmental treatment.

11. The system of paragraph 10, where the biological sample is labeled with a chromic material.

12. The system of paragraph 11, where labeling reveals an aspect of the biological sample selected from the group consisting of physical interaction, phenotypic interaction, and basic characteristic.

13. A method of analyzing biological samples, comprising the steps of: exposing a particle assembly to an analytical material, the particle assembly including a particle having a detectable code and supporting a biological sample identified by the code, where at least one of the particle, the code, the biological sample, and the analytical material includes a chromic material optically responsive to an environmental condition; treating the particle assembly with the environmental condition to create an altered optical property of the chromic material; and reading the code or measuring a characteristic of the biological sample while the optical property is altered.

14. The method of paragraph 13, where the particle includes the chromic material.

15. The method of paragraph 13, where the code includes the chromic material.

16. The method of paragraph 13, where the biological sample includes the chromic material.

17. The method of paragraph 13, where the analytical material includes the chromic material.

### Example 8

### Coded Particles with Metal Features

This example describes coded particles (or carriers) having metal features and also describes methods of forming the coded particles. The metal features may be formed on a transparent substrate, such as glass, and may be a single contiguous layer or plural contiguous or discrete layers. The metal features may confer distinct optical properties on a particle, either forming some or all of the particle's code and/or imparting measurable properties that facilitate analysis of an associated biological sample. Metal features may be restricted to a coding or noncoding portion of the coded particle, or may extend at least substantially over an entire surface(s) or volume of a particle. The invention may provide coded particles that are manufactured economically and have surfaces with strongly contrasting, well defined optical properties.

The following sections describe further aspects of the invention, including (8.1) metal features, (8.2) formation of metal features, (8.3) metal codes, (8.4) reading metal codes, (8.5) biological assays with coded particles having metal features, (8.6) selected embodiments I, and (8.7) selected embodiments II.

8.1 Metal Features

Coded particles are provided that have metal features on or in at least a portion of the particle. Metal features generally comprise any portion of one or more surfaces or compartments of the particle that includes metal atoms. Metal atoms generally include any metal from the periodic table, such as iron, gold, silver, copper, tin, aluminum, and so on. Metal features may include elemental metals, metal salts, metal oxides, metal alloys, sol-gels, organic metal oxides, organometallic materials, and/or combinations thereof, among others.

The metal features may have any suitable shape and size, based on the application. For example, metal features may be a thin film or films that extend over portions of a particle's surface. When a particle includes plural films, the films may be nonoverlapping, either contiguous or spaced; partially overlapping; and/or completely overlapping, for example, in. a stacked or layered relationship. The plural films may be generally coplanar, disposed on a common side or face of a particle. One or more films may be positionally disposed, so that each film has a defined or arbitrary position relative to the particle and/or each other. Furthermore, each film may have a similar or distinct thickness. Rather than simple films, metal features may extend into interior portions of the particle, and, in some cases, may extend to include substantial portions of the particle.

8.2 Formation of Metal Features

Metal features may be formed by any suitable method, based on the shape, size, composition, and number of features on each particle. For example, metal films may be formed by thin film deposition (e.g., metal vapor deposition), generally under vacuum conditions. Using this approach, plural distinct metal films may be formed sequentially or in parallel. Alternatively, or in addition, metal features may be formed using masks, such as photomasks, which define the position of each metal feature. Such masks may be combined with chemical etching to dispose metal features at positions on a surface of a particle. Furthermore, metal features may be formed by sol-gel processing. Suitable methods for disposing metal on a substrate are carried out commercially by Edmund Industrial Optics, Barrington, N.J., and Photo Sciences Inc., Torrance, Calif.

8.3 Metal Codes

Metal codes may be formed at least partially by metal features. The metal codes may be positional or nonpositional and may include one or more code elements. Each of the code elements may differ in a measurable property produced by composition, thickness, shape, size, and/or position, among others. For example, by varying the type of metal(s), its composition or alloying, and/or the thickness of the metal layer(s), different transmission, reflection, and/or absorption properties may be created.

Exemplary codes that may be formed partially or completely from metal features are described elsewhere in this Detailed Description, particularly in Section I and Example 2, and in the patent applications identified above under Cross-References and incorporated herein by reference, particularly U.S. patent application Ser. No. 09/694,077, filed Oct. 19, 2000; and U.S. patent application Ser. No. 10/120,900, filed Apr. 10, 2002.

8.4 Reading Metal Codes

Metal codes formed with metal features may be read using any detection system capable of detecting the metal features, including optical techniques, electrical techniques, and/or surface probe techniques, among others. Suitable techniques for reading metal codes are described elsewhere in this Detailed Description and in the patent applications identified above under Cross-References and incorporated herein by reference.

8.5 Biological Assays with Coded Particles Having Metal Features

The coded particles described herein generally may be used for studying any suitable sample using any suitable assays, such as those described elsewhere in this Detailed Description and in the patent applications identified above under Cross-References and incorporated herein by reference.

Coded particles with metal features also may be used in total internal reflection (TIR) assays. For example, a sample may be positioned on a metal feature and illuminated from the other side of the metal feature, at an angle of illumination greater than the critical angle. As a result, light is “totally” reflected internally, but portions of the sample that are very close to the surface feature are illuminated nevertheless. This approach may be useful to measure specific portions of a sample, such as a region of a cell very close to its surface membrane.

8.6 Selected Embodiments I

These sections describe selected aspects and embodiments of the invention, namely coded particles that include metal.

8.6.1 Particles with Metal Codes

This section describes coded particles having metal codes. Metal features may include metal codes formed on a common surface of a sheet of substrate material. Each code element may be a positionally defined metal film, or an absence of a metal film. For example, each code element may have one of plural potential optical properties, such as absorbance or reflectance of visible light, determined by the presence and/or absence, thickness, and/or composition of metal at a code element. The code elements may be restricted to a coding portion of each particle, leaving a noncoding portion for sample analysis, alternatively the code elements may be support or carry sample(s) and/or may participate in an assay.

The substrate may be a sheet of transparent material such as glass. The metal code elements may be disposed on a surface of the sheet, and then the sheet may be portioned into individual coded particles by cutting the sheet, for example, by mechanical, chemical, and/or optical means.

The composition of the substrate, and size, complexity, and optical properties of the code may be selected as appropriate for the assay. Thus, the substrate may be plastic, film, or composite, among others. Moreover, the code may be formed of larger or smaller numbers of code elements, and each of the code elements may have any number of possible optical properties. In addition, the metal code may occupy any suitable fraction of a surface of a coded particle.

8.6.2 Internal Metal Code Elements

This section describes a metal code formed at least partially with internal metal materials. A particle may be produced from layers of sol-gel materials, with at least some of the materials including metals. Alternatively, the layers may be made from thin films, such as plastics that include metals.

8.6.3 Metal Features in Sample Analysis

This section describes metal features that exploit the chemical and physical functionalities associated with various metals. The ability of distinct metals or metal compounds to catalyze various chemical reactions may be used to carry out a reaction(s) locally on a particle surface. The reactivity may facilitate sample analysis, binding, and/or detection, among others.

8.6.4 Hidden Metal Codes

This section describes covert or hidden metal codes. These codes may require further development to reveal the code. At least some of the metal code elements are coated with a compound or material that is not detectable without further reaction. The reaction may distinguish otherwise similar-appearing metal code elements.

8.7 Selected Embodiments II

This section describes selected embodiments of the invention, presented as a series of indexed paragraphs.

1. A particle for supporting biological samples, comprising: a substrate having an exterior surface; a feature disposed on the exterior surface, the feature including metal; and a code formed by at least one of the substrate and the feature.

2. The particle of paragraph 1, where the substrate is at least substantially planar.

3. The particle of paragraph 1, where the substrate at least substantially includes glass.

4. The particle of paragraph 1, where the exterior surface is adapted to be at least one of the upper and lower faces of the substrate during particle analysis.

5. The particle of paragraph 1, where the metal is from the group consisting of elemental metal, metal oxides, metal alloys, sol-gels, organic metal oxides, and organometallic materials.

6. The particle of paragraph 1, where the feature is formed by at least one film.

7. The particle of paragraph 1, where the feature at least substantially forms the code.

8. The particle of paragraph 1, where the feature includes plural spatially arrayed code elements.

9. The particle of paragraph 8, where each code element includes one of plural possible optical properties.

10. The particle of paragraph 9, where the plural optical properties are distinguishable by measuring a property from the group consisting of reflectance, absorbance, refraction, polarization, and luminescence.

11. A method of forming a coded particle, comprising: depositing a material on a surface of a sheet, the material including metal; and portioning the sheet and the material to form plural coded particles.

12. The method of paragraph 11, where the material is deposited as a film.

13. The method of paragraph 11, where the sheet at least substantially includes glass.

14. The method of paragraph 11, where the material is deposited at plural spaced regions of the sheet, the spaced regions being bounded at least substantially by future particle boundaries.

15. The method of paragraph 11, where the material is deposited to form plural code elements.

16. The method of paragraph 15, where each of the code elements has one of plural distinct thicknesses.

17. The method of paragraph 15, where the material includes plural distinct materials, and at least some of the distinct materials are deposited serially to distinct portions of the surface.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

We claim:

1. A set of devices for multiplexed analysis of samples or reagents, comprising:
plural particles, each particle having a code, the code being different for at least two of the particles, each particle including
an assay portion having a surface for association with at least one sample or reagent, the surface defining a perimeter having a pair of generally opposing edges, and
a frame portion defining the code and contrasting optically with the assay portion, the frame portion being disposed adjacent at least one of the opposing edges to flank the assay portion, thereby at least partially defining the perimeter.

2. The set of claim 1, where the frame portion has a defined position relative to the assay portion.

3. The set of claim 1, wherein the frame portion includes plural frame regions, the frame regions having a defined spacing relative to each other and a defined position relative to the assay portion.

4. The set of claim 1, the frame portion including at least one frame region that has a shape selected from the group consisting of spots, lines, bar, and circles.

5. The set of claim 1, wherein each particle has a perimeter at a position relative to the frame portion, positioning of the frame portion at least partially defining the position of the particle perimeter.

6. The set of claim 1, the assay portion being at least substantially colorless.

7. The set of claim 1, the surface being generally planar.

8. The set of claim 7, the surface including plural ridges.

9. The set of claim 1, wherein the frame portion includes a plurality of distinct frames regions that contrast optically with the assay portion, the plurality being disposed adjacent each of the opposing edges.

10. The set of claim 1, wherein only one of the frame regions defines the code.

11. The set of claim 1, the frame portion being colored.

12. The set of claim 1, further comprising a sample or reagent associated with each of the plural particles, at least two of the samples or reagents being different, the code identifying the associated sample or reagent.

13. A set of devices for multiplexed analysis of samples or reagents, comprising:
plural particles, each particle having a positional code, the code being different for at least two of the particles, each particle including
a coding portion defining the code and including plural discrete coding regions, and
a noncoding portion configured to be associated with at least one of the samples or reagents, the noncoding portion being disposed adjacent the coding portion and disposed between the plural coding regions so that such regions are spaced.

14. The set of claim 13, wherein the plural coding regions each have at least one color, the noncoding portion being at least substantially colorless.

15. The set of claim 13, wherein each of the particles is generally planar.

16. The set of claim 13, the coding and noncoding portions being defined by distinct structural components.

17. The set of claim 13, each particle being shaped generally as a parallelepiped.

18. The set of claim 13, wherein the noncoding portion includes a surface defining a perimeter with generally opposing edges, the coding regions defining colored bands that delineate at least two of the opposing edges.

19. The set of claim 13, wherein the noncoding portion is disposed at least substantially in a central region of each of the particles.

20. The set of claim 13, the noncoding portion and the coding regions being arrayed generally along a line, each particle appearing striped when viewed from a direction that is orthogonal to the line.

21. The set of claim 13, further comprising a sample or reagent associated with each particle, at least two of the samples or reagents being distinct, the code identifying the associated sample or reagent.

22. The set of claim 13, wherein the noncoding portion includes surface relief features.

23. The set of claim 22, the surface relief features including at least one groove.

24. The particle of claim 23, wherein each particle includes a pair of opposing edges, the at least one groove extending to each edge of the pair.

25. A set of devices for multiplexed analysis of samples or reagents, comprising:
plural particles, each particle having a code, the code being different for at least two of the particles, each particle including
a noncoding portion having a surface for association with at least one of the samples or reagents, the surface defining a perimeter having a pair of generally opposing edges, and
a coding portion defining a code and being disposed adjacent the noncoding portion near at least one of the opposing edges to at least partially frame the surface.

26. The set of claim 25, the plural particles being generally planar.

27. The set of claim 25, the code being positional.

28. The set of claim 25, wherein the coding portion includes plural coding regions disposed adjacent each of the opposing edges.

29. The set of claim 25, the noncoding portion being at least substantially colorless, the coding regions each including a color.

30. The set of claim 25, wherein the opposing edges are two generally parallel edges at which the noncoding portion is flanked by the coding region.

31. A set of devices for multiplexed analysis of samples or reagents, comprising:
plural particles, each particle having a code, the code being different for at least two of the particles, each particle including
a noncoding portion configured to be associated with at least one of the samples or reagents, the noncoding portion being at least substantially colorless and having a surface, the surface defining a perimeter having a pair of generally opposing edges, and
a colored portion defining a code, the colored portion being disposed adjacent the noncoding portion near at least one of the opposing edges so that the colored portion defines the perimeter near the at least one edge.

32. The set of claim 31, wherein the colored portion includes plural colored regions, the plural colored regions being disposed adjacent each of the opposing edges.

33. The set of claim 32, wherein only one of the colored regions defines the code.

34. The set of claim 31, the colored portion defining a positional code having plural code elements.

35. The set of claim 31, wherein the code is configured to be viewable from a direction generally normal to the surface.

36. A composition for multiplexed analysis, comprising:
a set of plural particles, each particle including plural discrete components attached to one another, at least two of the components being disposed in an array and cooperatively defining at least a portion of a positional code, the positional code being configured to be viewed from a direction generally normal to the array; and
a sample or reagent associated with each particle, at least two of the samples or reagents being different and associated with particles having different codes.

37. The composition of claim 36, the components being formed at least substantially of glass.

38. The composition of claim 36, the components being formed of polymeric materials or laminates.

39. The composition of claim 36, each of the at least two components having a position in the array and an optical property, the position and the optical property defining a code element of the positional code.

40. The composition of claim 39, wherein the optical property defines at least one color.

41. The composition of claim 40, wherein another of the components is at least substantially colorless.

42. The composition of claim 36, wherein one or more of the components is configured to be noncoding.

43. The composition of claim 42, wherein at least one of the noncoding components is disposed generally between the components that define the code.

44. The composition of claim 36, wherein one or more of the components has a surface that defines surface relief structure.

45. A set of devices for multiplexed analysis of cells, comprising: plural particles, each particle having
a surface defining at least one recessed region configured to at least partially receive one or more cells, and
a code that is optically detectable, the code being different for at least two particles of the set.

46. The set of claim 45, wherein each particle has a dcoding portion and a noncoding portion that are distinct, the coding portion defining the code, the noncoding portion at least partially defining the recessed region.

47. The set of claim 45, wherein the recessed region includes at least one groove.

48. The set of claim 47, wherein each particle has a surface, the surface including the recessed region and having an edge, the at least one groove being plural grooves that extend to the edge.

49. The set of claim 45, wherein each particle includes a nonrecessed region that borders the recessed region, the recessed region having a depth measured relative to the nonrecessed region, the depth being at least about equal to the average diameter of the one or more cells.

50. The set of claim 49, the nonrecessed region being at least one ridge that extends generally parallel to the recessed region.

51. The set of claim 45, further comprising plural cell populations that provide the one or more cells, each of the cell populations being associated with one of the particles so that the code identifies the associated cell population and the one or more cells are at least partially received in the recessed region.

52. The set of claim 45, the code being a positional color code.

53. The set of claim 45, wherein the recessed region is a groove that extends generally along a line, the code including plural code elements arrayed generally orthogonal to the line.

54. A set of devices for analysis of cells, comprising:
plural particles, each of the particles having an exterior surface and a code that is optically detectable, the exterior surface including plural grooves configured to at least partially receive one or more of the cells, the code of at least two of the particles being distinct.

55. A method of making coded particles for analysis of a sample, comprising:
providing a progenitor structure having a surface with plural surface regions;
shaping the surface to create relief structure on each of the plural surface regions;
cutting the progenitor structure into plural particles so that each of the plural surface regions is disposed on a different one of the plural particles; and
forming a code on each of the plural particles.

56. The method of claim 55, wherein the progenitor structure includes plural discrete structural components disposed in a bundle having a diameter, the method further comprising the steps of attaching the structural components to each other and stretching the structural components to decrease the diameter.

57. The method of claim 55, wherein the step of shaping includes forming grooves on the surface, the grooves having a length that is shortened by the step of cutting.

58. The method of claim 55, wherein the step of shaping includes chemically etching a selected portion of each particle.

59. The method of claim 55, wherein the code is arrayed generally along a line or a plane, the step of cutting forming a cut that is at least substantially parallel to the line or plane.

60. The method of claim 55, wherein the progenitor structure is formed of plural discrete structural components.

61. The method of claim 56, wherein at least one of the structural components provides the surface, the step of shaping being performed on the at least one structural component before the bundle is formed.

62. A method of analyzing samples, comprising:
distributing a set of particles on a surface of a substrate, each particle having a generally planar surface bounded by an edge, the generally planar surface defining at least one recessed region;
associating the set of particles with plural samples, at least two of the particles having a different code and being associated with a distinct one of the samples, each distinct sample being at least partially disposed in a compartment defined between the recessed region and the substrate surface, the compartment having an open end; and
exposing the set of particles to a reagent so that the reagent contacts the sample by entering the open end of the compartment.

63. The method of claim 62, the recessed region including at least one groove.

64. The method of claim 62, the compartment being elongate and having opposing ends, each of the opposing ends being open.

65. The method of claim 62, wherein the step of associating is performed before the step of distributing.
